(19) United States
(12) Patent Application Publication
(10) Pub. No.: US 2025/0101412 A1
(43) Pub. Date: Mar. 27, 2025

(54) METHODS AND SYSTEMS FOR INCREASING CAPTURE POTENTIAL OF A SPATIAL ARRAY

(71) Applicant: OREGON HEALTH & SCIENCE UNIVERSITY, Portland, OR (US)

(72) Inventors: Yu-Jui Chiu, Portland, OR (US); Fehmi Civitci, Portland, OR (US); Eugene Tu, Portland, OR (US); Andy Mendoza, Portland, OR (US); Nicholas Groves, Portland, OR (US)

(21) Appl. No.: 18/832,900

(22) PCT Filed: Jan. 25, 2023
(86) PCT No.: PCT/US2023/061258

§ 371 Date: Jul. 24, 2024

Related U.S. Application Data
(60) Provisional application No. 63/302,706, filed on Jan. 25, 2022.

(51) Int. Cl. C12N 15/10

(52) U.S. Cl. CPC C12N 15/1065

(57) ABSTRACT

An IC spatial array for a captured ligand assay is described. Materials to prepare and methods to produce the IC spatial array are also provided. The IC spatial array includes a matrix substrate including capture probes anchored to the matrix substrate such that the capture probes have a barcode unique to the capture probe's spatial location within the matrix substrate and the capture probe has a free end for capturing analytes from a sample.

### CROSS-REFERENCE TO RELATED APPLICATIONS

This application claims priority to U.S. Provisional Patent Application No. 63/302,706, filed Jan. 25, 2022, which is incorporated herein by reference in its entirety as if fully set forth herein.

### REFERENCE TO SEQUENCE LISTING

The Sequence Listing associated with this application is provided in XML format in lieu of a paper copy and is hereby incorporated by reference into the specification. The name of the XML file containing the Sequence Listing is 0046-0057PCT.xml. The file is 14.1 KB, was created on Jan. 24, 2023, and is being submitted electronically via Patent Center.

### FIELD OF THE DISCLOSURE

The current disclosure describes a spatial array with increased capture potential in a captured ligand assay. The present disclosure also provides materials to prepare and methods to produce the increased capture potential spatial array for a captured ligand assay.

### BACKGROUND OF THE DISCLOSURE

Understanding the spatial organization and molecular characteristics of cells within a tissue is important in the study of normal development and disease pathology. Spatial transcriptomics is a molecular profiling method that allows for the measurement of gene activity within a sample while mapping where that activity is occurring.

By placing a sample on a slide with an array of discrete areas, each discrete area containing spatially-barcoded capture probes, cDNA libraries are generated with precise positional information. In current spatial transcriptomic platforms, an array of specialized capture probes are attached to the surface of glass slides. These capture probes are designed to capture molecules from a sample such as, in many scenarios, RNA. RNA-capture probes include a spatial barcode unique to a location on the array and an element to capture the RNA such as a polyT sequence. When a sample, such as a tissue, is placed on the slide, the RNA from the sample is able to bind to the adjacent RNA-capture probe. A reverse transcription reaction generates cDNA of the captured mRNA while incorporating the spatial barcode. Thus, a cDNA library is generated linking any RNA sequencing data to the location on the array and subsequently the location on the sample. This provides for the analysis and characterization of transcriptomes and/or genomic variations while preserving spatial information.

Although this technique enables complex bioinformatic analysis by merging imaging, sequencing, and molecular characterization, it is limited by the amount of RNA-capture probes that can be attached to the surface of the slide. When molecules are attached to a two-dimensional surface, the quantity of molecules that can be attached is limited by the area of the surface and the repulsive intermolecular forces between the molecules being attached. The quantity of RNA that is able to be collected from a sample is therefore limited by the number of RNA-capture probes. If the array has a low capture potential, or less capture probes per area, some signals may be missed. For example, mRNAs expressed by the sample at lower copy number may not be captured and thus will not be detected. Thus, there is a need for a spatial array with improved capture potential and a method to perform spatial transcriptomics with better discovery potential.

### SUMMARY OF THE DISCLOSURE

The present disclosure describes an improved capture potential (IC) spatial array. The present disclosure also provides materials to prepare and methods to produce the IC spatial array. The IC spatial array is produced by projecting a template array into a three dimensional matrix substrate such that the resulting matrix substrate contains an array of discrete volumes, each discrete volume including spatially-barcoded capture probes, along a plane of the matrix substrate and continuing into its thickness. Because of its thickness, the IC spatial array has increased capture potential because of the increased number of capture probes per area, as compared to a conventional template array.

The present disclosure provides starting materials for preparing the IC spatial array. In particular embodiments, the starting materials include a starting matrix substrate and a template array (FIG. 1B). In particular embodiments, the starting matrix substrate includes primer docking elements (PDEs) and capture sequence primers (CSPs (e.g., a polyA sequence in FIG. 1B)), wherein the PDEs are anchored to the matrix substrate. The CSPs can be anchored to the matrix substrate or not anchored to the matrix substrate. In particular embodiments, the template array includes a substrate including an array with a plurality of discrete areas, each discrete area includes template probes immobilized to the substrate such that each template probe has a free end and each template probe includes: a PDE template (PDET), a barcode template including a nucleotide sequence unique to a particular discrete area, and a capture sequence template (CST (e.g., a polyT sequence in FIG. 1B).

A starting matrix substrate is placed on a template array in PCR-enabling media, wherein the starting matrix substrate includes PDEs and CSPs, wherein the PDEs are nucleotide sequences anchored to the matrix substrate and the template array includes a substrate including an array with a plurality of discrete areas, wherein the discrete areas form a 2-dimensional spatially-defined map (e.g., grid) and each discrete area includes a plurality of template probes immobilized to the substrate such that each template probe has a free end and each template probe is a nucleotide sequence including (from the 5′ to 3′ end): a PDET, a barcode template including a nucleotide sequence unique to the discrete area, and a CST. In particular embodiments, the CSP hybridizes to (e.g., a complementary nucleotide sequence to) the CST. In particular embodiments, the PCR-enabling media includes PCR master mix and DNA polymerase. During a PCR cycle, temperature is increased and decreased to amplify, or copy a provided nucleotide sequence. When the mixture is cooled, the CSP migrates and anneals with the CST (FIG. 1C). At this point, the DNA polymerase synthesizes new strands of DNA starting at the annealed CST, producing a complementary sequence of (or sequence that hybridizes to) the template probe to create what is herein referred to as a bridge probe (FIG. 1D). In particular embodiments, the bridge probe includes a bridge PDE, a complement to the barcode referred to herein as a bridge barcode (or complementary barcode), and the CSP. In particular embodiments, the bridge PDE hybridizes to (e.g., is sufficiently complementary to) the PDE anchored to the matrix substrate and therefore hybridizes with the anchored PDE (FIG. 1E), resulting in the bridge probe linked to the matrix substrate (FIG. 1F). During another PCR cycle, the anchored PDEs are extended through the bridge probe to create a capture probe (FIG. 1G) and the bridge probe will dehybridize from the capture probe (FIG. 1H). In particular embodiments, the capture probe hybridizes (e.g., is sufficiently complementary) to the bridge probe and identical to the template probe. In particular embodiments, the capture probe includes a PDE, a barcode, and a capture sequence. In subsequent PCR cycles, the bridge probes hybridize with remaining anchored PDEs within the matrix substrate and extend through the bridge probe to create additional capture probes. This results in an IC spatial array including a matrix substrate having an array and a thickness, wherein the array includes a plurality of discrete volumes, each discrete volume occupying a distinct x and y position on the array and continuing into the thickness of the matrix substrate in the z direction, each discrete volume including a plurality of capture probes anchored to the matrix substrate such that the capture probes have a free end and each capture probe and include a barcode including a nucleotide sequence unique to the discrete volume in which the capture probe is anchored; and a capture sequence.

In particular embodiments, the capture probe can be synthesized using ligation instead of polymerization. For example, to synthesize a capture probe using ligation, a template strand and capture strand segments that hybridize to (e.g., are complementary to) the template strand are provided (FIG. 8). In particular embodiments, the template strand includes a PDET, a sequence encoding a sequence that hybridizes to (e.g., complementary sequence) the barcode and a CST. In particular embodiments, the sequence encoding the sequence that hybridizes to the barcode is divided into at least two template strand segments. Each template strand segment hybridizes to a capture strand segment. In particular embodiments, the capture strand segments include: a first half of the barcode and 6 or more nucleotide bases flanking each side of the first half of the barcode, and a second half of the barcode and 6 or more nucleotide bases flanking each side of the second half of the barcode. In particular embodiments, the template strand and capture strand segments are mixed with hybridization buffer and allowed to anneal. During annealing, the first half of the barcode and second half of the barcode will hybridize to the template strand. After this, the DNA ligase, DNA ligase buffer, and a CSP is added to the mixture. In particular embodiments, the DNA ligase catalyzes phosphodiester bonds at single-strand breaks in DNA between the 3′OH group and 5′-monophophate to form a capture probe that hybridizes to the template sequence. DNA ligation results in formation of a capture probe. After ligation, the capture probe can be dissociated from the template strand. In particular embodiments, the capture probe hybridizes to the template strand. In particular embodiments, the capture probe includes a PDE, capture strand segments, and a capture sequence primer (CSP).

The IC spatial array can be used to analyze the location and quantity of analytes within a sample using spatial transcriptomics. The IC spatial array improves the capture potential of the array by increasing the number of capture probes within the discrete volumes. In particular embodiments, the discrete volumes within the matrix substrate are not separated by a physical structure. In particular embodiments, the discrete volumes within the matrix substrate are separated by a physical structure such as in a capillary array. If the sample is transferred to the IC spatial array using electrophoresis, lateral diffusion of the analytes during transfer is reduced, thus improving the accuracy of analyte location information. The IC spatial array can also have an improved resolution by either isometrically expanding the sample or projecting capture probes into an expanded matrix substrate before shrinking the matrix substrate during production of the IC spatial array.

### BRIEF DESCRIPTION OF THE FIGURES

Some of the drawings submitted herewith may be better understood in color. Applicant considers the color versions of the drawings as part of the original submission and reserves the right to present color images of the drawings in later proceedings.

FIGS. 1A-1H. (1A) Overview of the mechanism and design of transferring 2D spatial barcodes to a matrix substrate (e.g., hydrogel layer) which is further described in FIGS. 1B-1F, depicting the projection of a template probe into a matrix substrate. (1B) A template probe is attached to a substrate. The template probe includes a primer docking element template (PDET), a barcode template, and a capture sequence template (CST) (e.g., T tail or polyT sequence). A matrix substrate includes a capture sequence primer (CSP) (e.g., A tail or polyA sequence) and a PDE anchored to the matrix substrate. (1C) When the matrix substrate is put in contact with the substrate and its associated template probe, the complementary capture domains (capture sequence, CSP, and CST) associate with each other. (1D) Running a polymerase chain reaction (PCR) cycle extends from the A tail to construct a bridge probe that is sufficiently complementary to the template probe. (1E) This bridge probe includes a bridge PDE that is sufficiently complementary to the PDE anchored within the matrix substrate, so the bridge PDE hybridizes with the matrix substrate-anchored PDE. (1F) With the bridge probe associated with the anchored PDE, (1G) another PCR cycle extends through the bridge probe to create a capture probe. (1H) The bridge probe dehybridizes from the capture probe and the capture probe is anchored to the matrix substrate through the anchored PDE.

FIG. 2. Adjacent capture probe sequences (capture probes within the same feature (e.g., discrete area or discrete volume)) use the same barcodes, PDEs, and capture sequences (e.g., T[20] tails). Because of this characteristic, all of the capture probes will be substantially equally amplified and transferred during PCR cycles. The competition between adjacent capture probes will ensure that resolution is maintained in the capture probe transfer step.

FIG. 3. TBE-Urea gel results from DNA elution at 95° C. after capture probe transfer into a matrix substrate. A very tight 80 bases band confirms that the correct PCR product was amplified during the described processes.

FIG. 4. Confocal results show successful capture probe transfer to a matrix substrate hydrogel layer.

FIG. 5. Additional proof of capture probe transfer into a matrix substrate. The yellow rectangle area in the drawing illustrates a bleaching area with higher magnification and light intensity which causes a dark area (on the right panel) when imaging a larger field-of-view area. Only fixed fluorophores cause this bleaching effect.

FIGS. 6A, 6B. Setup for electrophoretic enhanced RNA capture. (6A) The tissue section is placed on the matrix substrate (e.g., hydrogel) containing capture probes. The tissue section and the hydrogel are sandwiched by two metal plates and two buffer reservoirs. The buffer can be liquid or a soaked polymeric layer containing a high ion concentration for electrophoresis. If a 10-100V voltage bias is applied to the metal plates for 10-120 seconds, mRNA will migrate from the tissue section into the matrix substrate. (6B). A depletion layer can capture the majority of house-keeping genes, and allow the barcoded matrix substrate hydrogel layer to capture more rare RNA.

FIG. 7. Example of analytes (or modified analyte) and its associated capture probe. The example analytes show the characteristics of the target RNA, target DNA, and target protein that allow it to bind to the capture probe. The characteristics of the analyte can be inherent or can be engineered to include the characteristic. For example, a capture probe including a polyT capture sequence will capture target RNA or target DNA having a polyA tail. A target protein can also be captured by the polyT tail if the target protein is modified to include a polyA tail. One method of modifying the target protein to include a polyA tail is to target the target protein with an antibody that has a polyA tail.

FIG. 8. Besides using polymerase to build the second strand, a second strand was built by ligation. The barcode has been divided to two parts, each part has half of the barcodes with at least a 6 nucleotide flanking sequence on both sides. Building second strand by ligation can bypass oligo defects during DNA synthesis which potentially blocks enzymatic activity of polymerases.

FIG. 9. The barcode can be transferred to capillary arrays which were pre-functionalized with oligos or pre-filled with hydrogel. The capillary arrays provide physical confinement of the spatial barcodes, a capillary array can act as the mother plate, and it can be used for multiple barcode transfer. The capillary arrays can also act as the capture substrates.

FIG. 10. RNA library analysis by Agilent Bioanalyzer system. The results show similarity of RNA from tissue section and total RNA which demonstrate high quality RNA during electrophoresis and library preparation process.

FIG. 11. Immunofluorescent images after RNA capture shows tissue is intact after electrophoresis process. The quality of the tissue is valid for other proteomic analysis.

FIG. 12. Example of discrete volumes within a matrix substrate. Discrete volumes occupy discrete x and y locations in a matrix substrate and extend into the thickness of the matrix substrate. Capture probes within a discrete volume have a barcode that is unique to the discrete volume.

### DETAILED DESCRIPTION

Understanding the spatial organization and molecular characteristics of cells within a tissue is important in the study of normal development and disease pathology. Spatial transcriptomics is a molecular profiling method that allows for the measurement of gene activity within a sample while mapping where that activity is occurring.

By placing a sample on a slide with an array of discrete areas, each discrete area containing spatially-barcoded capture probes, cDNA libraries are generated with precise positional information. In current spatial transcriptomic platforms, an array of specialized capture probes are attached to the surface of glass slides. These capture probes are designed to capture molecules from a sample such as, in many scenarios, RNA. RNA-capture probes include a spatial barcode indicative of its location on the array and an element to capture the RNA such as a polyT sequence. When a sample, such as a tissue, is placed on the slide, the RNA from the sample is able to bind to the adjacent RNA-capture probe. A reverse transcription reaction generates cDNA of the captured mRNA while incorporating the spatial barcode. Thus, a cDNA library is generated linking any RNA sequencing data to the location on the array and subsequently the location on the sample. This provides for the analysis and characterization of transcriptomes and/or genomic variations while preserving spatial information.

Although this technique enables complex bioinformatic analysis by merging imaging, sequencing, and molecular characterization, it is limited by the amount of RNA-capture probes that can be attached to the surface of the slide. When molecules are attached to a two-dimensional surface, the quantity of molecules that can be attached is limited by the area of the surface and the repulsive intermolecular forces between the molecules being attached. The quantity of RNA that is able to be collected from a sample is therefore limited by the number of RNA-capture probes. If the array has a low capture potential, or less capture probes per area, some signals may be missed. For example, mRNAs expressed by the sample at lower copy number may not be captured and thus will not be detected. Thus, there is a need for a spatial array with improved capture and a method to perform spatial transcriptomics with better discovery potential.

Projecting a traditional template array into a three dimensional matrix substrate overcomes the constraints of limited area for molecules to bind by allowing molecules to be distributed throughout a volume. The IC spatial array can be used to analyze the location and quantity of analytes within a sample using spatial transcriptomics.

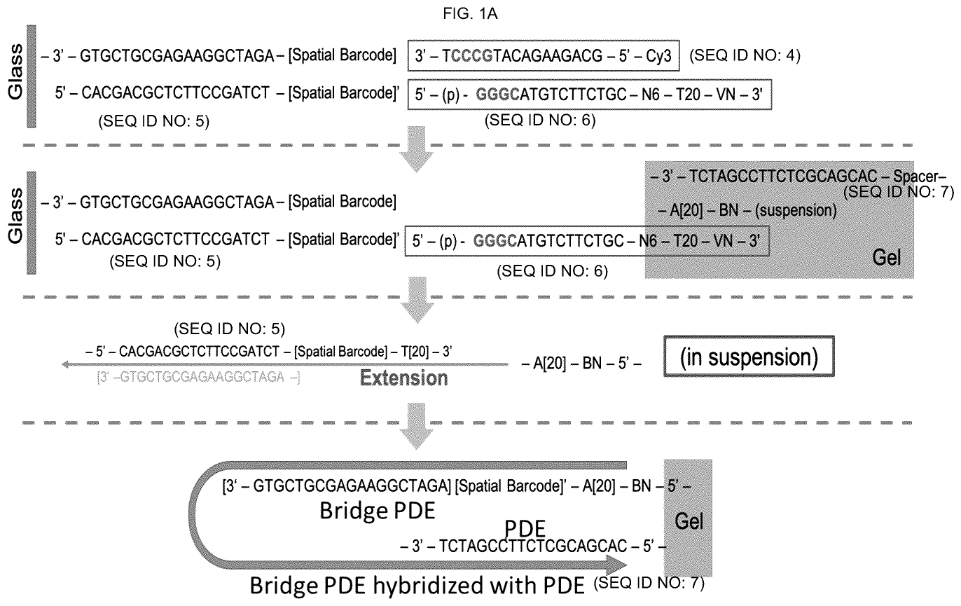

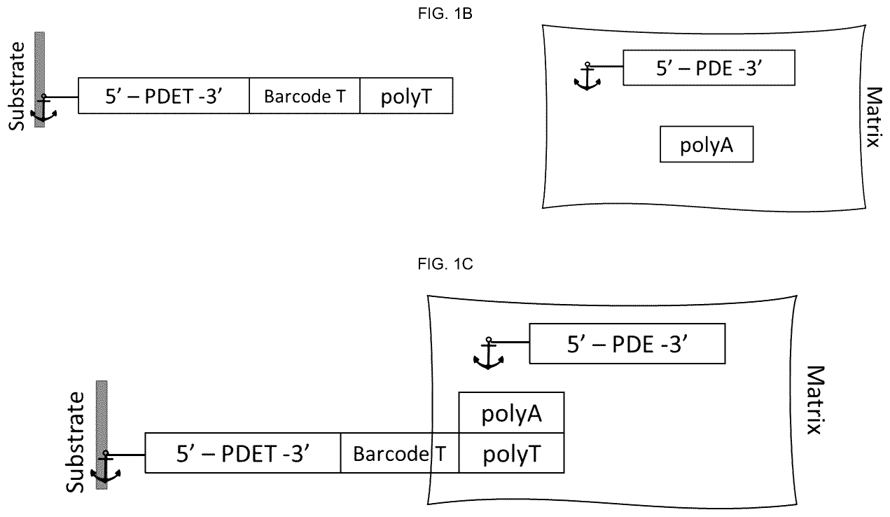

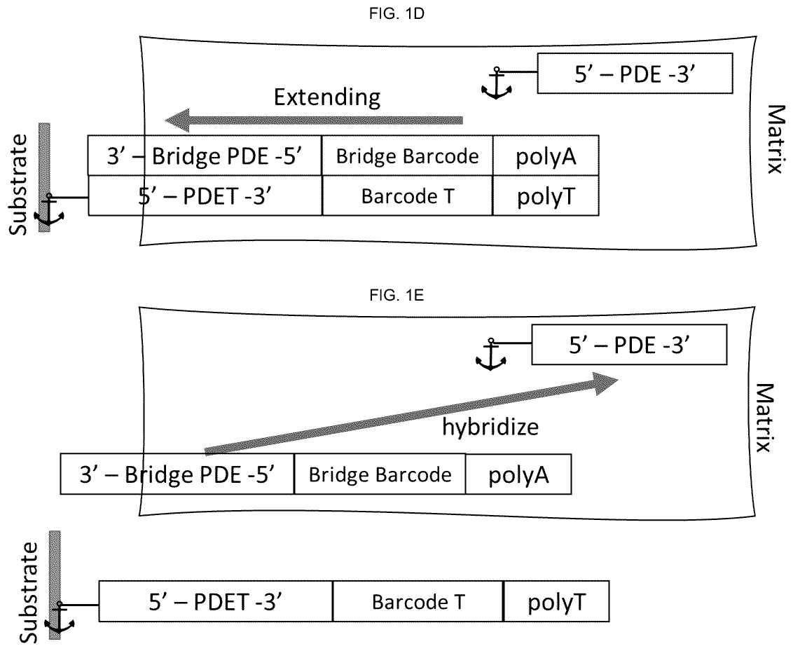

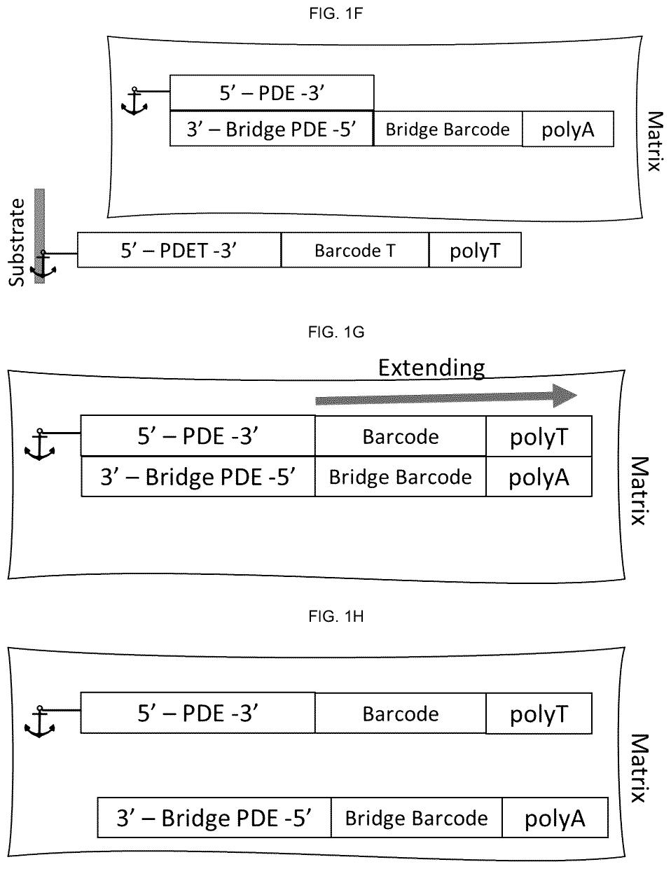

The present disclosure provides starting materials for preparing the IC spatial array. In particular embodiments, the starting materials include a starting matrix substrate and a template array (FIG. 1B). In particular embodiments, the starting matrix substrate includes primer docking elements (PDEs) (also referred to as final PDEs) and capture sequence primers (CSPs), wherein the PDEs are anchored to the matrix substrate. The CSPs can be anchored to the matrix substrate or not anchored to the matrix substrate. In particular embodiments, the template array includes a substrate including an array with a plurality of discrete areas, each discrete area occupying a discrete location on the array and including template probes immobilized to the substrate such that each template probe has a free end and each template probe includes: a PDE template (PDET), a barcode template including a nucleotide sequence identifying the discrete area, and a capture sequence template (CST).

In particular embodiments, the starting matrix substrate is made of an appropriate hydrogel that does not significantly degrade under reaction conditions including PCR or electrophoresis. In particular embodiments, the CST on the template probe is a reverse complement of the CSP. In particular embodiments, the CST readily anneals with the CSP within the matrix substrate. In particular embodiments, the CSP within the matrix substrate includes a poly A sequence and the CST includes a poly T sequence. In particular embodiments, the CSP within the matrix substrate includes a poly T sequence and the CST includes a poly A sequence (see, e.g., FIG. 1C). In particular embodiments, the starting matrix substrate does not separate discrete volumes with a physical structure. In particular embodiments, the starting matrix substrate separates discrete volumes by a physical structure such as in a capillary array.

To prepare the IC spatial array, a starting matrix substrate is placed on a template array in PCR-enabling media, wherein the starting matrix substrate includes PDEs and CSPs, wherein the PDEs are nucleotide sequences anchored to the matrix substrate and the template array includes a substrate including an array with a plurality of discrete areas, wherein the discrete areas form a 2-dimensional spatially-defined map (e.g., grid) and each discrete areas includes a plurality of template probes immobilized to the substrate such that each template probe has a free end and each template probe is a nucleotide sequence including (from the 5′ to 3′ end): a PDET, a barcode template including a nucleotide sequence unique to the discrete area, and a CST. In particular embodiments, the CSP hybridizes to the CST. In particular embodiments, the PCR-enabling media includes PCR master mix and DNA polymerase. During a PCR cycle, temperature is increased and decreased to amplify, or copy a provided nucleotide sequence. When the mixture is cooled, the CSP migrates and anneals with the CST (FIG. 1C). At this point, the DNA polymerase synthesizes new strands of DNA starting at the annealed CST, producing a sequence that hybridizes to the template probe to create what is herein referred to as a bridge probe (FIG. 1D). In particular embodiments, the bridge probe includes a bridge PDE, a complement to the barcode referred to herein as a bridge barcode (or complementary barcode or barcode’), and the CSP. In particular embodiments, the bridge PDE hybridizes to the PDE anchored to the matrix substrate and therefore hybridizes with the anchored PDE (FIG. 1E), resulting in the bridge probe linked to the matrix substrate (FIG. 1F). During another PCR cycle, the anchored PDEs are extended through the bridge probe to create a capture probe (FIG. 1G) and the bridge probe will be dehybridize from the capture probe (FIG. 1H). In particular embodiments, the capture probe hybridizes to the bridge probe and identical to the template probe. In particular embodiments, the capture probe includes a PDE, a barcode, and a capture sequence. In subsequent PCR cycles, the bridge probes hybridize with remaining anchored PDEs within the matrix substrate and extend through the bridge probe to create additional capture probes. This results in an IC spatial array including a matrix substrate having an array and a thickness, wherein the array includes a plurality of discrete volumes, each discrete volume occupying a distinct x and y position on the array and continuing into the thickness of the matrix substrate in the z direction, each discrete volume including a plurality of capture probes anchored to the matrix substrate such that the capture probes have a free end and each capture probe includes a barcode including a nucleotide sequence unique to the discrete volume in which the capture probe is anchored; and a capture sequence.

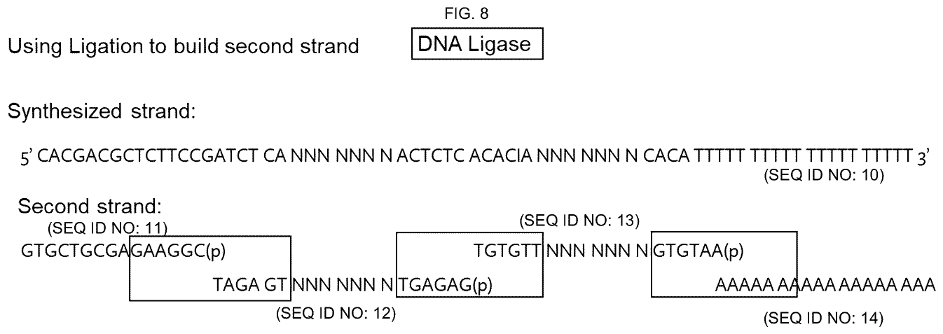

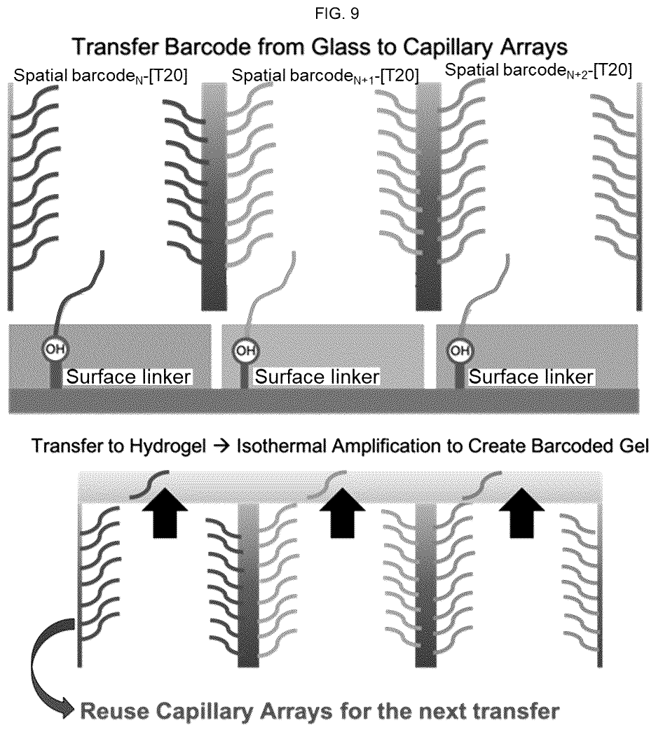

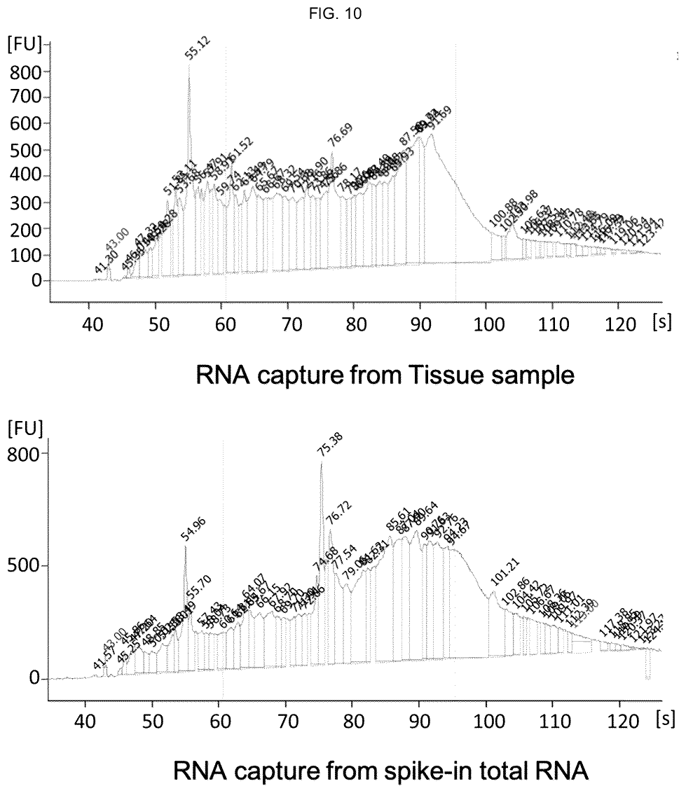

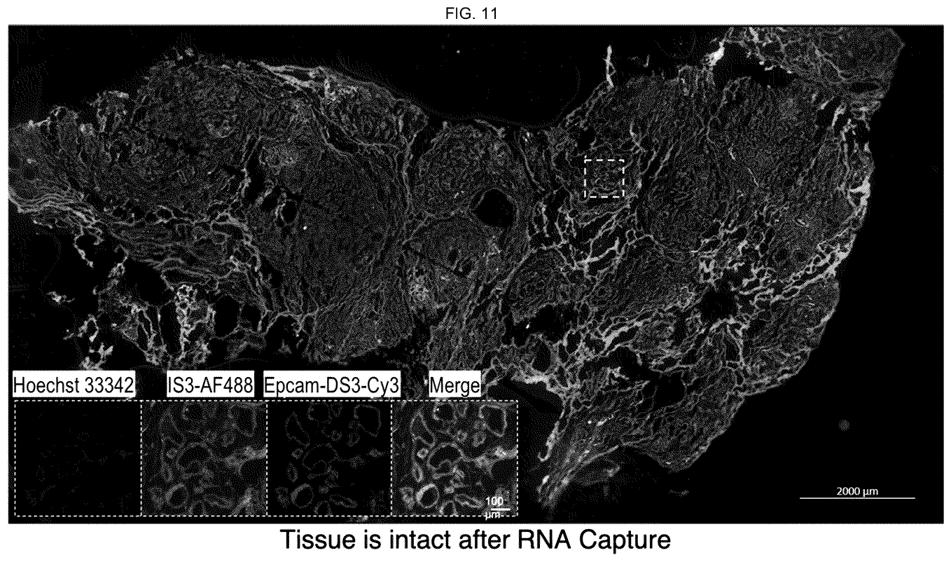

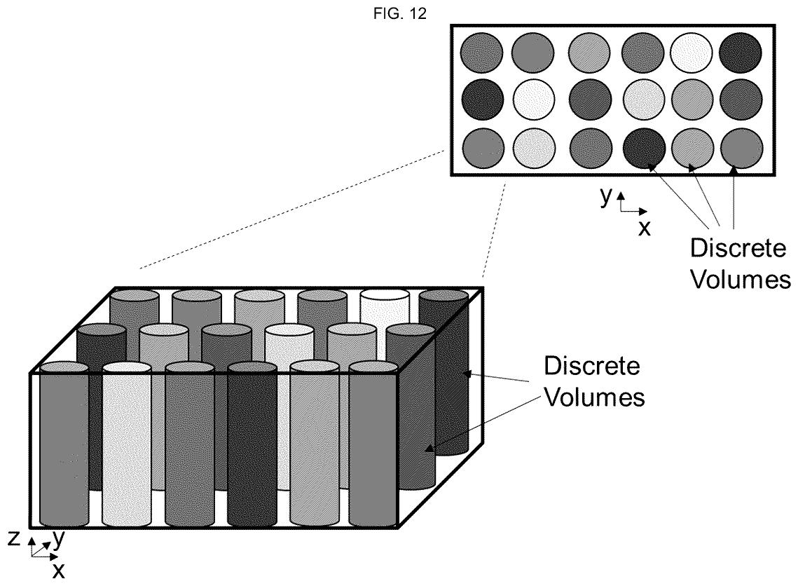

In particular embodiments, the capture probe can be synthesized using ligation instead of polymerization. For example, to synthesize a capture probe using ligation, a template strand and capture strand segments that hybridize to the template strand are provided (FIG. 8). In particular embodiments, the template strand includes a PDET, a sequence encoding a complementary sequence to the barcode and a CST. In particular embodiments, the sequence encoding the complementary sequence to the barcode is divided into at least two template strand segments. Each template strand segment hybridizes to a capture strand segment. In particular embodiments, the capture strand segments include: a first half of the barcode and 6 or more nucleotide bases flanking each side of the first half of the barcode, and a second half of the barcode and 6 or more nucleotide bases flanking each side of the second half of the barcode. In particular embodiments, the template strand and capture strand segments are mixed with hybridization buffer and allowed to anneal. During annealing, the first half of the barcode and second half of the barcode will hybridize to the template strand. After this, the DNA ligase, DNA ligase buffer, and a CSP is added to the mixture. In particular embodiments, the DNA ligase catalyzes phosphodiester bonds at single-strand breaks in DNA between the 3′OH group and 5′-monophophate to form a capture probe that hybridizes to the template sequence. DNA ligation results in formation of a capture probe. After ligation, the capture probe can be dissociated from the template strand. In particular embodiments, the capture probe hybridizes to the template strand. In particular embodiments, the capture probe includes a PDE, capture strand segments, and a capture sequence primer (CSP).

In particular embodiments, a feature includes a discrete area and/or discrete volume. In particular embodiments, a discrete area is a feature within a template array. In particular embodiments, a discrete volume is a feature within a three dimensional array (e.g., IC spatial array). In particular embodiments, the discrete volumes within the matrix substrate are not separated by a physical structure. In particular embodiments, the discrete volumes within the matrix substrate are separated by a physical structure such as in a capillary array. In particular embodiments, the capillary array can be used as a starting matrix substrate, a template array, or a matrix substrate.

In particular embodiments, the PCR-enabling media includes PCR master mix and Taq DNA Polymerase.

In particular embodiments, the capture probes anchored to the matrix substrate are designed to capture genomic, transcriptomic, or proteomic material from a sample. In particular embodiments, the matrix substrate is made of an appropriate hydrogel that does not significantly degrade under reaction conditions including PCR or electrophoresis. In particular embodiments, the capture sequence includes a poly A sequence or a poly T sequence. In particular embodiments, the capture sequence includes a recognition sequence.

The IC spatial array described herein increases the capture potential over traditional spatial arrays. Although the density of capture probes within a plane of the IC spatial array is similar to a traditional spatial array, the volume of the IC spatial array allows that density to be projected throughout the thickness of the array. This allows the discrete volume size to stay small, while capturing more analytes (molecule to be captured). Maintaining discrete volume size maintains the resolution of the spatial array.

Additional embodiments allow for enhanced versatility of the IC spatial array. The capture sequences of the IC spatial array can be designed to capture RNA, DNA, or proteins. Additionally, the molecules intended to be captured by the capture probes (analytes) can be modified to better interact with the capture probes.

The IC spatial array can be used to analyze the location and quantity of analytes within a sample using spatial transcriptomics. If the sample is transferred to the IC spatial array using electrophoresis, lateral diffusion of the analytes during transfer is reduced, thus improving the accuracy of analyte location information. The IC spatial array can also have an improved resolution by either isometrically expanding the sample or projecting capture probes into an expanded matrix substrate before shrinking the matrix substrate during production of the IC spatial array.

Aspects of the current disclosure are now described in more supporting detail as follows: (i) Probes; (i-a) Primer Docking Element (PDE) Domains; (i-b) Spatial Barcodes; (i-c) Capture Domains; (i-d) Other Functional Sequences; (ii) Template Array; (iii) Array Features; (iv) Matrix Substrate; (v) Projection into Increased Capture Spatial Array; (vi) Isometric Expansion; (vii) Shrinking the Array; (viii) Method of Use; (viii-a) Types of Samples; (viii-b) Sample Preparation; (viii-b-1) Sectioning; (viii-b-2) Freezing; (viii-b-3) Fixation and Embedding; (viii-b-4) Staining; (viii-b-5) Permeabilization; (viii-b-6) Analyte Enrichment; (viii-b-7) Clearing Step; (viii-b-8) Other Preparation Steps; (viii-c) Sample Transfer and Analyte Capture; (viii-d) Depletion Layer; (viii-e) Analyte Product Analysis; (viii-f) Spatial Analysis Methods; (ix) Exemplary Embodiments; (x) Example; and (xi) Closing Paragraphs. These headings are provided for organizational purposes only and do not limit the scope or interpretation of the disclosure.
(i) Probes. A probe, including a capture probe, a template probe, or a bridge probe, refers to any molecule (e.g., an oligonucleotide) that is capable of capturing analyte of interest from a sample. In particular embodiments, the capture probe is a nucleic acid molecule. In particular embodiments, a capture probe is in an IC spatial array. In particular embodiments, a template probe is on a template array and is used to project a capture probe into an IC spatial array. In particular embodiments, the capture probe hybridizes to the bridge probe. In particular embodiments, the capture probe is sufficiently complementary to the bridge probe. In particular embodiments, the bridge probe hybridizes to the template probe. In particular embodiments, the bridge probe is sufficiently complementary to the template probe. In particular embodiments, the capture probe is identical to the template probe. In particular embodiments, a probe includes a PDE domain, a spatial barcode, and a capture domain. In particular embodiments, the capture probe includes a PDE, a barcode, and a capture sequence. In particular embodiments, a bridge probe includes a bridge PDE, a bridge barcode, and a CSP. In particular embodiments, a template probe includes a PDET, a barcode template, and a CST. The probe can also include other functional sequences that are useful for subsequent processing. Functional sequences can include a cleavage domain, unique molecular identifier, or a spacer. The capture probe and the template probe have the same components and sequences (e.g., PDE/PDET, barcode/barcode template, and capture sequence/CST). The capture probe and the bridge probe have similar components, however the components in the bridge probe are reverse complements to the components of the capture probe.

An oligonucleotide is a linear polymer of nucleotides. Oligonucleotides are single stranded. Oligonucleotides can be of various lengths. Oligonucleotides can include modified nucleotides as known in the art.
(i-a) Primer Docking Element (PDE) Domains.

In particular embodiments, a probe includes a PDE domain. In particular embodiments, a capture probe includes a PDE. In particular embodiments, a bridge probe includes a bridge PDE. In particular embodiments, a template probe includes a PDET. In particular embodiments, a PDE hybridizes (e.g., is sufficiently complementary to) to a bridge PDE. A PDE domain, including a PDE, a PDET, and/or a bridge PDE, is a single stranded nucleotide sequence. Herein, the PDE functions as a matrix substrate-anchored sequence that hybridizes (e.g., is sufficiently complementary to) to the bridge PDE, allowing for the docking of the bridge probe to the anchored PDE such that a PCR cycle can extend through the bridge probe to create a capture probe.

In this disclosure, a matrix substrate includes a PDE sequence linked to the matrix substrate and a template array includes template probes including the same PDE sequence (PDET), a spatial barcode (barcode template), and capture domain (CST). When this template probe is amplified using PCR, a hybridizing sequence referred to as a bridge probe is produced. Because this sequence includes a bridge PDE, or PDE complementary to PDET and complementary to the PDE anchored to the matrix substrate, it will hybridize with the PDE linked to the matrix substrate.

Hybridize refers to a nucleotide sequence of a single-stranded nucleic acid molecule forming a complex with a nucleic acid molecule (e.g., having a sufficiently complementary nucleotide sequence). Generally, the complex forms through hydrogen bonding between complementary nucleotide bases in separate nucleic acid molecules.
(i-b) Spatial Barcodes.

A spatial barcode includes a barcode on the capture probe, a bridge barcode, and/or a template barcode. In particular embodiments, probes (capture probes, bridge probes, and template probes) each include at least one spatial barcode. In particular embodiments, probes each include 1, 2, 3, 4, 5, 6, 7, 8, 9, or 10 spatial barcodes.

In particular embodiments, a capture probe includes at least one spatial barcode (barcode). In particular embodiments, a bridge probe includes at least one spatial barcode (bridge barcode). In particular embodiments, a template probe includes at least one spatial barcode (barcode template). In particular embodiments, the bridge barcode hybridizes to the barcode and the barcode template.

A spatial barcode refers to a contiguous nucleic acid segment or two or more non-contiguous nucleic acid segments that function as a label or identifier that conveys or can convey spatial information. In particular embodiments, the spatial barcodes are associated with locations within the template array, IC spatial array, or sample.

A spatial barcode can function both as a spatial barcode and as a unique molecular identifier (UMI), associated with a probe or capture domain. Spatial barcodes can have a variety of different formats. For example, spatial barcodes can include polynucleotide spatial barcodes, random nucleotide sequences, and synthetic nucleotide sequences. In particular embodiments, the spatial barcode can allow for identification and/or quantification of individual sequencing-reads. In particular embodiments, the spatial barcode can be a fluorescent spatial barcode for which fluorescently labeled oligonucleotide probes hybridize to the spatial barcode. In particular embodiments, the spatial barcode can be a nucleotide sequence that does not substantially hybridize to mRNA molecules in a biological tissue. In particular embodiments, the spatial barcode has less than 80% sequence identity (e.g., less than 70%, 60%, 50%, or less than 40% sequence identity) to the nucleotide sequences across a substantial part (e.g., 80% or more) of the mRNA molecules in the biological sample. The spatial barcode sequences can include from 5 to 20 or more nucleotides within the sequence of the capture probe, bridge probe, or template probe. For example, the length of a spatial barcode sequence can be 5, 6, 7, 8, 9, 10, 11, 12, 13, 14, 15, 16, 17, 18, 19, 20 nucleotides or longer. In particular embodiments, the length of a spatial barcode sequence can be at least 5, 6, 7, 8, 9, 10, 11, 12, 13, 14, 15, 16, 17, 18, 19, 20 nucleotides or longer, or at most 5, 6, 7, 8, 9, 10, 11, 12, 13, 14, 15, 16, 17, 18, 19, 20 nucleotides or shorter.

A spatial barcode can include contiguous nucleotides, e.g., in a single stretch of adjacent nucleotides, or they can be separated into two or more subsequences that are separated by 1 or more nucleotides. Separated spatial barcode subsequences can be from 4 to 16 nucleotides in length. In particular embodiments, the spatial barcode subsequence can be 4, 5, 6, 7, 8, 9, 10, 11, 12, 13, 14, 15, 16 nucleotides or longer, or at least 4, 5, 6, 7, 8, 9, 10, 11, 12, 13, 14, 15, 16 nucleotides or longer, at most 4, 5, 6, 7, 8, 9, 10, 11, 12, 13, 14, 15, 16 nucleotides or shorter.

In particular embodiments, an IC spatial array includes a matrix substrate including an array and a thickness, wherein the matrix substrate includes a plurality of discrete volumes, each discrete volume occupying a distinct x and y position on the array and continuing into the thickness of the matrix substrate in the z direction, each discrete volume including a plurality of capture probes anchored to the matrix substrate.

In particular embodiments, the discrete volumes (or features) in the IC spatial array are derived from the discrete areas (or features) in the template array. The discrete areas on the template array occupy a distinct x and y position on the array such that the discrete area occupies a discrete two dimensional area on the template array.

In particular embodiments, multiple probes are attached to a common array discrete volume. In particular embodiments, each discrete volume includes multiple probes. In particular embodiments, the multiple probes that are attached to a common array discrete volume include one or more spatial barcode sequences that are unique to the discrete volume. In other words, all of the probes coupled to a single discrete volume have the same spatial barcode which differs from the spatial barcode on probes coupled to different discrete volumes. In particular embodiments, the multiple probes that are attached to a single discrete volume can include spatial barcodes that are different across all probes coupled to the discrete volume. In particular embodiments, probes from different array discrete volumes include different spatial barcodes such that each array discrete volume has a unique spatial barcode.
(i-c) Capture Domains. Herein, a capture domain refers to capture sequences, CSTs, and/or CSPs. Each capture domain can be a functional nucleic acid sequence configured to interact with a target sequence. The target sequence can be inherent to an analyte such as the polyA tail of mRNA or the target sequence can be added to an analyte.

The term capture refers to the capability of a first substance to interact with and/or bind a second substance where, for example, the second substance is part of a population of other substances. In particular embodiments, an analyte may be captured. An analyte refers to molecules, substances, structures, moieties, or components from or produced by a sample to be analyzed. Chemically, cellular analytes may include proteins, polypeptides, peptides, saccharides, polysaccharides, lipids, DNA, RNA, other nucleic acids, and other biomolecules. In a captured ligand assay, an analyte is the substance whose chemical constituents are being identified and/or measured.

A capture domain refers to a part of a molecule that is capable of binding or capturing a substance. A capture domain may be capable of capturing analytes that may include DNA, RNA, other nucleotides, proteins, polypeptides, peptides, saccharides, polysaccharides, lipids, and other biomolecules. In particular embodiments, an analyte includes RNA, DNA, proteins, or peptides.

Capture domains can include ribonucleotides and/or deoxyribonucleotides as well as synthetic nucleotide residues that can participate in Watson-Crick type or analogous base pair interactions. The capture domains can prime a reverse transcription reaction to generate cDNA that hybridizes to the captured mRNA molecules. The capture domains can prime a reverse transcription reaction to generate cDNA that is sufficiently complementary to the captured mRNA molecules.

Base paired refers to the situation where two complementary nucleic acids have formed hydrogen bonds between complementary nucleotides in the different strands. Two such nucleic acid strands may be referred to as hybridized to one another.

Complementary, in the context of one sequence of nucleic acids being complementary to another sequence, refers to the ability of two strands of single-stranded nucleic acids to form hydrogen bonds between the two strands, along their length. A complementary strand can form hydrogen bonds between each corresponding nucleic acid within the strands or can form hydrogen bonds between the majority (e.g., 50%, 60%, 70%, 80%, 90%, 95%, 98%, or 100%) of corresponding nucleic acids such that the two strands hybridize. Sequences that are “sufficiently complementary” will hybridize under stringent hybridization conditions. They may have, for example, 95% sequence complementarity, 96% sequence complementarity, 97% sequence complementarity, 98% sequence complementarity, 99% sequence complementarity, or 100% sequence complementarity. A complementary strand of nucleic acids is generally made using another nucleic acid strand as a template. A first nucleotide that is capable of hybridizing to a second nucleotide sequence may be said to be a complement of the second nucleotide sequence.

A capture domain, specifically a capture sequence, can be located at the end of the capture probe and can include a free 3′ end that can be extended. In some examples, the capture domain may be a nucleotide sequence capable of hybridizing to an analyte that contains a complementary nucleotide sequence. In particular embodiments, the capture domain includes a nucleotide sequence that is capable of hybridizing to an analyte present in the sample contacted with the IC spatial array. The capture domain can be selected or designed to bind selectively or specifically to a target analyte by way of hybridization to a segment of the analyte. A CSP includes a sequence that hybridizes to the capture sequence, for example, under stringent hybridization conditions. A CSP includes the complementary sequence to the capture sequence.

The capture domain may be selected or designed to bind (or put more generally may be capable of binding) selectively or specifically to the particular nucleic acid, e.g., RNA it is desired to detect or analyze. For example, the capture domain may be selected or designed for the selective capture of mRNA. As is well known in the art, this may be on the basis of hybridization to the poly-A tail of mRNA. Thus, in a particular embodiment the capture sequence includes a polyT DNA oligonucleotide (polyT tail), i.e. a series of consecutive deoxythymidine residues linked by phosphodiester bonds, which is capable of hybridizing to the polyA tail of mRNA. Alternatively, the capture sequence may include nucleotides which are functionally or structurally analogous to polyT i.e., are capable of binding selectively to polyA, for example a polyU oligonucleotide or an oligonucleotide made of deoxythymidine analogues, wherein said oligonucleotide retains the functional property of binding to polyA. In particular embodiments, a homopolymer sequence is added to an mRNA molecule using a terminal transferase enzyme in order to produce a molecule having a polyA, polyT, or polyU sequence.

In particular embodiments, the analyte is mRNA and the capture sequence includes a polyT oligonucleotide which hybridizes with the mRNA polyA tail. In particular embodiments, the analyte is mRNA and the capture domain includes a polyU oligonucleotide sequence which hybridizes with the mRNA polyA tail. In particular embodiments, the analyte is mRNA and the capture domain includes a polyA oligonucleotide. In particular embodiments, the capture sequence includes a polyA sequence including a 10-50 nts polyA sequence. In particular embodiments, the capture sequence includes a polyA sequence including the sequence AAAAAAAAAAAAAAAAAAAA (SEQ ID NO: 1). In particular embodiments, the capture sequence includes a polyT sequence including a 10-50 nts polyT sequence. In particular embodiments, the capture sequence includes a polyT sequence including the sequence TTTTTTT TTTTTTT (SEQ ID NO: 2). In particular embodiments, the capture sequence includes a polyU sequence including a 10-50 nts polyU sequence. In particular embodiments, the capture sequence includes a polyU sequence including the sequence UUUUUUUUUUUUUUUUUUUU (SEQ ID NO: 3).

In particular embodiments, random sequences, e.g., random hexamers or similar sequences, can be used to form all or a part of the capture domain. Example, random sequences can be used in conjunction with polyT (or polyT analogue) sequences. Thus, where a capture domain includes a polyT (or a “polyT-like”) oligonucleotide, it can also include a random oligonucleotide sequence (e.g., “polyT-random sequence” probe). This can, for example, be located at 5′ or 3′ of the polyT sequence, e.g., at the 3′ end of the capture domain. The polyT-random sequence probe can facilitate the capture of the mRNA polyA tail. In particular embodiments, the capture domain can be an entirely random sequence. In particular embodiments, degenerate or partially known, partially degenerate capture domains can be used for spatial Hi-C profiling.

The capture domain may be capable of binding selectively to a desired sub-type or subset of nucleic acid, e.g., RNA, for example a particular type of RNA, or to a particular subset of a given type of RNA, for example, a particular mRNA species e.g., corresponding to a particular gene or group of genes. Such a capture domain may be selected or designed based on sequence of the RNA it is desired to capture. Thus it may be a sequence-specific capture domain, specific for a particular RNA target or group of targets. Thus, it may be based on a particular gene sequence or particular motif sequence or common/conserved sequence etc., according to principles well known in the art. Subsets of RNA include mRNA, rRNA, tRNA, SRP RNA, tmRNA, snRNA, snoRNA, SmY RNA, scaRNA, gRNA, RNase P, RNase MRP, TERC, SL RNA, aRNA, cis-NAT, crRNA, lncRNA, miRNA, piRNA, siRNA, shRNA, tasiRNA, rasiRNA, 7SK, eRNA, ncRNA or other types of RNA.

The capture domain can bind selectively to an engineered recognition sequence. In particular embodiments, the sample is modified such that analytes or target molecules are tagged. The tag is designed to target analytes and/or target molecules and, by any means known to one skilled in the art, add an engineered recognition sequence to the analyte and/or target molecule. When the analytes from the sample are transferred to the IC spatial array, the engineered recognition sequence on the analyte will bind to the capture sequence. In particular embodiments, a target protein can be tagged. In particular embodiments, target DNA can be tagged. In particular embodiments, target RNA can be tagged. In particular embodiments, target DNA is tagged to include a polyA sequence and the capture sequence includes a polyT sequence. In particular embodiments, target DNA is tagged to include a polyT sequence and the capture sequence includes a poly A sequence. In particular embodiments, the target protein is tagged using an antibody that carries a DNA sequence that hybridizes with the capture sequence.

The capture domain can be based on a gene sequence, a motif sequence or common/conserved sequence that it is designed to capture (i.e., a sequence-specific capture domain). Thus, the capture domain can be capable of binding selectively to a desired sub-type or subset of nucleic acid, for example a type or subset of mRNA. In particular embodiments, a capture domain includes an “anchoring sequence,” which is a sequence of nucleotides designed to ensure that the capture domain hybridizes to the intended mRNA. The anchor sequence can include a sequence of nucleotides, including a 1-mer, 2-mer, 3-mer or longer sequence. The sequence can be random. For example, a capture sequence including a polyT sequence can be designed to capture an mRNA. An anchoring sequence can include a random 3-mer (e.g., ACG or GGG) that helps ensure that the polyT capture domain hybridizes to an mRNA. In particular embodiments, an anchoring sequence can be VN, N, or NN, wherein N is A, T, C, or G and V is A, C, or G. Alternatively, the sequence can be designed using a specific sequence of nucleotides. In particular embodiments, the anchor sequence is at the 3′ end of the capture domain. In particular embodiments, the anchor sequence is at the 5′ end of the capture domain.

In particular embodiments, the capture sequence may prime an extension (polymerase) reaction to generate a polynucleotide that hybridizes to the captured nucleotide molecules.

In particular embodiments, the capture domain includes at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides. In particular embodiments, the capture domain includes the sequence Aₙ, wherein n is an integer of 5, 6, 7, 8, 9, 10, 11, 12, 13, 14, 15, 16, 17, 18, 19, 20, 21, 22, 23, 24, 25, 26, 27, 28, 29 or 30. In particular embodiments, the capture domain includes the sequence Tn, wherein n is an integer of 5, 6, 7, 8, 9, 10, 11, 12, 13, 14, 15, 16, 17, 18, 19, 20, 21, 22, 23, 24, 25, 26, 27, 28, 29 or 30. In particular embodiments, the capture domain includes the sequence Un, wherein n is an integer of 5, 6, 7, 8, 9, 10, 11, 12, 13, 14, 15, 16, 17, 18, 19, 20, 21, 22, 23, 24, 25, 26, 27, 28, 29 or 30.
(i-d) Other Functional Sequences.

In particular embodiments, a capture probe can include a cleavage domain. The cleavage domain represents the portion of the probe that is used to reversibly attach the capture probe to the matrix substrate. Further, one or more segments or regions of the capture probe can optionally be released from the matrix substrate by cleavage of the cleavage domain. As an example spatial barcodes and/or universal molecular identifiers (UMIs) can be released by cleavage of the cleavage domain.

In particular embodiments, the cleavage domain linking the capture probe to a matrix substrate is a covalent bond capable of cleavage by an enzyme. An enzyme can be added to cleave the cleavage domain, resulting in release of the capture probe from the capture spot. As another example, heating can also result in degradation of the cleavage domain and release of the attached capture probe from the matrix substrate. In particular embodiments, laser radiation is used to heat and degrade cleavage domains of capture probes at specific locations. In particular embodiments, the cleavage domain is a photo-sensitive chemical bond (e.g., a chemical bond that dissociates when exposed to light such as ultraviolet light). In particular embodiments, the cleavage domain can be an ultrasonic cleavage domain. For example, ultrasonic cleavage can depend on nucleotide sequence, length, pH, ionic strength, temperature, and the ultrasonic frequency (e.g., 22 kHz, 44 kHz) (Grokhovsky, 2006, Specificity of DNA cleavage by ultrasound, Molecular Biology, 40 (2), 276-283).

Other examples of cleavage domains include labile chemical bonds such as ester linkages (e.g., cleavable with an acid, a base, or hydroxylamine), a vicinal diol linkage (e.g., cleavable via sodium periodate), a Diels-Alder linkage (e.g, cleavable via heat), a sulfone linkage (e.g, cleavable via a base), a silyl ether linkage (e.g, cleavable via an acid), a glycosidic linkage (e.g, cleavable via an amylase), a peptide linkage (e.g, cleavable via a protease), or a phosphodiester linkage (e.g, cleavable via a nuclease (e.g, DNAase)).

In particular embodiments, the cleavage domain includes a sequence that is recognized by one or more enzymes capable of cleaving a nucleic acid molecule, e.g, capable of breaking the phosphodiester linkage between two or more nucleotides. A bond can be cleavable via other nucleic acid molecule targeting enzymes, such as restriction enzymes (e.g, restriction endonucleases). For example, the cleavage domain can include a restriction endonuclease (restriction enzyme) recognition sequence. Restriction enzymes cut double-stranded or single stranded DNA at specific recognition nucleotide sequences known as restriction sites. In particular embodiments, a rare-cutting restriction enzyme, i.e., enzymes with a long recognition site (at least 8 base pairs in length), is used to reduce the possibility of cleaving elsewhere in the capture probe.

In particular embodiments, the capture probe can include one or more Unique Molecular Identifiers (UMIs). A unique molecular identifier is a contiguous nucleic acid segment or two or more non-contiguous nucleic acid segments that function as a label or identifier for a particular analyte, or for a capture probe that binds a particular analyte (e.g., via the capture domain). Further details of UMIs that can be used with the systems and methods of the present disclosure are described in U.S. patent application Ser. No. 16/992,569 entitled “Systems and Methods for Using the Spatial Distribution of Haplotypes to Determine a Biological Condition,” filed Aug. 13, 2020, and PCT publication 202020176788A1 entitled “Profiling of biological analytes with spatially barcoded oligonucleotide arrays”.

In particular embodiments, a nucleotide sequence may act as a spacer to provide space between other functional domains.

Other functional domains can include nucleotide sequences that are used downstream during analysis. Further details of functional domains that can be used in conjunction with the present disclosure are described in U.S. patent application Ser. No. 16/992,569 entitled “Systems and Methods for Using the Spatial Distribution of Haplotypes to Determine a Biological Condition,” filed Aug. 13, 2020, as well as PCT publication 202020176788A1 entitled “Profiling of biological analytes with spatially barcoded oligonucleotide arrays”.
(ii) Template Array. The present disclosure describes an improvement to a template array. A typical template array includes an arrayed series of microscopic spots of oligonucleotides (hundreds of thousands of spots, generally tens of thousands, can be incorporated on a single array). The distinct position of each nucleic acid (oligonucleotide) spot (each species of oligonucleotide/nucleic acid molecule) is known as a “feature” (each species of probe may be viewed as a specific feature of the array; each feature occupies a distinct position on the array), and typically each separate feature contains in the region of picomoles (10-12 moles) of a specific DNA sequence (a “species”), which are known as “capture probes” but are herein referred to as “template probes”. Typically, these can be a short section of a gene or other nucleic acid element to which an analyte can hybridize under high-stringency hybridization conditions. In standard template arrays, the template probes are attached to a solid surface or substrate by a covalent bond to a chemical matrix, e.g., epoxy-silane, amino-silane, lysine, polyacrylamide etc.

The template probes (referred to in this section as probes) may be attached to the template array by any suitable means. The probes are immobilized to the substrate of the array by chemical immobilization. This may be an interaction between the substrate (support material) and the probe based on a chemical reaction. Such a chemical reaction typically does not rely on the input of energy via heat or light, but can be enhanced by either applying heat, e.g., a certain optimal temperature for a chemical reaction, or light of certain wavelength. For example, a chemical immobilization may take place between functional groups on the substrate and corresponding functional elements on the probes. Such corresponding functional elements in the probes may either be an inherent chemical group of the probe, e.g. a hydroxyl group or be additionally introduced. An example of such a functional group is an amine group. Typically, the probe to be immobilized includes a functional amine group or is chemically modified in order to include a functional amine group. Means and methods for such a chemical modification are well known.

The localization of said functional group within the probe to be immobilized may be used in order to control and shape the binding behavior and/or orientation of the probe, e.g. the functional group may be placed at the 5′ or 3′ end of the probe or within the sequence of the probe. A typical substrate for a probe to be immobilized includes moieties which are capable of binding to the functionalized probes, e.g. to amine-functionalized nucleic acids. Examples of such substrates are carboxy, aldehyde or epoxy substrates. Such materials are known to the person skilled in the art. Functional groups, which impart a connecting reaction between functionalized probes and array substrates are known to the person skilled in the art.

Alternative substrates on which probes may be immobilized may have to be chemically activated, e.g. by the activation of functional groups, available on the array substrate. The term “activated substrate” relates to a material in which interacting or reactive chemical functional groups were established or enabled by chemical modification procedures as known to the person skilled in the art. For example, a substrate including carboxyl groups has to be activated before use. Furthermore, there are substrates available that contain functional groups that can react with specific moieties already present in the nucleic acid probes.

Alternatively, the probes may be synthesized on the substrate. Suitable methods for such an approach are known to the person skilled in the art. Examples are manufacture techniques developed by Agilent Inc., Affymetrix Inc., Roche Nimblegen Inc. or Flexgen BV. Typically, lasers and a set of mirrors that specifically activate the spots where nucleotide additions are to take place are used. Such an approach may provide, for example, spot sizes (i.e. features) of around 30 μm or larger.

The probes on a template array may be immobilized, i.e. attached or bound, to the array preferably via the 5′ or 3′ end, depending on the chemical matrix of the array.

The covalent linkage used to couple a nucleic acid probe to an array substrate may be viewed as both a direct and indirect linkage, in that although the probe is attached by a “direct” covalent bond, there may be a chemical moiety or linker separating the “first” nucleotide of the nucleic acid probe from the, e.g. glass or silicon, substrate i.e. an indirect linkage.

The probes of the template array may be immobilized on, or interact with, the array directly or indirectly. Thus the probes need not bind directly to the array, but may interact indirectly, for example by binding to a molecule which itself binds directly or indirectly to the array (e.g., the probes may interact with (e.g., bind or hybridize to) a binding partner for the probes, i.e. a surface molecule, which is itself bound to the array directly or indirectly). In particular embodiments, the probes will be, directly or indirectly (by one or more intermediaries), bound to, or immobilized on, the array.

The substrate therefore may be any suitable substrate known to the person skilled in the art. The substrate may have any suitable form or format, e.g. it may be flat, curved, e.g. convexly or concavely curved towards the area where the interaction between the tissue sample and the substrate takes place. In particular embodiments, the substrate is a flat, i.e. planar, chip or slide.

Typically, the substrate is a solid support and thereby allows for an accurate and traceable positioning of the probes on the substrate. An example of a substrate is a solid material or a substrate including functional chemical groups, e.g. amine groups or amine-functionalized groups. In particular embodiments, the substrate is a non-porous substrate. Preferred non-porous substrates are glass, silicon, poly-L-lysine coated material, nitrocellulose, polystyrene, cyclic olefin copolymers (COCs), cyclic olefin polymers (COPs), polypropylene, polyethylene and polycarbonate.

Any suitable material known to the person skilled in the art may be used. Typically, glass or polystyrene is used. Polystyrene is a hydrophobic material suitable for binding negatively charged macromolecules because it normally contains few hydrophilic groups. For nucleic acids immobilized on glass slides, it is furthermore known that by increasing the hydrophobicity of the glass surface the nucleic acid immobilization may be increased. Such an enhancement may permit a relatively more densely packed formation. In addition to a coating or surface treatment with poly-L-lysine, the substrate, in particular glass, may be treated by silanation, e.g. with epoxy-silane or amino-silane or by silynation or by a treatment with polyacrylamide.

A number of standard arrays are commercially available, and both the number and size of the features may be varied. The arrangement of the features may be altered to correspond to the size and/or density of the cells present in different tissues or organisms. For instance, animal cells typically have a cross-section in the region of 1-100 μm, whereas the cross-section of plant cells typically may range from 1-10000 μm. Hence, Nimblegen® (Roche Diagnostics GmbH, Mannheim, Germany) arrays, which are available with up to 2.1 million features, or 4.2 million features, and feature sizes of 13 micrometers, may be preferred for tissue samples from an animal or fungus, whereas other formats may be sufficient for plant tissue samples. Commercial arrays are also available or known for use in the context of sequence analysis and in particular in the context of next-generation sequencing (NGS) technologies. In addition to commercially available arrays, which can themselves be customized, it is possible to make custom or non-standard “in-house” arrays and methods for generating arrays are well-established.

The probes (e.g., template probes) are described in more detail in section (i) Probes.
(iii) Array Features. An array includes any one, two, or three dimensional arrangement of addressable regions bearing a particular chemical moiety or moieties (e.g., capture probes or template probes) associated with that region. An array is addressable in that it has multiple regions of different molecules such that a region (a “discrete area” or “discrete volume”, collectively referred to herein as a “feature”) at a particular predetermined location (or address) on the array will have a common property. Herein, a feature refers to the addressable area or volume on the array in which the molecules share a common property. In other words, a feature refers to a discrete area when referring to a template array and a discrete volume when referring to a three dimensional array (e.g., IC spatial array). In particular embodiments, the features are not separated by a physical structure. In particular embodiments, the features are separated by a physical structure such as in a capillary array. In particular embodiments, the physical structure can be any suitable material including glass, polystyrene, silicon, polypropylene, polyethylene, polycarbonate, or combinations thereof.

In particular embodiments, it may be useful to describe an array of features by functional aspects, for example, the number of reads that can be carried out per feature (which can be a proxy for sequencing saturation), the number of transcripts that can be detected per feature, or the number of genes that can be detected per feature. For example, in particular embodiments, the number of reads that can be performed per feature is between 50,000 and 1,000,000. For example, the number of reads that can be performed per feature can be between 50,000 and 100,000, 50,000 and 150,000, 50,000 and 200,000, 50,000 and 250,000, 50,000 and 300,000, 50,000 and 350,000, 50,000 and 400,000, 50,000 and 500,000, 50,000 and 550,000, 50,000 and 600,000, 50,000 and 650,000, 50,000 and 700,000, 50,000 and 750,000, 50,000 and 800,000, 50,000 and 850,000, 50,000 and 900,000, 50,000 and 950,000, 50,000 and 1,000,000, 100,000 to 500,000, 150,000 to 500,000, 200,000 to 500,000, 250,000 to 500,000, 300,000 and 500,000, 350,000 and 500,000, 400,000 and 500,000, 450,000 and 500,000, 150,000 to 250,000, or 300,000 to 400,000. In particular embodiments, the number of reads that can be performed per feature is 70,000. In particular embodiments, the number of reads that can be performed per feature is 170,000. In particular embodiments, the number reads that can be performed per feature is 330,000. In particular embodiments, the number reads that can be performed per feature is 500,000. In particular embodiments, the number reads that can be performed per feature is 800,000.

In particular embodiments, the number of transcripts that can be detected per feature is between 20,000 and 20,000,000. For example, in particular embodiments, the number of transcripts that can be detected per feature can be between 20,000 and 30,000, 20,000 and 40,000, 20,000 and 50,000, 20,000 and 100,000, 20,000 and 500,000, 500,000 and 10,000,000, or 10,000,000 and 20,000,000. In particular embodiments, the number of transcripts that can be detected per feature is 40,000. In particular embodiments, the number of transcripts that can be detected per feature is 100,000. In particular embodiments, the number of transcripts that can be detected per feature is 5,000,000. In particular embodiments, the number of transcripts that can be detected per feature is 20,000,000.

In particular embodiments, the number of genes that can be detected per feature is between 1,000 and 5,000. For example, the number of genes that can be detected per feature can be between 1,000 and 1,500, 1,000 and 2,000, 1,000 and 2,500, 1,000 and 3,000, 1,000 and 3,500, 1,000 and 4,000, 1,000 and 4,500, 1,500 and 5,000, 2,000 and 5,000, 2,500 and 5,000, 3,000 and 5,000, 3,500 and 5,000, 4,000 and 5,000, 4,500 and 5,000, 1,500 and 2,500, 2,500 and 3,500, or 3,500 and 4,000. In particular embodiments, the number of genes that can be detected per feature is 2,000. In particular embodiments, the number of genes that can be detected per feature is 3,000. In particular embodiments, the number of genes that can be detected per feature is 4,000.

In particular embodiments, the number of proteins that can be detected per feature is between 1,000 and 5,000. For example, the number of proteins that can be detected per feature can be between 1,000 and 1,500, 1,000 and 2,000, 1,000 and 2,500, 1,000 and 3,000, 1,000 and 3,500, 1,000 and 4,000, 1,000 and 4,500, 1,500 and 5,000, 2,000 and 5,000, 2,500 and 5,000, 3,000 and 5,000, 3,500 and 5,000, 4,000 and 5,000, 4,500 and 5,000, 1,500 and 2,500, 2,500 and 3,500, or 3,500 and 4,000. In particular embodiments, the number of proteins that can be detected per feature is 2,000. In particular embodiments, the number of proteins that can be detected per feature is 3,000. In particular embodiments, the number of proteins that can be detected per feature is 4,000.

In particular embodiments, it may be useful to describe an array of features by functional aspects, for example, the number of capture probes per feature. In particular embodiments, the number of capture probes per feature includes at least 100,000; at least 200,000; at least 300,000; at least 400,000; at least 500,000; at least 600,000; at least 700,000; at least 800,000; at least 900,000; or at least 1,000,000 capture probes per feature. In particular embodiments, the number of capture probes per feature includes at least 500,000 capture probes per feature.

In particular embodiments, it may be useful to describe an array of features by functional aspects, for example, the number of UMI counts per feature. For example, in particular embodiments, the number of UMI counts that can be performed per feature is between 1,000 and 50,000. In particular embodiments, the number of UMI counts can be averaged to obtain a mean UMI per feature. In particular embodiments, the number of UMI counts can be averaged to obtain a median UMI count per feature. For example, the median UMI count per feature can be between 1,000 and 50,000, 1,000 and 40,000, 1,000 and 30,000, 1,000 and 20,000, 1,000 and 10,000, 1,000 and 5,000. In particular embodiments, the median UMI count per feature is 5,000. In particular embodiments, the median UMI count per feature is 10,000.

These components can be used to determine the sequencing saturation of the array. The sequencing saturation can be a measure of the library complexity and sequencing depth. For example, different cell types will have different amounts of analyte, thus different number of transcripts, influencing library complexity. Additionally, sequencing depth is related to the number of sequencing reads. In particular embodiments, the inverse of sequencing saturation is the number of additional reads it would take to detect a new transcript. One way of measuring the sequencing saturation of an array is to determine the number of reads to detect a new UMI. For example, if a new UMI is detected every 2 reads of the feature, the sequencing saturation would be 50%. As another example, if a new UMI is detected every 10 reads of a feature, the sequencing saturation would be 90%.

Arrays of spatially varying resolution can be implemented in a variety of ways. Herein, the resolution refers to the number of features per square area. In particular embodiments, a high resolution array of features is able to resolve parts of a cell. In particular embodiments, for example, the pitch between adjacent features in the array varies continuously along one or more linear and/or angular coordinate directions. Thus, for a rectangular array, the spacing between successive rows of features, between successive columns of features, or between both successive rows and successive columns of features, can vary continuously.

In particular embodiments, arrays of spatially varying resolution can include discrete domains with populations of features. Within each domain, adjacent features can have a regular pitch. Thus, for example, an array can include a first domain within which adjacent features are spaced from one another along linear and/or angular coordinate dimensions by a first set of uniform coordinate displacements, and a second domain within which adjacent features are spaced from one another along linear and/or angular coordinate dimensions by a second set of uniform coordinate displacements. The first and second sets of displacements differ in at least one coordinate displacement, such that adjacent features in the two domains are spaced differently, and the resolution of the array in the first domain is therefore different from the resolution of the array in the second domain.

In particular embodiments, the pitch of array features can be sufficiently small such that array features are effectively positioned continuously or nearly continuously along one or more array dimensions, with little or no displacement between array features along those dimensions. For example, in a feature array where the features correspond to regions of a substrate, the displacement between adjacent oligonucleotides can be very small-effectively, the molecular width of a single oligonucleotide. In such embodiments, each oligonucleotide can include a distinct spatial barcode such that the spatial location of each oligonucleotide in the array can be determined during sample analysis. Arrays of this type can have very high spatial resolution, but may only include a single oligonucleotide corresponding to each distinct spatial location in a sample. Therefore, although resolution may be increased, the capture potential is lower. The capture potential refers to the ability of an array to capture target molecules by having an increased number of probes to capture target molecules.

In general, the size of the array (which corresponds to the maximum dimension of the smallest boundary that encloses all features in the array along one coordinate direction) can be selected as desired, based on criteria such as the size of the sample, the feature diameter, and the density of probes within each feature. In particular embodiments, the array can be a rectangular or square array for which the maximum array dimension along each coordinate direction is 25 mm or less. In particular embodiments, the array can be a rectangular or square array for which the maximum array dimension along each coordinate direction is 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, 11 mm or less, 10 mm or less, 9 mm or less, 8 mm or less, 7 mm or less, 6 mm or less, 5 mm or less, 4 mm or less, 3 mm or less, 2 mm or less, or 1 mm or less. Thus, for example, a square array of features can have dimensions of 25 mm by 25 mm, 15 mm by 15 mm, 10 mm by 10 mm, 8 mm by 8 mm, 7 mm by 7 mm, 5 mm by 5 mm, or be smaller than 5 mm by 5 mm. In particular embodiments, a rectangular array of features can have dimensions of 25 mm by 10 mm, 25 mm by 15 mm, 10 mm by 15 mm, 8 mm by 23 mm, 17 mm by 7 mm, or 5 mm by 2 mm. In particular embodiments, the array can be any shape.
(iv) Matrix Substrate. Herein, a matrix substrate refers to a three dimensional solid or semisolid structure and material in which molecules are or can be linked to in order to form the array of discrete volumes described herein. In particular embodiments, the matrix substrate includes the starting matrix and the final product matrix substrate (which is also referred to as the IC spatial array). The composition of the matrix substrate can be any material appropriate for PCR, transcriptomics, and/or electrophoresis. The composition of the matrix substrate can be selected by one of skill in the art depending upon the intended application of the composition. In particular embodiments, the matrix substrate is a hydrogel. In particular embodiments, the matrix substrate is a solid state membrane made of glass. In particular embodiments, the solid state membrane including glass is rigid and porous. Herein, “rigid” refers to material that is unable to bend or unable to be forced out of shape.

The term hydrogel refers to a macromolecular polymer gel including a network. Within the network, some polymer chains can optionally be cross-linked, although cross-linking does not always occur. In particular embodiments, the substrate includes a hydrogel and one or more second materials. In particular embodiments, the hydrogel is placed on top of one or more second materials. For example, the hydrogel can be pre-formed and then placed on top of, underneath, or in any other configuration with one or more second materials. In particular embodiments, hydrogel formation occurs after contacting one or more second materials during formation of the substrate. Hydrogel formation can also occur within a structure (e.g., wells, ridges, projections, and/or markings) located on a substrate. In particular embodiments, a hydrogel can be formed and then cut to size.

In particular embodiments, a hydrogel can include hydrogel subunits. The hydrogel subunits can include any convenient hydrogel subunits, such as acrylamide, bis-acrylamide, polyacrylamide and derivatives thereof, polyethylene glycol) and derivatives thereof (e.g., PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, or combinations thereof. In particular embodiments, the matrix substrate is a polyacrylamide gel.

In particular embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733.

In particular embodiments, cross-linkers and/or initiators are added to hydrogel subunits. Examples of cross-linkers include bis-acrylamide and diazirine. Examples of initiators include azobisisobutyronitrile (AIBN), riboflavin, and L-arginine. Inclusion of cross-linkers and/or initiators can lead to increased covalent bonding between interacting biological macromolecules in later polymerization steps.

The matrix substrate can be a homopolymeric, a copolymeric, or a multipolymer interpenetrating polymeric hydrogel.

In particular embodiments, some hydrogel subunits are polymerized (e.g., undergo “formation”) covalently or physically cross-linked, to form a hydrogel network. For example, hydrogel subunits can be polymerized by any method including thermal crosslinking, chemical crosslinking, physical crosslinking, ionic crosslinking, photo-crosslinking, free-radical initiation crosslinking, an addition reaction, condensation reaction, water-soluble crosslinking reactions, irradiative crosslinking (e.g., x-ray, electron beam), or combinations thereof. Techniques such as lithographic photopolymerization can also be used to form hydrogels. In particular embodiments, the matrix substrate is a polyacrylamide gel and the anchoring incudes a 5′ acrydite to crosslink to the polyacrylamide gel.

In particular embodiments, a starting matrix substrate includes a matrix substrate with anchored PDEs. In particular embodiments, a starting matrix substrate includes anchored CSPs. In particular embodiments, a starting matrix substrate includes free CSPs.

In particular embodiments, an IC spatial array includes capture probes anchored to the matrix substrate.

The matrix substrate can be any shape or size that is reasonable to conduct the methods described herein. For example, the matrix substrate can be a rectangular prism, cube, cylinder or sphere. The matrix substrate dimensions may be similar to the array dimensions because the array is within the matrix substrate. A matrix substrate having a cube or rectangular prism shape has two dimensions within a plane referred to as length and width which forms a square or rectangle and a third dimension referred to as a thickness. As such, a matrix substrate having a cube or rectangular prism shape can have a length or width dimension of 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, 11 mm or less, 10 mm or less, 9 mm or less, 8 mm or less, 7 mm or less, 6 mm or less, 5 mm or less, 4 mm or less, 3 mm or less, 2 mm or less, or 1 mm or less. For example, the planar dimensions can be 25 mm by 25 mm, 24 mm by 15 mm, 23 mm by 18 mm, 20 mm by 20 mm, 18 mm by 15 mm, 13 mm by 10 mm, 7 mm by 4 mm, 4 mm by 23 mm, or 2 mm by 1 mm. In particular embodiments, the thickness of a matrix substrate shaped as a cube or rectangular prism is 10 μm-2 mm thick. In particular embodiments, the thickness of a matrix substrate is 10 μm, 15 μm, 50 μm, 100 μm, 500 μm, 900 μm, 1 mm, 1.2 mm, 1.4 mm, 1.5 mm, 1.8 mm, or 2 mm.

A matrix substrate having a cylinder shape forms, within a plane, a circle having a diameter which is projected into a third dimension a given thickness. In particular embodiments, the diameter is 25 mm or less, 24 mm or less, 23 mm or less, 22 mm or less, 21 mm or less, 20 mm or less, 19 mm or less, 18 mm or less, 17 mm or less, 16 mm or less, 15 mm or less, 14 mm or less, 13 mm or less, 12 mm or less, 11 mm or less, 10 mm or less, 9 mm or less, 8 mm or less, 7 mm or less, 6 mm or less, 5 mm or less, 4 mm or less, 3 mm or less, 2 mm or less, or 1 mm or less. In particular embodiments, the thickness of a matrix substrate with a cylinder shape is 10 μm-2 mm thick. In particular embodiments, the thickness of a matrix substrate is 10 μm, 15 μm, 50 μm, 100 μm, 500 μm, 900 μm, 1 mm, 1.2 mm, 1.4 mm, 1.5 mm, 1.8 mm, or 2 mm.

A matrix substrate having a sphere shape has a diameter that is projected in all three dimensions. In particular embodiments, the diameter is 10 μm to 25 mm.

In particular embodiments, the matrix substrate is suitable for electrophoresis. A matrix substrate that is suitable for electrophoresis means that it does not degrade under an electric field and allows for the migration of analytes under an electric field. Examples of a matrix substrate that is suitable for electrophoresis include an agarose gel or a polyacrylamide gel.

In particular embodiments, the matrix substrate can be a homogenous material. In particular embodiments, the matrix substrate can be divided into discrete volumes and each discrete volume can be separated by a physical structure such as in a capillary array.
(v) Projection into Increased Capture Spatial Array. This disclosure further describes projecting oligonucleotides of a template array into a matrix substrate to form an IC spatial array. In particular embodiments, the oligonucleotides are projected into the matrix substrate using PCR.

The polymerase chain reaction (PCR) is well known in the art, being described in U.S. Pat. Nos. 4,683,202; 4,683,195; 4,800,159; 4,965,188 and 5,512,462. During DNA synthesis and PCR, a primer acts as the starting point in which the primer binds its hybridizable (e.g., complementary) sequence within a template sequence and has an available 3′ hydroxyl group to which a transcriptase or polymerase can add additional nucleotides that hybridize to corresponding nucleotides in the template sequence, to synthesize a nucleic acid strand in the 3′ to 5′ direction. In representative PCR amplification reactions, the reaction mixture includes a template nucleotide sequence, e.g. DNA or RNA molecules on the template array, which are combined with one or more primers that are employed in the primer extension reaction, e.g., the PCR primers that hybridize to the first and/or second amplification domains (such as forward and reverse primers employed in geometric (or exponential) amplification or a single primer employed in a linear amplification). In particular embodiments, the CSP in the starting matrix substrate acts as a primer for the template nucleotide sequence on the template array.

The oligonucleotide primers will be of sufficient length to provide for hybridization to complementary template nucleotide sequence under annealing conditions. The length of the primers will depend on the length of the amplification domains, but will generally be at least 10 bp in length, at least 15 bp in length, or at least 16 bp in length. The length of primers can be as long as 30 bp in length or longer. The length of the primers can range from 18 to 50 bp in length or from 20 to 35 bp in length. The template nucleotide sequence may be contacted with a single primer or a set of two primers (forward and reverse primers), depending on whether primer extension, linear or exponential amplification of the template nucleotide sequence (template probe sequence) is desired.

In particular embodiments, a starting matrix substrate is placed on a template array in PCR-enabling media, wherein the starting matrix substrate includes PDEs and CSPs, wherein the PDEs are nucleotide sequences anchored to the matrix substrate. The template array includes a substrate including an array with a plurality of discrete areas, wherein the discrete areas form a 2-dimensional spatially-defined map (e.g., grid) and each discrete area includes a plurality of template probes immobilized to the substrate such that each template probes has a free end and each template probes is a nucleotide sequence including (from the 5′ to 3′ end): a PDET, a barcode template including a nucleotide sequence unique to the discrete area, and a CST. In particular embodiments, the CST hybridizesto the CSP. In particular embodiments, the PCR-enabling media includes PCR master mix and DNA polymerase. During a PCR cycle, temperature is increased and decreased to amplify, or copy a provided nucleotide sequence. When the mixture is cooled, the CSP migrates to and anneals with the CST (FIG. 1C). At this point, the DNA polymerase synthesizes new strands of DNA starting at the annealed capture domains, producing a sequence that hybridizes to the template probe that is herein referred to as a bridge probe (FIG. 1D). In particular embodiments, the bridge probe includes a bridge PDE, a sequence that hybridizes to the barcode referred to herein as a bridge barcode (or complementary barcode or barcode′), and the CSP. In particular embodiments, the bridge PDE hybridizes to the PDE anchored to the matrix substrate and therefore hybridizes with the anchored PDE (FIG. 1E), resulting in the bridge probe linked to the matrix substrate (FIG. 1F) via the PDE and bridge PDE. During another PCR cycle, the anchored PDEs are extended through the bridge probe to create a capture probe (FIG. 1G) and the bridge probe will dehybridize (FIG. 1H). In particular embodiments, the capture probe hybridizes to the bridge probe and identical to the template probe. In particular embodiments, the capture probe includes a PDE, a barcode, and a capture sequence. In particular embodiments, the majority (e.g., 50%, 60%, 70%, 80%, 90%, 95%, 98%, or 100%) of capture probes include a PDE, a barcode, and a capture sequence. In subsequent PCR cycles, the bridge probes hybridize with remaining anchored PDEs within the matrix substrate and extension through the bridge probe to create additional capture probes. This results in an IC spatial array including an array and a thickness, wherein the matrix substrate includes a plurality of discrete volumes, each discrete volume occupying a distinct x and y position on the array and continuing through the thickness of the matrix substrate in the z direction such that each discrete volume occupies a discrete volume within the matrix substrate, each discrete volume includes a plurality of capture probes anchored to the matrix substrate such that the capture probes have a free end, wherein the capture probes each include: a barcode including a nucleotide sequence unique to the discrete volume in which the capture probe is anchored; and a capture sequence. In particular embodiments, the majority (e.g., 50%, 60%, 70%, 80%, 90%, 95%, 98%, or 100%) of capture probes have a free end. In particular embodiments, the majority (e.g., 50%, 60%, 70%, 80%, 90%, 95%, 98%, or 100%) of template probes have a free end.

“PCR-enabling media” refers to a composition of materials needed to perform PCR or amplify segments of DNA. As an example, of a PCR-enabling media, PCR master mix includes DNA polymerase, dNTPs, cofactors, and buffer. In particular embodiments, the cofactor includes MgCL2. In particular embodiments, a PCR master mix can include PCR buffer, dNTPs, forward primer, reverse primer, Taq DNA polymerase, template DNA, cofactors, and/or water. Many premade or commercially available PCR master mixes are available. Premade and commercially available PCR master mixes may require preparation such as thawing, chilling, dilution with PCR-grade water, and mixing. Examples of commercially available PCR master mixes include ReadyMix™ Taq PCR Reaction Mix, Roche PCR Master, REDTaq® (Merck KGAA, Darmstadt, Germany) ReadyMix™ PCR Reaction Mix, Millipore KOD Hot Start Master Mix, KiCqStart® (Qiagen Beverly, Inc., Beverly, MA) One-Step Probe RT-qPCR ReadyMix™, MystiCq® (Qiagen Beverly Inc.) microRNA cDNA Synthesis Mix, FastStart TaqMan® (Roche Molecular Systems, Inc., Pleasanton, CA) Probe Master, and Roche EagleTaq Universal Master Mix (ROX).

By projecting the template array into a format with volume (e.g., matrix substrate), the capture probes per feature of the array is no longer limited by the surface area of the two-dimensional substrate. Rather, a three-dimensional matrix substrate can include a similar density of capture probes in a single two-dimensional plane as its two-dimensional counterpart. Furthermore, the three-dimensional matrix substrate can include that density of capture probes in several planes at varying depths (3^{rd} dimension) of the matrix substrate. This gives the array projected into a matrix substrate a higher capture potential compared to the template array because there are more capture probes available within the same sized discrete volume.

In particular embodiments, the projection of the barcoded capture probes into the matrix substrate requires extension. Extension refers to any method where two nucleic acid sequences become linked (e.g., hybridized) by an overlap of their respective terminal complementary nucleic acid sequences. Such linking can be followed by nucleic acid extension (e.g., an enzymatic extension) of one, or both termini using the other nucleic acid sequence as a template for extension. Enzymatic extension can be performed by an enzyme including a polymerase and/or a reverse transcriptase.

Ligation refers to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. In particular embodiments, ligations are carried out enzymatically to form a phosphodiester linkage between a 5′ carbon terminal nucleotide of one oligonucleotide with a 3′ carbon of another nucleotide. In particular embodiments, ligations are carried out chemically (e.g., using click chemistry).

A nucleic acid extension involves the incorporation of one or more nucleic acids (e.g., A, G, C, T, U, nucleotide analogs, or derivatives thereof) into a molecule (such as a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer (e.g., the PDE or CSP) that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly(dT) sequence can be used as a template for single-strand synthesis of a corresponding cDNA molecule.

PCR amplification refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965, 188, and 5,512,462. In a typical PCR amplification, the reaction mixture includes the genetic material to be amplified, an enzyme, one or more primers that are employed in an extension reaction, and reagents for the reaction. The oligonucleotide primers are of sufficient length to provide for hybridization to complementary genetic material under annealing conditions. The length of the primers generally depend on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In particular embodiments, the PCR amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

Suitable examples of DNA polymerases that can be used include: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase (New England Biolabs, Inc., Ipswich, MA), LongAmp® Hot Start Taq DNA polymerase (New England Biolabs, Inc.), Crimson LongAmp® Taq DNA polymerase (New England Biolabs, Inc.), Crimson Taq DNA polymerase, OneTaq® DNA polymerase (New England Biolabs, Inc.), OneTaq® Quick-Load® DNA polymerase (New England Biolabs, Inc.), Hemo KlenTaq® DNA polymerase (New England Biolabs, Inc.), REDTaq® DNA polymerase (Merck KGAA, Germany), Phusion® DNA polymerase, Phusion® High-Fidelity DNA polymerase (Thermo Fisher Scientific Baltics, Lithuania), Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

The term DNA polymerase includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

In particular embodiments, PCR amplification can include reactions such as a strand-displacement amplification reaction, a rolling circle amplification reaction, a ligase chain reaction, a transcription-mediated amplification reaction, an isothermal amplification reaction, and/or a loop-mediated amplification reaction.

In some embodiments (e.g., when the PCR amplification amplifies captured DNA), the PCR amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include Tth DNA ligase, Taq DNA ligase, Thermococcus sp. (strain 90N) DNA ligase (9oN™ DNA ligase, available from New England Biolabs, Ipswich, Mass.), and Ampligase™ (available from Epicentre Biotechnologies, Madison, Wis.). Derivatives, e.g. sequence-modified derivatives, and/or mutants thereof, can also be used.

An amplification product refers to molecules that result from reproduction or copying of another molecule. Generally, the molecules copied or reproduced are nucleic acid molecules, specifically DNA or RNA molecules. In particular examples, the molecule reproduced or copied may be used as a template for the produced molecules. In particular embodiments, a template probe acts as a template to produce a bridge probe as the amplification product. In particular embodiments, the bridge probe acts as a template to produce a capture probe as the amplification product. In particular embodiments, an analyte captured by the capture sequence may be used as a template to produce an amplification product. Various enzymes (e.g., reverse transcriptase) may be used for this process. The cDNA amplification product may in turn act as a template for amplification that may also be called amplification products. Various enzymes (e.g., Taq polymerase) may be used for this process.

In some embodiments, genetic material is amplified by reverse transcription polymerase chain reaction (RT-PCR). The desired reverse transcriptase activity can be provided by one or more distinct reverse transcriptase enzymes, suitable examples of which include: M-MLV, MuLV, AMV, HIV, ArrayScript™, MultiScribe™ ThermoScript™ (Boehringer Mannheim Corporation, Indianapolis, IN), and SuperScript® (Life Technologies, Inc. Gaithersburg, MD) I, II, III, and IV enzymes. Reverse transcriptase includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g. M-MLV, and compositions including unmodified and modified enzymes are commercially available, e.g. ArrayScript™, MultiScribe™ ThermoScript (Boehringer Mannheim Corporation, Indianapolis, IN), and SuperScript® (Life Technologies, Inc. Gaithersburg, MD) I, II, III, and IV enzymes.

Certain reverse transcriptase enzymes (e.g., Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g. an AMV or MMLV reverse transcriptase.

In some cases the synthesized oligos will have “scars” on the backbone which could prevent polymerase from synthesizing the second strand. Those oligos still carry the correct nucleobases for synthesis, and can hybridize to a matched complementary strand. The main strategy is to hybridize the complementary strand with very slow annealing process, and then ligate multiple pieces to form the complete second strand. This method of synthesis is referred to as ligation or DNA ligation. For example, to synthesize a capture probe using ligation, a template strand and capture strand segments that are complementary to the template strand are provided (FIG. 8). In particular embodiments, the template strand includes a PDET, a sequence encoding a complementary sequence to the barcode and a CST. In particular embodiments, the sequence encoding the complementary sequence to the barcode is divided into at least two template strand segments. Each template strand segment is sufficiently complementary to a capture strand segment. In particular embodiments, the capture strand segments include: a first half of the barcode and 6 or more nucleotide bases flanking each side of the first half of the barcode, and a second half of the barcode and 6 or more nucleotide bases flanking each side of the second half of the barcode. In particular embodiments, the template strand and capture strand segments are mixed with hybridization buffer and allowed to anneal. During annealing, the first complementary strand and second complementary strand will hybridize to the template strand. After this, the DNA ligase, DNA ligase buffer, and a CSP is added to the mixture. In particular embodiments, the DNA ligase catalyzes phosphodiester bonds at single-strand breaks in DNA between the 3′OH group and 5′-monophophate to form a capture probe that is sufficiently complementary to the template sequence. DNA ligation results in formation of a capture probe. After ligation, the capture probe can be dissociated from the template strand. In particular embodiments, the capture probe a sequence complementary to the template strand. In particular embodiments, the capture probe includes a PDE, capture strand segments, and a capture sequence primer (CSP).

In particular embodiments, hybridization buffer includes any liquid that allows for complementary sequences to hybridize. In particular embodiments, hybridization buffer includes New England Biolabs buffer.

In particular embodiments, an example template strand includes the sequence CACGACGCTCTTCCGATCTCANNNNNNNACTCTCACACAANNNNNNNCACATTTTTTTTTTT TTTTTTTTT (SEQ ID NO: 10). The barcode N14 is separated into two parts, N7 and N7. In particular embodiments, the template strand and complementary strand segments are mixed in hybridization buffer. In particular embodiments, the complementary strand segments include (1) GTGCTGCGAGAAGGC (phosphate) 5′ (SEQ ID NO: 11), (2) TAGAGTNNNNNNNTGAGAG (phosphate) 5′ (SEQ ID NO: 12), and (3) TGTGTTNNNNNNNGTGTAA (phosphate) 5′ (SEQ ID NO: 13). In particular embodiments, this mixture is heated to 80° C. and very slow annealing is performed. In particular embodiments, slow annealing includes a reduction in temperature of 0.5° C./second down to 50° C. to 45° C. During this process, the complete match N7 will hybridize to the template strand. In particular embodiments, the mixture is flushed with hybridization buffer and a polyA sequence is added at 50° C. to 45° C. In particular embodiments, DNA ligase and DNA ligase buffer are added and ligation occurs at room temperature. In particular embodiments, ligation occurs overnight.

Examples of DNA ligase include T4 DNA ligase, T7 DNA ligase, Taq DNA ligase, Electroligase® (New England Biolabs Inc., Ipswich, MA), HiFi Taq DNA ligase, Hi-T4™ DNA ligase (New England Biolabs Inc.), Salt-T4R DNA ligase (New England Biolabs Inc.), SplintR® ligase (New England Biolabs Inc.). In particular embodiments, the DNA ligase includes T4 DNA ligase. In particular embodiments, the DNA ligase buffer includes T4 DNA ligase buffer (New England Biolabs Inc.).

Splitting a barcode length of n=14 nucleotides into two n=7 nucleotides in length barcodes reduces complexity during hybridization. In particular embodiments, the barcode can be split into any suitable lengths. For example, a 14 nucleotide barcode can be split into a 4 and 10, 5 and 9, 6 and 8, 7 and 7 nucleotide length sequence. Adding at least 6 flanking nucleotides on each side of the shortened barcode is beneficial because some ligases (e.g., T4 DNA ligase) needs 5-6 bases to recognize double stranded DNA. In particular embodiments, sequences including 3, 4, 5, 6, 7, 8, 9, 10, 11, or 12 nucleotides in length can be used to flank the split barcode sequence. In particular embodiments, the flanking sequence can flank one or both sides of the split barcode sequence.
(vi) Isometric Expansion. Isometric expansion methods that can be used include hydration, a preparative step in expansion microscopy, as described in Chen et al., Science 347 (6221): 543-548, 2015; Asano et al. Current Protocols. 2018, 80:1, doi: 10.1002/cpcb.56 and Gao et al. BMC Biology. 2017, 15:50, doi: 10.1186/sl2915-017-0393-3, Wassie, A. T., et al, Expansion microscopy: principles and uses in biological research, Nature Methods 16 (1): 33-41 (2018).

In particular embodiments, a sample embedded in a gel or matrix substrate can be isometrically expanded. In particular embodiments, isometric expansion can be performed on the matrix substrate.

In general, the steps used to perform isometric expansion can depend on the characteristics of the sample (e.g., thickness of tissue section, fixation, cross-linking), the characteristics of the matrix substrate, and/or the analyte of interest (e.g., different conditions to anchor RNA, DNA, and protein to a gel).

Isometric expansion can be performed by anchoring one or more components of a sample to a gel, followed by gel formation, proteolysis, and swelling. Isometric expansion of the sample can occur prior to immobilization of the sample on a substrate, or after the sample is immobilized to a substrate. In particular embodiments, the isometrically expanded sample can be removed from the substrate prior to contacting the expanded sample with a spatially barcoded array (e.g., IC spatial array or template array).

In particular embodiments, proteins or other molecules in the sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al, Nat. Methods 13:679-684, 2016).

Isometric expansion of the sample can increase the spatial resolution of the subsequent analysis of the sample. For example, isometric expansion of the biological sample can result in increased resolution in spatial profiling (e.g., single-cell profiling). The increased resolution in spatial profiling can be determined by comparison of an isometrically expanded sample with a sample that has not been isometrically expanded.

In particular embodiments, a matrix substrate and/or sample volume is isometrically expanded in all 3 dimensions, at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× in each dimension of its non-expanded size. In particular embodiments, the matrix substrate and/or sample is isometrically expanded to at least 2× and less than 20× of its non-expanded volume.
(vii) Shrinking the Array. In particular embodiments, the resolution of the IC spatial array can be further enhanced by shrinking the matrix substrate after projecting the capture probes into the matrix substrate. In particular embodiments, the matrix substrate is isometrically expanded, capture probes are projected into the matrix substrate, and then the matrix substrate is shrunk.

As used herein “shrinking” or “reducing the size” of a matrix substrate refers to any process causing the matrix substrate to physically contract and/or the size of the matrix substrate to decrease in volume. There are many methods known to one of skill in the art for shrinking or reducing the volume of a matrix substrate. Examples of a method to shrink or reduce the volume of a matrix substrate include exposing the matrix substrate to one or more of: a dehydrating solvent, a salt, heat, a vacuum, lyophilization, desiccation, filtration, air-drying, or combinations thereof. For example, the matrix substrate can be decreased in volume by removing or exchanging solvents, salts, or water (see, e.g., Long and Williams. Science. 2018; 362 (6420): 1244-1245, and Oran et al. Science 2018; 362 (6420): 1281-1285), by controlling temperature or pH (see e.g., Ahmed, E. M. J. of Advanced Research. 2015 March; 6 (2): 105-121), or by removing water.

In particular embodiments, the matrix substrate is not decreased in volume. In particular embodiments, the matrix substrate can be decreased in volume. In particular embodiments, the shrunken matrix substrate is stabilized. Examples of solvents that may be used to form a shrunken matrix substrate include a ketone, such as methyl ethyl ketone (MEK), isopropanol (IPA), acetone, 1-butanol, methanol (MeOH), dimethyl sulfoxide (DMSO), glycerol, propylene glycol, ethylene glycol, ethanol, (k) 1,4-dioxane, propylene carbonate, furfuryl alcohol, N,N-dimethylformamide (DMF), acetonitrile, aldehyde, such as formaldehyde or glutaraldehyde, or any combinations thereof.

In particular embodiments, the matrix substrate is shrunken or stabilized via a cross-linking agent. For example, the cross-linking agent may include disuccinimidyl suberate (DSS), dimethylsuberimidate (DMS), formalin, and dimethyladipimidate (DMA), dithio-bis(-succinimidyl propionate) (DSP), disuccinimidyl tartrate (DST), and ethylene glycol bis(succinimidyl succinate) (EGS).

In particular embodiments, the matrix substrate is processed with salts to form a shrunken matrix substrate. Examples of salts that may be used to form a shrunken substrate matrix are inorganic salts including aluminum, ammonium, barium, beryllium, calcium, cesium, lithium, magnesium, potassium, rubidium, sodium, and strontium salts. Further examples of inorganic salts include sodium chloride, potassium chloride, lithium chloride, cesium chloride, sodium fluoride, sodium bromide, sodium iodide, sodium nitrite, potassium sulfate, potassium nitrate, potassium carbonate, potassium bicarbonate, sodium sulfate, sodium nitrate, sodium carbonate, sodium bicarbonate, calcium sulfate, copper oxychloride, calcium chloride, calcium carbonate, calcium bicarbonate, magnesium sulfate, magnesium nitrate, magnesium chloride, magnesium carbonate, magnesium bicarbonate, ammonium sulfate, ammonium chloride, ammonium nitrate, ammonium carbonate, ammonium bicarbonate, trisodium phosphate, tripotassium phosphate, calcium phosphate, copper (II) sulfate, sodium sulfide, potassium sulfide, calcium sulfide, potassium permanganate, iron (II) chloride, iron (III) chloride, iron (2+) sulfate, iron (III) sulfate, iron (II) nitrate, iron (III) nitrate, manganese (II) chloride, manganese (III) chloride, manganese (II) sulfate, manganese (II) nitrate, zinc chloride, zinc nitrate, zinc sulfate, ammonium orthomolybdate, monopotassium phosphate, nickel (II) sulfate, nickel (II) nitrate, sodium metavanadate, sodium paravanadate, potassium dichromate, ammonium dichromate, antipyonin, ammonium nitrite, potassium fluoride, sodium fluoride, ammonium fluoride, calcium fluoride, chrome alum, potassium alum, potassium iodide, sodium hypochlorite, tin (II) sulfate, tin (II) nitrate, gold selenite, dicesium chromate, potassium perchlorate, calcium perchlorate, aluminum sulphate, lead (II) bisulfate, barium phosphate, barium hydrogen orthophosphate, barium dihydrogen phosphate, silver dichromate, potassium bromate, sodium bromate, sodium iodate, sodium silicate, diammonium phosphate, ammonium phosphate, ammonium dihydrogen phosphate, chromium orthophosphate, copper (II) chloride, copper (I) chloride, sodium tetrametaphosphate, potassium heptafluoroniobate, zinc phosphate, sodium sulfite, copper (I) nitrate, copper (II) nitrate, potassium silicate, copper (II) carbonate basic, copper (II) carbonate salts of acrylic acid and sulfopropyl acrylate.

In particular embodiments, the removal of water includes an acid. Examples of an acid include: HCl, HI, HBr, HClO₄, HClO₃, HNO₃, H₂SO₄, phosphoric acid, phosphorous acid, acetic acid, oxalic acid, ascorbic acid, carbonic acid, sulfurous acid, tartaric acid, citric acid, malonic acid, phthalic acid, barbituric acid, cinnamic acid, glutaric acid, hexanoic acid, malic acid, folic acid, propionic acid, stearic acid, trifluoroacetic acid, acetylsalicylic acid, glutamic acid, azelaic acid, benzilic acid, fumaric acid, gluconic acid, lactic acid, oleic acid, propiolic acid, rosolic acid, tannic acid, uric acid, gallic acid, and combinations of two or more thereof.

In particular embodiments, the matrix substrate is exposed to a different pH environment. For example, the matrix substrate can be exposed to an acidic pH or a basic pH. In particular embodiments, the matrix substrate is exposed to a pH of less than 6.5. In particular embodiments, the matrix substrate is exposed to a pH of 6, 5.5, 5, 4.5, 4, 3.5, 3, 2.5, 2, 1.5, or 1. In particular embodiments, the matrix substrate is exposed to a pH of greater than 7.5. In particular embodiments, the matrix substrate is exposed to a pH of 8, 8.5, 9, 9.5, 10, 10.5, 11, 11.5, 12, 12.5, 13, 13.5, or 14.

In particular embodiments, the matrix substrate undergoes an alteration in temperature (e.g., an alteration from 37° C., 38° C., 39° C., 40° C., 41° C., 42° C., 43° C., 44° C., 45° C., 46° C., 47° C., 48° C., 49° C. to 50° C., 51° C., 52° C., 53° C., 54° C., 55° C., 56° C., 57° C., 58° C., 59° C. 60° C., 61° C., 62° C., 63° C., 64° C., 65° C., 66° C., 67° C., 68° C., 69° C., 70° C., or higher, or any temperature alteration encompassed within these ranges) to form a shrunken matrix substrate.

In particular embodiments, the matrix substrate can be decreased in size in linear dimension by 2 fold, 3 fold, 4 fold, 5 fold, 6 fold, 7 fold, 8 fold, 9 fold, or any intervals therein. In particular embodiments, the matrix substrate can be decreased in volume by 1 fold, 5 fold, 10 fold, 15 fold, 20 fold, 25 fold, 30 fold, 35 fold, 40 fold, 45 fold, 50 fold, 55 fold, 60 fold, 65 fold 70 fold, 75 fold, 80 fold, or any intervals therein.

In particular embodiments, the matrix substrate can be re-expanded. In particular embodiments, matrix substrate can be isometrically re-expanded. In particular embodiments, the matrix substrate can be isometrically expanded and then decreased in size.

In particular embodiments, decreasing the volume of the matrix substrate can increase the spatial resolution of the subsequent analysis of the sample. The increased resolution in spatial profiling can be determined by comparison of spatial analysis of the sample using a shrunken matrix substrate with a non-shrunken matrix substrate.

A “shrunken array” includes a plurality of discrete volumes (or features) attached to, or embedded in, a matrix substrate that have been reduced in volume (e.g., reduction in diameter or volume). A sample can be contacted with a shrunken array and further contacted with a solution capable of rehydrating the shrunken array. In particular embodiments, analyte transfer and capture is driven by molecular diffusion. The process of rehydrating the shrunken array by providing a permeabilization solution or tissue stain to the sample can promote the transfer of analytes (e.g., transcripts) present in the sample towards the capture probes, thereby improving capture efficiency of the analytes. See, e.g., J. Vlassakis, A. E. Herr. “Effect of Polymer Hydration State on In-Gel Immunoassays.” Anal. Chem. 2015, 87 (21): 11030-8.
(viii) Method of Use.
(viii-a) Types of Samples. A sample can be any nonbiological sample, biological sample, or can be derived from any biological sample in which analysis of the sample's analytes and the analyte's position within the sample is desired. Methods and compositions disclosed herein can be used to analyze a sample, which may be obtained from a subject using any of a variety of techniques including biopsy, surgery, and laser capture microscopy (LCM), and generally includes cells and/or other biological material from the subject. Subjects include animals (e.g., humans, non-human mammals, birds, insects, fish, worms, reptiles, dogs, cats, rats, mice, etc.), plants (e.g., moss, ferns, etc.), fungi (e.g., fungus, mold, mushrooms, yeast, mildew, etc.), protists (e.g., algae, protozoans, etc.), and prokaryotes (e.g., bacteria, cyanobacteria, spirochetes, etc.). A sample can be a single cell, a plurality of cells, or a tissue. A sample from a single organism can include one or more other organisms or components therefrom. For example, a mammalian tissue section may include a prion, a viroid, a virus, a bacterium, a fungus, or components from other organisms, in addition to mammalian cells and non-cellular tissue components. Subjects from which samples can be obtained can be healthy or asymptomatic subjects, subjects that have or are suspected of having a disease (e.g., a subject with a disease such as cancer) or a pre-disposition to a disease, and/or subjects in need of therapy or are suspected of needing therapy.

The sample can include any number of macromolecules, for example, cellular macromolecules and organelles (e.g., mitochondria and nuclei). The sample can be obtained as a tissue sample, such as a tissue section, biopsy, a core biopsy, needle aspirate, or fine needle aspirate. The sample can be a fluid sample, such as a blood sample, urine sample, or saliva sample, and cells and cellular components therein may be analyzed after placing the cells or cellular components on a substrate. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

The sample may thus be a harvested or biopsied tissue sample, or possibly a cultured sample. Representative samples include clinical samples e.g. whole blood or blood-derived products, blood cells, tissues, biopsies, or cultured tissues or cells etc. including cell suspensions. Artificial tissues may for example be prepared from cell suspension (including for example blood cells). Cells may be captured in a matrix substrate (for example a gel matrix substrate e.g. agar, agarose, etc) and may then be sectioned in a conventional way. Such procedures are known in the art in the context of immunohistochemistry (see e.g. Andersson et al 2006, J. Histochem. Cytochem. 54 (12): 1413-23. Epub 2006 Sep. 6).

Samples can be derived from a homogeneous culture or population from a subject or from a collection of several different subjects, for example, in a community or ecosystem.

Samples can include one or more diseased cells. A diseased cell can have altered metabolic properties, gene expression, protein expression, and/or morphologic characteristics. Examples of diseases include inflammatory disorders, metabolic disorders, nervous system disorders, and cancer. Cancer cells can be derived from solid tumors, hematological malignancies, cell lines, or obtained as circulating tumor cells. Samples can also include fetal cells and immune cells.

Samples can include analytes (e.g., RNA, DNA, and/or protein) in a matrix substrate. In particular embodiments, amplicons (e.g., rolling circle amplification products) derived from or associated with can be embedded in a matrix substrate. In particular embodiments, a matrix substrate may include a network of natural molecules and/or synthetic molecules that are chemically and/or enzymatically linked. In particular embodiments, a matrix substrate may include a synthetic polymer.
(viii-b) Sample Preparation. A variety of steps can be performed to prepare or process a sample for and/or during an assay. Except where indicated otherwise, the preparative or processing steps described below can generally be combined in any manner and in any order to appropriately prepare or process a particular sample for analysis.
(viii-b-1) Sectioning. A sample can be harvested from a subject (e.g., via surgical biopsy, whole subject sectioning) or grown in vitro on a growth substrate or culture dish as a population of cells and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome.

The thickness of the tissue section can be a fraction of (e.g, less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g, 10-20 micrometers thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 11, 12, 13, 14, 15, 20, 30, 40, or 50 micrometers. Thicker sections can also be used if desired or convenient, e.g, at least 70, 80, 90, or 100 micrometers or more. Typically, the thickness of a tissue section is between 1-100 micrometers, 1-50 micrometers, 1-30 micrometers, 1-25 micrometers, 1-20 micrometers, 1-15 micrometers, 1-10 micrometers, 2-8 micrometers, 3-7 micrometers, or 4-6 micrometers, but as mentioned above, sections with thicknesses larger or smaller than these ranges can also be analyzed.

Multiple sections can also be obtained from a single sample. For example, multiple tissue sections can be obtained from a surgical biopsy sample by performing serial sectioning of the biopsy sample using a sectioning blade. Spatial information among the serial sections can be preserved in this manner, and the sections can be analyzed successively to obtain three-dimensional information about the sample.
(viii-b-2) Freezing. In particular embodiments, the sample can be prepared by deep freezing at a temperature suitable to maintain or preserve the integrity (e.g., the physical characteristics) of the sample structure. The frozen sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C. A sample can be snap frozen in isopentane and liquid nitrogen. Frozen samples can be stored in a sealed container prior to further sample preparation steps.
(viii-b-3) Fixation and Embedding. In particular embodiments, the sample can be prepared using formalin-fixation and paraffin-embedding (FFPE), which are established methods. In particular embodiments, cell suspensions and other non-tissue samples can be prepared using FFPE. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof. When acetone fixation is performed, pre-permeabilization steps (described below) may not be performed. Alternatively, acetone fixation can be performed in conjunction with permeabilization steps.

As an alternative to paraffin embedding described above, a biological sample can be embedded in any of a variety of other embedding materials to provide structural substrate to the sample prior to sectioning and other handling steps. In particular embodiments, the embedding material can be removed, e.g., prior to analysis of tissue sections obtained from the sample. Suitable embedding materials include waxes, resins (e.g., methacrylate resins), epoxies, and agar.

The biological sample can be embedded in a matrix substrate (e.g., a hydrogel matrix substrate). In particular embodiments, the embedding material can be applied to the sample one or more times. Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel.

The sample can be immobilized in the hydrogel via cross-linking of the polymer material that forms the hydrogel. Cross-linking can be performed chemically and/or photochemically, or alternatively by any other hydrogel-formation method known in the art.

The composition and application of the hydrogel-matrix substrate to a biological sample typically depends on the nature and preparation of the biological sample (e.g., sectioned, non-sectioned, type of fixation). As one example, where the biological sample is a tissue section, the hydrogel-matrix substrate can include a monomer solution and an ammonium persulfate (APS) initiator/tetramethylethylenediamine (TEMED) accelerator solution. As another example, where the biological sample includes cells (e.g., cultured cells or cells disassociated from a tissue sample), the cells can be incubated with the monomer solution and APS/TEMED solutions. For cells, hydrogel-matrix substrate gels are formed in compartments, including devices used to culture, maintain, or transport the cells. For example, hydrogel-matrices can be formed with monomer solution plus APS/TEMED added to the compartment to a depth ranging from 0.1 μm to 2 mm.

Additional methods and aspects of hydrogel embedding of biological samples are described for example in Chen et al., Science 347 (6221): 543-548, 2015.
(viii-b-4) Staining. To facilitate visualization, biological samples can be stained using a wide variety of stains and staining techniques. A sample can be stained using any number of stains including acridine orange, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, propidium iodide, rhodamine, or safranine.

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In particular embodiments, the sample can be stained using Romanowsky stain, including Wright's stain, Jenner's stain, Can-Grunwald stain, Leishman stain, and Giemsa stain.

Samples can also be destained. Methods of destaining or discoloring a biological sample are known in the art, and generally depend on the nature of the stain(s) applied to the sample. For example, if one or more immunofluorescent stains are applied to the sample via antibody coupling, such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65 (8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905.
(viii-b-5) Permeabilization. In particular embodiments, a sample can be permeabilized to facilitate transfer of analytes out of the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In particular embodiments, the IC spatial array is adapted to facilitate analyte migration from the sample onto the IC spatial array. In particular embodiments, a sample is permeabilized before being contacted with an IC spatial array.

In general, a sample can be permeabilized by exposing the sample to one or more permeabilizing agents. Suitable agents for this purpose include organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In particular embodiments, the detergent is an anionic detergent (e.g., SDS or N-lauroylsarcosine sodium salt solution). In particular embodiments, the sample can be permeabilized using any of the methods described herein (e.g., using any of the detergents described herein, e.g., SDS and/or N-lauroylsarcosine sodium salt solution) before or after enzymatic treatment (e.g., treatment with any of the enzymes described herein, e.g., trypin, proteases (e.g., pepsin and/or proteinase K)).

In particular embodiments, a sample can be permeabilized by exposing the sample to greater than 1.0 w/v % (e.g., greater than 2.0 w/v %, greater than 3.0 w/v %, greater than 4.0 w/v %, greater than 5.0 w/v %, greater than 6.0 w/v %, greater than 7.0 w/v %, greater than 8.0 w/v %, greater than 9.0 w/v %, greater than 10.0 w/v %, greater than 11.0 w/v %, greater than 12.0 w/v %, or greater than 13.0 w/v %) sodium dodecyl sulfate (SDS) and/or N-lauroylsarcosine or N-lauroylsarcosine sodium salt. In particular embodiments, a sample can be permeabilized by exposing the sample (e.g., for 5 minutes to 1 hour, 5 minutes to 40 minutes, 5 minutes to 30 minutes, 5 minutes to 20 minutes, or 5 minutes to 10 minutes) to 1.0 w/v % to 14.0 w/v % (e.g., 2.0 w/v % to 14.0 w/v %, 2.0 w/v % to 12.0 w/v %, 2.0 w/v % to 10.0 w/v %, 4.0 w/v % to 14.0 w/v %, 4.0 w/v % to 12.0 w/v %, 4.0 w/v % to 10.0 w/v %, 6.0 w/v % to 14.0 w/v %, 6.0 w/v % to 12.0 w/v %, 6.0 w/v % to 10.0 w/v %, 8.0 w/v % to 14.0 w/v %, 8.0 w/v % to 12.0 w/v %, 8.0 w/v % to 10.0 w/v %, 10.0% w/v % to 14.0 w/v %, 10.0 w/v % to 12.0 w/v %, or 12.0 w/v % to 14.0 w/v %) SDS and/or N-lauroylsarcosine salt solution and/or proteinase K (e.g., at a temperature of 4% to 35° C., 4° C. to 25° C., 4° C. to 20° C., 4° C. to 10° C., 10° C. to 25° C., 10° C. to 20° C., 10° C. to 15° C., 35° C. to 50° C., 35° C. to 45° C., 35° C. to 40° C., 40° C. to 50° C., 40° C. to 45° C., or 45° C. to 50° C.)

The IC spatial array is contacted with a sample, and the sample can be permeabilized through application of permeabilization reagents. Permeabilization reagents may be administered by placing the array/sample assembly within a bulk solution. Alternatively, permeabilization agents may be administered to the sample via a diffusion-resistant medium and/or a physical barrier such as a lid, where the sample is sandwiched between the diffusion-resistant medium and/or barrier and the array-containing substrate.

In particular embodiments, the sample can be incubated with a permeabilizing agent to facilitate permeabilization of the sample. In particular embodiments, the rate of permeabilization is slowed prior to contacting a sample with an IC spatial array (e.g., to limit diffusion of analytes away from their original locations in the sample). In particular embodiments, modulating the rate of permeabilization can occur by modulating the permeabilizing agents that the sample is exposed to. For example, a permeabilization agent can be provided to a sample prior to contact with an IC spatial array, wherein the permeabilization agent is inactive until a condition (e.g., temperature, pH, and/or light) is changed or an external stimulus (e.g., a small molecule, an enzyme, and/or an activating reagent) is provided.

Additional methods for sample permeabilization are described, for example, in Jamur et al., Method Mol. Biol. 588:63-66, 2010.
(viii-b-6) Analyte Enrichment. The analyte species of interest can be selectively enriched (e.g., Adiconis, et. al., Comparative analysis of RNA sequencing methods for degraded and low-input samples, Nature, vol. 10, July 2013, 623-632). For example, one or more species of analyte can be selected by addition of one or more oligonucleotides to the sample. In particular embodiments, the additional oligonucleotide is a sequence used for priming a reaction by a polymerase. For example, one or more primer sequences with sequence complementarity to one or more RNAs of interest can be used to amplify the one or more RNAs of interest, thereby selectively enriching these RNAs. In particular embodiments, an oligonucleotide with sequence complementarity to the complementary strand of captured RNA (e.g., cDNA) can bind to the cDNA. For example, biotinylated oligonucleotides with sequence complementary to one or more cDNAs of interest binds to the cDNA and can be selected using biotinylation-streptavidin affinity using any of a variety of methods known to the field (e.g., streptavidin beads).
(viii-b-7) Clearing Step. The sample within the matrix substrate may be cleared of analytes that are not targets of interest. For example, the sample can be cleared of proteins (also called “deproteination”) by enzymatic proteolysis. The clearing step may be performed before or after covalent immobilization of any target analytes or derivatives thereof.

The clearing step can be performed after covalent immobilization of target molecules (e.g., RNA or DNA), derivatives of target molecules (e.g., cDNA or amplicons), or intermediate probes (e.g., padlock probes or adapters) to a synthetic matrix substrate. Performing the clearing step after immobilization can enable any subsequent nucleic acid hybridization reactions to be performed under conditions where the sample has been substantially deproteinated, as by enzymatic proteolysis (“protein clearing”). This method can have the benefit of removing ribosomes and other RNA- or nucleic-acid-target-binding proteins from the target analytes (while maintaining spatial location), where the protein component may impede or inhibit capture probe binding, or may impede binding of sample analytes to the IC spatial array captured ligand assay.

The clearing step can include removing non-targets from the matrix substrate. The clearing step can include degrading the non-targets. The clearing step can include exposing the sample to an enzyme (e.g., a protease) able to degrade a protein. The clearing step can include exposing the sample to a detergent.

Proteins may be cleared from the sample using enzymes, denaturants, chelating agents, chemical agents, and the like, which may break down the proteins into smaller components and/or amino acids. These smaller components may be easier to remove physically, and/or may be sufficiently small or inert such that they do not significantly affect the background. Similarly, lipids may be cleared from the sample using surfactants or the like. One or more of these agents can be used, e.g., simultaneously or sequentially. Examples of suitable enzymes include proteinases such as proteinase K, proteases or peptidases, or digestive enzymes such as trypsin, pepsin, or chymotrypsin. Examples of suitable denaturants include guanidine HCl, acetone, acetic acid, urea, or lithium perchlorate. Examples of chemical agents able to denature proteins include solvents such as phenol, chloroform, guanidinium isocyananate, urea, formamide, etc. Examples of surfactants include Triton X-100 (polyethylene glycol p-(1,1,3,3-tetramethylbutyl)-phenyl ether), SDS (sodium dodecyl sulfate), Igepal CA-630, or poloxamers. Examples of chelating agents include ethylenediaminetetraacetic acid (EDTA), citrate, or polyaspartic acid. In particular embodiments, compounds such as these may be applied to the sample to clear proteins, lipids, and/or other components. For instance, a buffer solution (e.g., containing Tris or tris(hydroxymethyl) aminomethane) may be applied to the sample, then removed.

In particular embodiments, nucleic acids that are not target analytes may be cleared. These non-target nucleic acids may can be removed with an enzyme to degrade nucleic acid molecules. Examples of DNA enzymes that may be used to remove DNA include DNase I, dsDNase, a variety of restriction enzymes, etc. Examples of techniques to clear RNA include RNA enzymes such as RNase A, RNase T, or RNase H, or chemical agents, e.g., via alkaline hydrolysis (for example, by increasing the pH to greater than 10). Examples of systems to remove sugars or extracellular matrix include enzymes such as chitinase, heparinases, or other glycosylases. Examples of systems to remove lipids include enzymes such as lipidases, chemical agents such as alcohols (e.g., methanol or ethanol), or detergents such as Triton X-100 or sodium dodecyl sulfate. In this way, the background of the sample may be removed, which may facilitate analysis of the analytes.
(viii-b-8) Other Preparation Steps. Additional reagents can be added to a biological sample to perform various functions prior to analysis of the biological sample. Nuclease inhibitors such as DNase and RNase inactivating agents or protease inhibitors, and/or chelating agents such as EDTA, can be added to the biological sample. Furthermore, nucleases, such as DNase or RNAse, or proteases, such as pepsin or proteinase K, can be added to the sample. Additional reagents may be dissolved in a solution or applied as a medium to the sample. Additional reagents (e.g., pepsin) may be dissolved in HCl prior to applying to the sample. For example, hematoxylin, from an H&E stain, can be optionally removed from the biological sample by washing in dilute HCl (0.001M to 0.1M) prior to further processing. Pepsin can be dissolved in dilute HCl (0.001M to 0.1M) prior to further processing. Samples can be washed additional times (e.g., 2, 3, 4, 5, or more times) in dilute HCl prior to incubation with a protease (e.g., pepsin), but after proteinase K treatment.

The sample can be treated with one or more enzymes. For example, one or more endonucleases to fragment DNA, DNA polymerase enzymes, and dNTPs used to amplify nucleic acids can be added. Other enzymes that can also be added to the sample include polymerase, transposase, ligase, DNAse, and RNAse.

Reverse transcriptase enzymes can be added to the sample, including enzymes with terminal transferase activity, primers, and template switch oligonucleotides (TSOs). Template switching can be used to increase the length of a cDNA, e.g., by appending a predefined nucleic acid sequence to the cDNA. In particular embodiments, the appended nucleic acid sequence includes one or more ribonucleotides.

Additional reagents can be added to improve the recovery of one or more target molecules (e.g., cDNA molecules, mRNA transcripts). For example, addition of carrier RNA to an RNA sample workflow process can increase the yield of extracted RNA/DNA hybrids from the biological sample. Carrier molecules are useful when the concentration of input or target molecules is low as compared to remaining molecules.

Generally, single target molecules cannot form a precipitate, and addition of the carrier molecules can help in forming a precipitate. Some target molecule recovery protocols use carrier RNA to prevent small amounts of target nucleic acids present in the sample from being irretrievably bound. In particular embodiments, carrier RNA can be added immediately prior to a second strand synthesis step. In particular embodiments, carrier RNA can be added immediately prior to a second strand cDNA synthesis on oligonucleotides released from an array. In particular embodiments, carrier RNA can be added immediately prior to a post in vitro transcription cleanup step. In particular embodiments, carrier RNA can be added prior to amplified RNA purification and quantification. In particular embodiments, carrier RNA can be added before RNA quantification. In particular embodiments, carrier RNA can be added immediately prior to both a second strand cDNA synthesis and a post in vitro transcription clean-up step.

In particular embodiments, a sample can undergo isometric expansion as described in section (vi) Isometric Expansion.

In particular embodiments, analytes in a sample can be processed prior to interaction with a capture probe. For example, prior to interaction with capture probes, polymerization reactions catalyzed by a polymerase (e.g., DNA polymerase or reverse transcriptase) are performed in the sample. In particular embodiments, a primer for the polymerization reaction includes a functional group that enhances hybridization with the capture sequence.

Analytes are the molecules within a sample that include the sequence that will bind or hybridize with the capture sequence of the capture probe. The analyte is the target molecule in which the IC spatial array is designed to capture. For example, if the analyte is mRNA, the capture sequence can include a polyT sequence which will capture the polyA tail of mRNA.

In particular embodiments, analytes are preprocessed for library generation via next generation sequencing. For example, analytes can be processed by addition of a modification (e.g., ligation of sequences that allow interaction with capture probes). In particular embodiments, analytes (e.g., DNA or RNA) are fragmented using fragmentation techniques (e.g., using transposases and/or fragmentation buffers).

Fragmentation can be followed by a modification of the analyte. For example, a modification can be the addition through ligation of an adapter sequence that allows hybridization with the capture probe. In particular embodiments, where the analyte of interest is RNA, poly(A) tailing is performed. Addition of a poly(A) tail to RNA that does not contain a poly(A) tail can facilitate hybridization with a capture probe that includes a capture domain with a functional amount of poly(dT) sequence.

In particular embodiments, prior to interaction with capture probes, target-specific reactions are performed in the sample. Examples of target specific reactions include ligation of target specific adaptors, probes and/or other oligonucleotides, target specific amplification using primers specific to one or more analytes, and target-specific detection using in situ hybridization, DNA microscopy, and/or antibody detection. In particular embodiments, a capture probe includes capture sequences targeted to target-specific products (e.g., amplification or ligation).

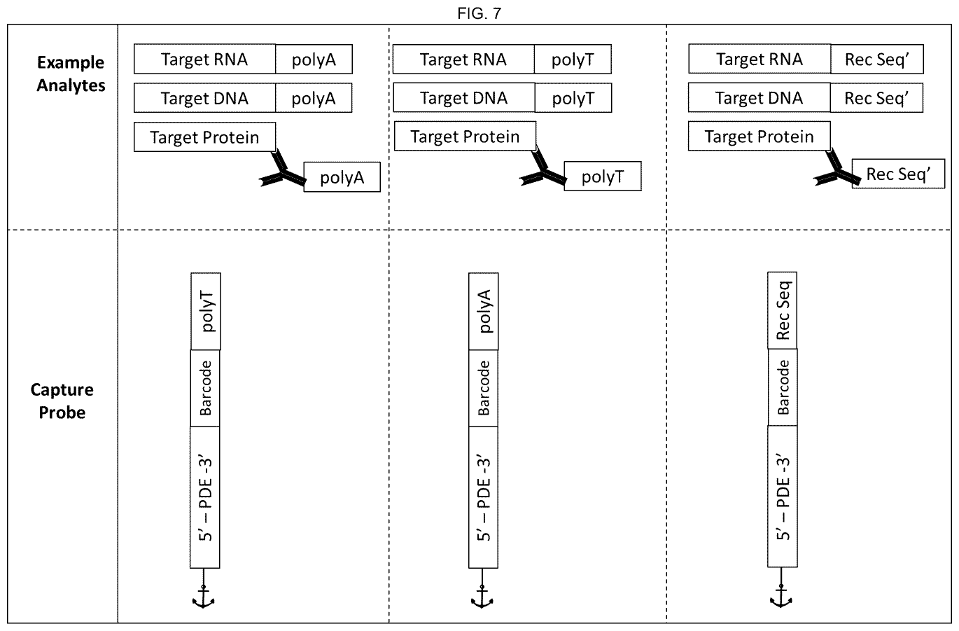

FIG. 7 shows examples of capture probes and the example analytes that could be captured by said capture probe. The target sequences and capture sequences include polyA sequences, polyT sequences, recognition sequences (Rec Seq), and complements to the recognition sequence (Rec Seq′).
(viii-c) Sample Transfer and Analyte Capture. In some examples of any of the methods described herein, an analyte in a sample can be transported (e.g., passively or actively) to a capture probe (e.g, a capture probe affixed to the IC spatial array).

A method to associate one or more analytes with barcoded capture probes is to promote analytes out of a sample and towards the spatially-barcoded array. The spatially-barcoded array populated with capture probes is situated with a sample. The sample can be permeabilized and a force can be generated to allow the target analyte to migrate away from the sample and toward the array. The target analyte interacts with a capture probe on the spatially-barcoded array.

As used herein, the term situated refers to the placement and position of the spatially barcoded array in relation to the sample such that analytes can migrate from the sample to the spatially barcoded array. In particular embodiments, the spatially barcoded array is situated in contact with the sample. In particular embodiments, the spatially barcoded array is situated in contact with a depletion layer which is situated in contact with the sample. In particular embodiments, the spatially barcoded array is situated such that it is substantially parallel to the sample. In particular embodiments, the spatially barcoded array is situated such that a buffer layer is disposed between the spatially barcoded array and the sample.

Examples of passive migration include simple diffusion and osmotic pressure created by the rehydration of dehydrated objects. Passive migration by diffusion uses concentration gradients. Diffusion is movement of untethered objects toward equilibrium. Therefore, when there is a region of high object concentration and a region of low object concentration, the object moves to an area of lower concentration. In some embodiments, untethered analytes move down a concentration gradient.

For example, analytes in a sample can be transported to a capture probe (e.g, an immobilized capture probe) using an electric field (e.g, using electrophoresis), a pressure gradient, fluid flow, gravity, a chemical concentration gradient, a temperature gradient, and/or a magnetic field. For example, analytes can be transported through, e.g, a gel (e.g, hydrogel matrix substrate), a fluid, a tissue, or a permeabilized cell, to a capture probe (e.g, an immobilized capture probe). In particular embodiments, an analyte can be transported through a depletion layer.

In particular embodiments, an electrophoretic field can be applied to analytes to facilitate migration of the analytes towards a capture probe. In particular embodiments, a sample contacts an IC spatial array, and an electric current is applied to promote the directional migration of charged analytes towards the capture probes fixed to the IC spatial array. An electrophoresis assembly, where a sample is in contact with a cathode and capture probes, and where the capture probes are in contact with the sample and an anode, can be used to apply the current.

In particular embodiments, electrophoretic transport and binding process is described by the Damkohler number (Da), which is a ratio of reaction and mass transport rates. The fraction of analytes bound and the shape of the sample will depend on the parameters in the Da. There parameters include electromigration velocity μₑ (depending on analyte electrophoretic mobility μe and electric field strength E), density of capture probes p₀, the binding rate between probes and analytes kₒₙ, and capture area thickness L.

$$Da ∼ \frac{{k}_{o n} {p}_{0} L}{{μ}_{e} E}$$

Fast migration (e.g., electromigration) can reduce assay time and can minimize molecular diffusion of analytes.

In particular embodiments, electrophoretic transfer of analytes can be performed while retaining the relative spatial alignment of the analytes in the sample. As such, an analyte captured by the capture probes linked to an IC spatial array retains the spatial information of the sample from which it was obtained. Applying an electrophoretic field to analytes can also result in an increase in temperature (e.g., heat). In particular embodiments, the increased temperature (e.g., heat) can facilitate the migration of the analytes towards a capture probe.

Migration refers to movement of an analyte from a cell that is overlaid onto a spatial array, to a capture probe that is attached to the surface of the array. Analytes released from cells may migrate in such a way that they contact capture probes within an array.

In particular embodiments, a spatially-addressable microelectrode array is used for spatially-constrained capture of at least one charged analyte of interest by a capture probe. For example, a spatially-addressable microelectrode array can allow for discrete (e.g., localized) application of an electric field rather than a uniform electric field. The spatially-addressable microelectrode array can be independently addressable. In particular embodiments, the electric field can be applied to one or more regions of interest in a biological sample. The electrodes may be adjacent to each other or distant from each other. The microelectrode array can be configured to include a high density of discrete sites having a small area for applying an electric field to promote the migration of charged analyte(s) of interest. For example, electrophoretic capture can be performed on a region of interest using a spatially-addressable microelectrode array.

A high density of discrete sites on a microelectrode array can be used. The surface can include any suitable density of discrete sites (e.g., a density suitable for processing the sample on the conductive substrate in a given amount of time). In one embodiment, the surface has a density of discrete sites greater than or equal to 500 sites per 1 mm². In particular embodiments, the surface has a density of discrete sites of 100, 200, 300, 400, 500, 600, 700, 800, 900, 1,000, 2,000, 3,000, 4,000, 5,000, 6,000, 7,000, 8,000, 9,000, 10,000, 20,000, 40,000, 60,000, 80,000, 100,000, or 500,000 sites per 1 mm². In particular embodiments, the surface has a density of discrete sites of at least 200, at least 300, at least 400, at least 500, at least 600, at least 700, at least 800, at least 900, at least 1,000, at least 2,000, at least 3,000, at least 4,000, at least 5,000, at least 6,000, at least 7,000, at least 8,000, at least 9,000, at least 10,000, at least 20,000, at least 40,000, at least 60,000, at least 80,000, at least 100,000, or at least 500,000 sites per 1 mm².

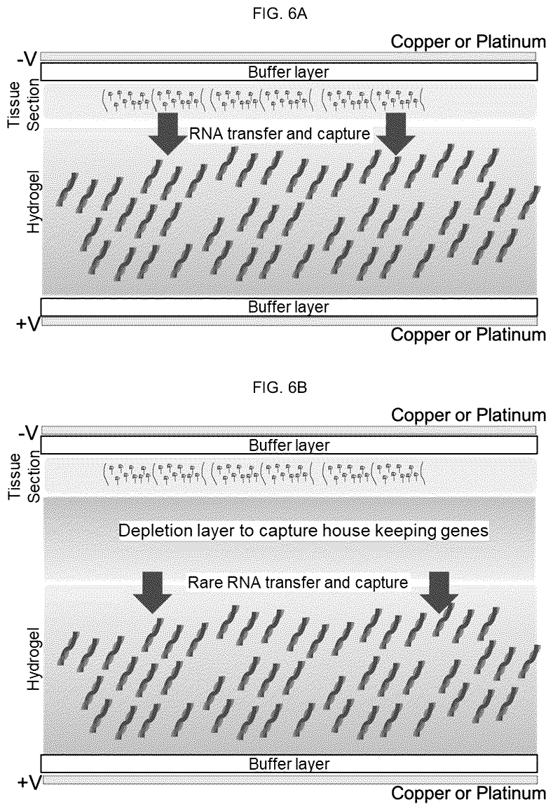

In an exemplary configuration (FIG. 6A) of an electrophoretic system, a sample is sandwiched between the cathode and the IC spatial array, and the IC spatial array is sandwiched between the sample (e.g., tissue section) and the anode, such that the sample is in contact with the IC spatial array. When an electric field is applied to the electrophoretic transfer system, negatively charged analytes (e.g., mRNA) will be pulled toward the positively charged anode and into the IC spatial array containing the spatially-barcoded capture probes. The spatially-barcoded capture probes interact with the analytes (e.g., mRNA transcripts hybridize to spatially-barcoded nucleic acid capture probes forming DNA/RNA hybrids), making the analyte capture more efficient. The electrophoretic system set-up may change depending on the target analyte. For example, proteins may be positive, negative, neutral, or polar depending on the protein as well as other factors (e.g., isoelectric point, solubility, etc.). The skilled practitioner has the knowledge and experience to arrange the electrophoretic transfer system to facilitate capture of a particular target analyte.

In another exemplary configuration (FIG. 6B) of an electrophoretic system, the anode and cathode sandwich a sample, depletion layer, and IC spatial array such that a cathode is in contact with a sample which is in contact with a depletion layer which is in contact with an IC spatial array which is in contact with an anode. When an electric field is applied to the electrophoretic transfer system, negatively charged analytes (e.g., mRNA) will be pulled toward the positively charged anode and into the depletion layer where house-keeping genes will be captured. Rare analytes will continue to travel toward the anode into the IC spatial array containing the spatially-barcoded capture probes. The spatially-barcoded capture probes interact with the analytes (e.g., mRNA transcripts hybridize to spatially-barcoded nucleic acid capture probes forming DNA/RNA hybrids), making the analyte capture more efficient.

In particular embodiments, the anode and cathode sandwich a sample and IC spatial array such that the cathode is in contact with the sample which is in contact with the IC spatial array which is in contact with the anode. In particular embodiments, the anode and cathode sandwich a sample and IC spatial array such that the anode is in contact with the sample which is in contact with the IC spatial array which is in contact with the cathode.

In particular embodiments, the anode and cathode sandwich a sample, depletion layer, and IC spatial array such that the cathode is in contact with the sample which is in contact with the depletion layer which is in contact with the IC spatial array which is in contact with the anode. In particular embodiments, the anode and cathode sandwich a sample, depletion layer, and IC spatial array such that the anode is in contact with the sample which is in contact with the depletion layer which is in contact with the IC spatial array which is in contact with the cathode.

In particular embodiments a buffer layer exists between the anode and cathode and either the sample and/or IC spatial array.

Once the spatially-barcoded capture probe is associated with a particular analyte, the sample can be optionally removed for analysis. The sample can be optionally dissociated before analysis. Once the tagged analyte is associated with the spatially-barcoded capture probe, the capture probes can be analyzed to obtain spatially-resolved information about the tagged analyte.
(viii-d) Depletion Layer. In particular embodiments, the analytes optionally travel through a depletion layer before being captured in the IC spatial array. In particular embodiments, the depletion layer captures abundant and house-keeping genes. In particular embodiments, the depletion layer allows rare analytes to pass through. Rare analytes are any analytes that are not considered house-keeping analytes. Analytes considered to be house-keeping analytes will depend on the experiment. House-keeping analytes are genes that are typically constitutive genes that are required for the maintenance of basic cellular function and are expressed in all cells of an organism under normal conditions. House-keeping analytes include proteins or DNA or RNA encoding the proteins. Examples of house-keeping analytes include GAPDH, beta-actin (ACTB), TAT-binding protein (TBP), ribosomal proteins (RP), ribosomal RNA, albumins, and tubulins.

The depletion layer can include a physical area in which clearing steps are performed. For example, the depletion layer can include enzymes, chemical agents, or detergents. The depletion layer can include antibodies. The depletion layer can include hybridizing sequences. In particular embodiments, one or more species of analyte (e.g., ribosomal and/or mitochondrial RNA) can be depleted using any of a variety of methods. Depleted means down-selected or removed. Examples of a hybridization and capture method of ribosomal RNA depletion include RiboMinus™ (Invitrogen Corporation, Waltham, MA), RiboCop™ (Lexogen GMBH, Vienna, Austria), and Ribo-Zero™ (Illumina, Inc. San Diego, CA). Another RNA depletion method involves hybridization of complementary DNA oligonucleotides to unwanted RNA followed by degradation of the RNA/DNA hybrids using RNase H. Examples of a hybridization and degradation method include NEBNext® (New England Biolabs, Inc., Ipswich, MA) rRNA depletion, NuGEN AnyDeplete, or RiboZero™ (Illumina, Inc.).

Another ribosomal RNA depletion method includes ZapR digestion, for example SMARTer. In the SMARTer method, random nucleic acid adapters are hybridized to RNA for first-strand synthesis and tailing by reverse transcriptase, followed by template switching and extension by reverse transcriptase. Additionally, first round PCR amplification adds full-length Illumina sequencing adapters (e.g., Illumina indexes). Ribosomal RNA is cleaved by ZapR v2 and R probes v2. A second round of PCR is performed, amplifying non-rRNA molecules (e.g., cDNA). Parts or steps of these ribosomal depletion protocols/kits can be further combined with the methods described herein to optimize protocols for a specific sample.

The depletion layer can include molecules that selectively hybridize to ribosomal RNA (rRNA), thereby reducing the pool and concentration of rRNA in the sample. The depletion layer can include molecules that selectively hybridize to mitochondrial RNA (mtRNA), thereby reducing the pool and concentration of mtRNA in the sample. Additionally and alternatively, the depletion layer can include duplex-specific nuclease (DSN) which can remove rRNA (see, e.g., Archer, et al, Selective and flexible depletion of problematic sequences from RNA-seq libraries at the cDNA stage, BMC Genomics, 15 401, (2014)). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53 (6) 373-80, (2012)).
(viii-e) Analyte Product Analysis. After analytes from the sample have hybridized or otherwise been associated with capture probes, the barcoded constructs that result from hybridization/association can be reverse transcribed to form a product and then analyzed via sequencing to identify the analytes.

With the analytes associated with the capture probes, reverse transcription can be performed in order to create a molecule having the complementary sequence to the analyte and a complementary sequence to the barcode of the capture probe. The “product” of such a reaction includes a sequence including the sequence or complement thereof of the analyte and a sequence or complement thereof of the spatial barcode. The product sequence may also include other sequences included in the capture probe such as the PDE or other functional sequences.

In particular embodiments, the methods described herein can be used to assess analyte levels and/or expression in a sample over time (e.g., before or after treatment with an agent or different stages of differentiation). In particular embodiments, the methods described herein can be performed on multiple similar samples obtained from the subject at a different time points (e.g., before or after treatment with an agent, different stages of differentiation, different stages of disease progression, different ages of the subject, or before or after development of resistance to an agent).

In particular embodiments, the product includes cDNA. Reverse transcription can be performed by RT-PCR. Using a reverse transcriptase enzyme and purified RNA template, one strand of cDNA is produced (referred to as first-strand cDNA synthesis). A first strand cDNA reaction can be optionally performed using template switching oligonucleotides. For example, a template switching oligonucleotide can hybridize to a poly(C) sequence added to a 3′ end of the cDNA by a reverse transcriptase enzyme. The original mRNA template and template switching oligonucleotide can then be denatured from the cDNA and the spatially-barcoded capture probe can then hybridize with the cDNA and a complement of the cDNA can be generated. The first strand cDNA can then be purified and collected for downstream amplification steps. The first strand cDNA can be optionally amplified using PCR, where the forward and reverse primers flank the spatial barcode and target analyte regions of interest, generating a library associated with a particular spatial barcode. In particular embodiments, the cDNA includes a sequencing by synthesis (SBS) primer sequence. In particular embodiments, reverse transcription (RT) reagents can be added to the sample. Incubation with the RT reagents can produce spatially-barcoded full-length cDNA from the captured analytes. Second strand reagents (e.g., second strand primers, enzymes) can be added to the sample on the IC spatial array to initiate second strand synthesis. The resulting cDNA can be denatured from the capture probe and transferred for amplification, sequencing, and/or library construction.

In particular embodiments, the capture probes can be optionally cleaved from the IC spatial array before product synthesis. In particular embodiments, the capture probes can be cleaved from the IC spatial array after product synthesis.

After product is synthesized (e.g., by reverse transcription) the product can be optionally amplified. Once a full-length product (e.g., cDNA molecule) is generated, the template switching oligonucleotide can serve as a primer in an amplification reaction (e.g., with a DNA polymerase). In particular embodiments, double stranded cDNA (e.g., first strand cDNA and second strand reverse complement cDNA) can be amplified via isothermal amplification with either a helicase or recombinase, followed by a strand displacing DNA polymerase. The strand displacing DNA polymerase can generate a displaced second strand resulting in an amplified product. Generating multiple copies of the product via amplification on the IC spatial array can improve final sequencing library complexity. Any method of amplification known to those skilled in the art can be used including PCR or isothermal nucleic acid amplification.

In particular embodiments, isothermal amplification can be linear amplification (e.g., asymmetrical with a single primer), or exponential amplification (e.g., with two primers). Isothermal nucleic acid amplification can be performed by a template-switching oligonucleotide primer which can add a common sequence onto the 5′ end of the nucleotide molecule being reverse transcribed. For example, after a capture probe interacts with an analyte (e.g., mRNA) and reverse transcription is performed such that additional nucleotides are added to the end of the cDNA creating a 3′ overhang as described herein. In particular embodiments, a template switching oligonucleotide hybridizes to untemplated poly(C) nucleotides added by a reverse transcriptase to continue replication to the 5′ end of the template switching oligonucleotide, thereby generating full-length cDNA ready for further amplification. In particular embodiments, the template switching oligonucleotide adds a common 5′ sequence to full-length cDNA that is used for cDNA amplification (e.g., a reverse complement of the template switching oligonucleotide).

After product is synthesized or if amplified, after amplification, the product is sequenced. A wide variety of different sequencing methods can be used to analyze the product (barcoded analyte constructs). In general, sequenced polynucleotides can be, for example, nucleic acid molecules such as deoxyribonucleic acid (DNA) or ribonucleic acid (RNA), including variants or derivatives thereof (e.g., single stranded DNA, cDNA, DNA/RNA hybrids, and nucleic acid molecules with a nucleotide analog).

Sequence analysis of the nucleic acid molecules (including barcoded nucleic acid molecules or derivatives thereof) can be direct or indirect. Thus, the sequence analysis substrate (which can be viewed as the molecule which is subjected to the sequence analysis step or process) can be the spatially barcoded nucleic acid molecule or it can be a molecule which is derived therefrom (e.g., a complement thereof). Thus, for example, in the sequence analysis step of a sequencing reaction, the sequencing template can be the spatially barcoded nucleic acid molecule or it can be a molecule derived therefrom. For example, a first and/or second strand DNA molecule can be subjected to sequence analysis (e.g., sequencing), i.e., can take part in the sequence analysis reaction or process (e.g., the sequencing reaction or sequencing process, or be the molecule which is sequenced or otherwise identified). Alternatively, the spatially barcoded nucleic acid molecule can be subjected to a step of second strand synthesis or amplification before sequence analysis (e.g., sequencing or identification by another technique). The sequence analysis substrate (e.g., template) can thus be an amplicon or a second strand of a spatially barcoded nucleic acid molecule.

In particular embodiments, both strands of a double stranded molecule can be subjected to sequence analysis (e.g., sequenced). In particular embodiments, single stranded molecules (e.g., spatially barcoded nucleic acid molecules) can be analyzed (e.g., sequenced). To perform single molecule sequencing, the nucleic acid strand can be modified at the 3′ end.

Sequencing of polynucleotides can be performed by various commercial systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification.

Other examples of methods for sequencing genetic material include DNA hybridization methods (e.g., Southern blotting), restriction enzyme digestion methods, Sanger sequencing methods, next-generation sequencing methods (e.g., single-molecule real time sequencing, nanopore sequencing, and Polony sequencing), ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by synthesis, real-time sequencing, reverse-terminator sequencing, nanopore sequencing, MS-PET sequencing, Laser Capture Microdissection couple with PolyA-based mRNA sequencing (LCM-Seq), and any combinations thereof.

Massively parallel pyrosequencing techniques can be used for sequencing nucleic acids. In pyrosequencing, the nucleic acid is amplified inside water droplets in an oil solution (emulsion PCR), with each droplet containing a single nucleic acid template attached to a single primer-coated bead that then forms a clonal colony. The sequencing system contains many picoliter-volume wells each containing a single bead and sequencing enzymes. Pyrosequencing uses luciferase to generate light for detection of the individual nucleotides added to the nascent nucleic acid and the combined data are used to generate sequence reads.

As another example application of pyrosequencing, released pyrophosphate (PPi) can be detected by being immediately converted to adenosine triphosphate (ATP) by ATP sulfurylase, and the level of ATP generated can be detected via luciferase-produced photons, such as described in Ronaghi, et al., Anal. Biochem. 242 (1), 84-9 (1996); Ronaghi, Genome Res. 11 (1), 3-11 (2001); Ronaghi et al. Science 281 (5375), 363 (1998); and U.S. Pat. Nos. 6,210,891, 6,258,568, and 6,274,320.

In particular embodiments, a massively parallel sequencing technique can be based on reversible dye-terminators. Four types of ddNTPs are added, and non-incorporated nucleotides are washed away. Unlike pyrosequencing, the DNA is only extended one nucleotide at a time due to a blocking group (e.g., 3′ blocking group present on the sugar moiety of the ddNTP). A detector acquires images of the fluorescently labelled nucleotides, and then the dye along with the terminal 3′ blocking group is chemically removed from the DNA, as a precursor to a subsequent cycle. This process can be repeated until the required sequence data is obtained.

In particular embodiments, sequencing is performed by detection of hydrogen ions that are released during the polymerization of DNA. A microwell containing a template DNA strand to be sequenced can be flooded with a single type of nucleotide. If the introduced nucleotide is sufficiently complementary to the leading template nucleotide, it is incorporated into the growing complementary strand. This causes the release of a hydrogen ion that triggers a hypersensitive ion sensor, which indicates that a reaction has occurred. If homopolymer repeats are present in the template sequence, multiple nucleotides will be incorporated in a single cycle. This leads to a corresponding number of released hydrogen ions and a proportionally higher electronic signal.

For analytes that have been barcoded via partitioning, barcoded nucleic acid molecules or derivatives thereof (e.g., barcoded nucleic acid molecules to which one or more functional sequences have been added, or from which one or more domains have been removed) can be pooled and processed together for subsequent analysis such as sequencing on high throughput sequencers. Processing with pooling can be implemented using barcode sequences. For example, barcoded nucleic acid molecules of a given partition can have the same barcode, which is different from barcodes of other spatial partitions. Alternatively, barcoded nucleic acid molecules of different partitions can be processed separately for subsequent analysis (e.g., sequencing).

In particular embodiments, analyte analysis uses direct sequencing of one or more captured analytes, such as direct sequencing of hybridized RNA. In particular embodiments, direct sequencing is performed after reverse transcription of hybridized RNA. In particular embodiments direct sequencing is performed after amplification of reverse transcription of hybridized RNA.

In particular embodiments, direct sequencing of captured RNA is performed by sequencing-by-synthesis (SBS). In particular embodiments, a sequencing primer is sufficiently complementary to a sequence in one or more of the domains of a capture probe. In such embodiments, sequencing-by-synthesis can include reverse transcription and/or amplification in order to generate a template sequence from which a primer sequence can bind.

SBS can involve hybridizing an appropriate primer, sometimes referred to as a sequencing primer, with the nucleic acid template to be sequenced, extending the primer, and detecting the nucleotides used to extend the primer. The nucleic acid used to extend the primer is detected before a further nucleotide is added to the growing nucleic acid chain, thus allowing base-by-base in situ nucleic acid sequencing. The detection of incorporated nucleotides is facilitated by including one or more labelled nucleotides in the primer extension reaction. To allow the hybridization of an appropriate sequencing primer to the nucleic acid template to be sequenced, the nucleic acid template should normally be in a single stranded form. If the nucleic acid templates making up the features are present in a double stranded form these can be processed to provide single stranded nucleic acid templates using methods well known in the art, for example by denaturation, cleavage, etc. The sequencing primers which are hybridized to the nucleic acid template and used for primer extension can be short oligonucleotides, for example, 15 to 25 nucleotides in length. The sequencing primers can be provided in solution or in an immobilized form. Once the sequencing primer has been annealed to the nucleic acid template to be sequenced by subjecting the nucleic acid template and sequencing primer to appropriate conditions, primer extension is carried out, for example using a nucleic acid polymerase and a supply of nucleotides, at least some of which are provided in a labelled form, and conditions suitable for primer extension if a suitable nucleotide is provided.

After each primer extension step, a washing step is included in order to remove unincorporated nucleotides which can interfere with subsequent steps. Once the primer extension step has been carried out, the nucleic acid colony is monitored to determine whether a labelled nucleotide has been incorporated into an extended primer. The primer extension step can then be repeated to determine the next and subsequent nucleotides incorporated into an extended primer. If the sequence being determined is unknown, the nucleotides applied to a given colony are usually applied in a chosen order which is then repeated throughout the analysis, for example dATP, dTTP, dCTP, dGTP.

SBS techniques which can be used are described for example in U.S. Patent App. Pub. No. 2007/0166705, U.S. Patent App. Pub. No. 2006/0188901, U.S. Pat. No. 7,057,026, U.S. Patent App. Pub. No. 2006/0240439, U.S. Patent App. Pub. No. 2006/0281109, PCT Patent App. Pub. No. WO 05/065814, U.S. Patent App. Pub. No. 2005/0100900, PCT Patent App. Pub. No. WO 06/064199, PCT Patent App. Pub. No. W007/010,251, U.S. Patent App. Pub. No. 2012/0270305, U.S. Patent App. Pub. No. 2013/0260372, and U.S. Patent App. Pub. No. 2013/0079232.

Sequential fluorescence hybridization can involve sequential hybridization of probes including degenerate primer sequences and a detectable label. A degenerate primer sequence is a short oligonucleotide sequence which is capable of hybridizing to any nucleic acid fragment independent of the sequence of said nucleic acid fragment. For example, such a method could include the steps of: (a) providing a mixture including four probes, each of which includes either A, C, G, or T at the 5′-terminus, further including degenerate nucleotide sequence of 5 to 11 nucleotides in length, and further including a functional domain (e.g., fluorescent molecule) that is distinct for probes with A, C, G, or T at the 5′-terminus; (b) associating the probes of step (a) to the target polynucleotide sequences, whose sequence needs will be determined by this method; (c) measuring the activities of the four functional domains and recording the relative spatial location of the activities; (d) removing the reagents from steps (a)-(b) from the target polynucleotide sequences; and repeating steps (a)-(d) for n cycles, until the nucleotide sequence of the spatial domain for each bead is determined, with modification that the oligonucleotides used in step (a) are complementary to part of the target polynucleotide sequences and the positions 1 through n flanking the part of the sequences. In particular embodiments, these additional flanking sequences are degenerate sequences. The fluorescent signal from each spot on the array for cycles 1 through n can be used to determine the sequence of the target polynucleotide sequences.

In particular embodiments, captured nucleic acid sequence is amplified prior to hybridization with a sequencing probe (e.g., reverse transcription to cDNA and amplification of cDNA). In particular embodiments, a capture probe containing captured nucleic acid sequence (i.e., analyte) is exposed to the sequencing probe targeting coding regions of nucleic acid sequence. In particular embodiments, one or more sequencing probes are targeted to each coding region. In particular embodiments, the sequencing probe is designed to hybridize with sequencing reagents (e.g., a dye-labeled readout oligonucleotides). A sequencing probe can then hybridize with sequencing reagents. In particular embodiments, output from the sequencing reaction is imaged. In particular embodiments, a specific sequence of cDNA is resolved from an image of a sequencing reaction. In particular embodiments, reverse transcription of captured nucleic acid sequence is performed prior to hybridization to the sequencing probe. In particular embodiments, the sequencing probe is designed to target complementary sequences of the coding regions of a nucleic acid sequence (e.g., targeting cDNA).

In particular embodiments, the nucleic acid sequence is RNA. In particular embodiments, a captured RNA is sequenced using a nanopore-based method. In particular embodiments, direct sequencing is performed using nanopore direct RNA sequencing in which captured RNA is translocated through a nanopore. A nanopore current can be recorded and converted into a base sequence. In particular embodiments, captured RNA remains attached to a substrate during nanopore sequencing. In particular embodiments, captured RNA is released from the substrate prior to nanopore sequencing. In particular embodiments, where the analyte of interest is a protein, direct sequencing of the protein can be performed using nanopore-based methods. Examples of nanopore-based sequencing methods that can be used are described in Deamer et al., Trends Biotechnol. 18, 14 7-151 (2000); Deamer et al, Acc. Chem. Res. 35:817-825 (2002); Li et al., Nat. Mater. 2:611-615 (2003); Soni et al., Clin. Chem. 53, 1996-2001 (2007); Healy et al., Nanomed. 2, 459-481 (2007); Cockroft et al., J. Am. Chem. Soc. 130, 818-820 (2008); and in U.S. Pat. No. 7,001,792.

In particular embodiments, direct sequencing of captured RNA is performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and attributes involved in sequencing by ligation are described, for example, in Shendure et al. Science (2005), 309:1728-1732, and in U.S. Pat. Nos. 5,599,675; 5,750,341; 6,969,488; 6,172,218; and 6,306,597.

In particular embodiments, nucleic acid hybridization can be used for sequencing. These methods utilize labeled nucleic acid decoder probes that are complementary to at least a portion of a spatial barcode sequence. Multiplex decoding can be performed with pools of many different probes with distinguishable labels. Examples of nucleic acid hybridization sequencing are described for example in U.S. Pat. No. 8,460,865, and in Gunderson et al., Genome Research 14:870-877 (2004).

In particular embodiments, commercial high-throughput digital sequencing techniques can be used to analyze spatial barcode sequences, in which DNA templates are prepared for sequencing not one at a time, but in a bulk process, and where many sequences are read out in parallel, or alternatively using an ultra-high throughput serial process that itself may be parallelized.

Examples of such techniques include Illumina® (San Diego, CA) sequencing (e.g., flow cell-based sequencing by synthesis techniques), using modified nucleotides (such as commercialized in HiSeq™ and additional sequencing technology instruments by Illumina, Inc., San Diego, CA), HeliScope™ by Helicos Biosciences Corporation, Cambridge, MA, and PacBio RS by Pacific Biosciences of California, Inc., Menlo Park, CA), sequencing by ion detection technologies (lon Torrent, Inc., South San Francisco, CA), and sequencing of DNA nanoballs (Complete Genomics, Inc., Mountain View, CA).

In particular embodiments, the product (e.g., cDNA) can be enzymatically fragmented and size-selected in order to optimize the product amplicon size. P5, P7, i7, and i5 can be used as sample indexes, and TruSeq Read 2 can be added via End Repair, A-tailing, Adaptor Ligation, and PCR. The product fragments can then be sequenced using paired-end sequencing using TruSeq Read 1 and TruSeq Read 2 as sequencing primer sites. See, Illumina, Indexed Sequencing Overview Guides, February 2018, Document 15057455v04; and Alumina Adapter Sequences, May 2019, Document #1000000002694vl 1, for information on P5, P7, i7, i5, TruSeq Read 2, indexed sequencing, and other reagents.

In particular embodiments, detection of a proton released upon incorporation of a nucleotide into an extension product can be used in the methods described herein. For example, the sequencing methods and systems described in U.S. Patent Application Publication Nos. 2009/0026082, 2009/0127589, 2010/0137143, and 2010/0282617, can be used to sequence spatial barcodes.

In particular embodiments, real-time monitoring of DNA polymerase activity can be used during sequencing. For example, nucleotide incorporations can be detected through fluorescence resonance energy transfer (FRET), as described for example in Levene et al, Science (2003), 299, 682-686, Lundquist et al., Opt. Lett. (2008), 33, 1026-1028, and Korlach et al., Proc. Natl. Acad. Sci. USA (2008), 105, 1176-1181.

Isothermal nucleic acid amplification can be used in addition to, or as an alternative to standard PCR reactions. Isothermal nucleic acid amplification generally does not require the use of a thermocycler, however in particular embodiments, isothermal amplification can be performed in a thermocycler. In particular embodiments, isothermal amplification can be performed from 35° C. to 75° C. In particular embodiments, isothermal amplification can be performed from 40° C., 45° C., 50° C., 55° C., 60° C., 65° C., or 70° C. or anywhere in between depending on the polymerase and auxiliary enzymes used.

Isothermal nucleic acid amplification techniques are known in the art, and can be used alone or in combination with any of the spatial methods described herein. For example, suitable isothermal nucleic acid amplification techniques include transcription mediated amplification, nucleic acid sequence-based amplification, signal mediated amplification of RNA technology, strand displacement amplification, rolling circle amplification, loop-mediated isothermal amplification of DNA (LAMP), isothermal multiple displacement amplification, recombinase polymerase amplification, helicase-dependent amplification, single primer isothermal amplification, and circular helicase-dependent amplification (See, e.g., Gill and Ghaemi, Nucleic acid isothermal amplification technologies: a review, Nucleosides, Nucleotides, & Nucleic Acids, 27 (3), 224-43, doi: 10.1080/15257770701845204 (2008)).

In particular embodiments, the isothermal nucleic acid amplification is helicase-dependent nucleic acid amplification. Helicase-dependent isothermal nucleic acid amplification is described in Vincent et. ah, 2004, Helicase-dependent isothermal DNA amplification, EMBO Rep., 795-800 and U.S. Pat. No. 7,282,328. Further, helicase-dependent nucleic acid amplification is described in Andresen et. ak, 2009, Helicase-dependent amplification: use in OnChip amplification and potential for point-of-care diagnostics, Expert Rev Mol Diagn. 9, 645-650, doi: 10.1586/erm.09.46. In particular embodiments, the isothermal nucleic acid amplification is recombinase polymerase nucleic acid amplification. Recombinase polymerase nucleic acid amplification is described in Piepenburg etal., 2006, DNA Detection Using Recombinant Proteins, PLOS Biol. 4, 7 e204 and Li el. al., 2019, Review: a comprehensive summary of a decade development of the recombinase polymerase amplification, Analyst 144, 31-67, doi: 10.1039/C8AN01621F (2019.

Generally, isothermal amplification techniques use standard PCR reagents (e.g., buffer, dNTPs etc.) known in the art. Some isothermal amplification techniques can require additional reagents. For example, helicase dependent nucleic acid amplification uses a single-strand binding protein and an accessory protein. In another example, recombinase polymerase nucleic acid amplification uses recombinase (e.g., T4 UvsX), recombinase loading factor (e.g., TF UvsY), single strand binding protein (e.g., T4 gp32), crowding agent (e.g., PEG-35K), and ATP.

After isothermal nucleic acid amplification of the full-length cDNA described by any of the methods herein, the isothermally amplified cDNAs (e.g., single-stranded or double-stranded) can be recovered from the substrate, and optionally followed by amplification with typical cDNA PCR in microcentrifuge tubes.
(viii-f) Spatial Analysis Methods. Array-based spatial analysis methods involve the transfer of one or more analytes from a sample to an array of features, each of which is associated with a unique spatial barcode. Subsequent analysis of the transferred analytes includes determining the identity of the analytes and the spatial location of each analyte within the sample. The spatial location of each analyte within the sample is determined based on the feature (e.g., discrete volume) to which each analyte is bound in the array, and the capture spot's relative spatial location within the array according to the barcode.

Provided herein are methods and arrays for spatially detecting an analyte (e.g., detecting the location of an analyte) from a sample (e.g., present in a biological sample such as a tissue section) that include: (a) providing a sample including analytes of interest; (b) providing an IC spatial array including one or more discrete volumes each with capture probes including barcodes unique to the discrete volume; (c) transferring the analytes of interest from the sample to the IC spatial array, thereby allowing the barcoded capture probes to capture the analyte of interest; and (d) analyzing the captured analyte.

In particular embodiments, reagents for performing spatial analysis are added to the sample before, contemporaneously with, or after the IC spatial array is situated with the sample. In particular embodiments, the IC spatial array is in contact with the sample. In particular embodiments, the IC spatial array is in contact with a depletion layer which is in contact with the sample. In particular embodiments, the sample and/or analyte can be processed (e.g., permeabilized) before performing spatial analysis.

The analytes are migrated toward the IC spatial array using any number of techniques disclosed herein. For example, analyte migration can occur using a diffusion-resistant medium lid and passive migration. As another example, analyte migration can be active migration, using an electrophoretic transfer system, for example. Once the analytes are in close proximity to the spatially-barcoded capture probes, the capture probes can hybridize or otherwise bind a target analyte. In particular embodiments, analytes from the sample are transported to a capture probe within the IC spatial array using electrophoresis. In particular embodiments, electrophoresis includes placing a sample and IC spatial array between an anode and cathode; and applying an electric field such that the analytes migrate into the IC spatial array. In particular embodiments, the analytes can migrate through a depletion layer to remove housekeeping analytes. In particular embodiments, the analytes can migrate in the direction of the anode or in the direction of the cathode. In particular embodiments, when the analytes migrate into the IC spatial array the, analytes of interest will be captured by the capture sequence of the capture probes. In particular embodiments, the capture sequence includes a sequence complementary to a sequence on the analyte such that the sequences hybridize. After analyte capture, the sample can be optionally removed from the array.

In particular embodiments, the IC spatial array includes spatially-barcoded capture probes that are clustered at areas called discrete volumes within the matrix substrate. The spatially-barcoded capture probes can include a cleavage domain, one or more functional sequences, a barcode, a unique molecular identifier, and a capture sequence. In particular embodiments, the spatially-barcoded capture probe includes a PDE, a barcode, and a capture sequence. The spatially-barcoded capture probes can also include a 5′ end modification for reversible attachment to the matrix substrate.

After capture by the capture probes, the analytes are analyzed. In particular embodiments, analysis of the analytes includes reverse transcribing hybridized analytes to form a product and sequencing the product. The product is the sequence or complement thereof including the analyte sequence and a portion of the capture probe sequence such that the product sequence can identify both the analyte and the barcode. After product synthesis, the product is denatured from the capture probe and can undergo amplification, sequencing, and/or library construction. In particular embodiments, the product is amplified before it is sequenced. In particular embodiments, the product is amplified using isothermal nucleic acid amplification.

The product and/or amplified product can be sequenced and/or processed to form a product library. The product can be sequenced by any method known in the art and is described in greater detail in the (viii-e) Analyte Product Analysis section. In particular embodiments, the product can be sequenced within the IC spatial array or the product can be removed from the IC spatial array and then sequenced.

In particular embodiments, the library preparation can be quantified and/or subjected to quality control to verify the success of the library preparation steps. The library amplicons are sequenced and analyzed to decode spatial information, with an additional library quality control (QC) step.

In particular embodiments, the product (e.g., cDNA), amplified product, or product library is quantified. In particular embodiments, the quantification of RNA and/or DNA is carried out by real-time PCR (also known as quantitative PCR or qPCR), using techniques well known in the art, such as TAQMAN® (Roche Molecular Systems, Inc., Pleasanton, CA) or SYBR® (Molecular Probes, Inc., Eugene, OR), or on capillaries (“LightCycler® (Roche Diagnostics Gmbh, Germany) Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

Resulting product, amplified product, or product library can be analyzed. In particular embodiments, performing correlative analysis of data can yield over 95% correlation of genes expressed across two capture areas (e.g., 95% or greater, 96% or greater, 97% or greater, 98% or greater, or 99% or greater). In particular embodiments, using single cell RNA sequencing of nuclei, correlative analysis of the data can yield over 90% (e.g., over 90%, 91%, 92%, 93%, 94%, 95%, 96%, 97%, 98%, or 99%) correlation of genes expressed across two capture areas.

Aspects of spatial analysis methodologies are described in International Application Publications No. WO 2020/198071, WO 2019/075091, WO 2019/068880, WO 2018/136856, WO 2018/107054, WO 2018/091676, WO 2018/057999, WO 2018/045186, WO 2018/045181, WO 2018/022809, WO 2017/222453, WO 2017/147483, WO 2017/144338, WO 2017/027368, WO 2017/027367, WO 2016/166128, WO 2016/162309, WO 2016/057552, WO 2016/007839, WO 2015/161173, WO 2014/210233, WO 2014/210225, WO 2014/163886, WO 2012/140224, WO 2014/060483, WO 2011/127099, WO 2011/094669; U.S. Pat. Nos. 10,059,990, 10,041,949, 10,002,316, 9,783,841, 9,727,810, 8,951,726, 8,604, 182, and 7,709,198; U.S. Patent Application Publications No. 2018/0245142 and 2017/0016053; as well as in Rodriques et al. (Science 363 (6434): 1463-1467, 2019), Trejo et al. (PLOS ONE 14 (2): e0212031, 2019), Gupta et al. (Nature Biotechnol. 36:1 197-1202, 2018), Gao et al. (BMC Biol. 15:50, 2017), Chen et al. (Science 348 (6233): aaa6090, 2015), and Lee et al. (Nat. Protoc. 10 (3): 442-458, 2015). These methodologies can be used herein in any combination.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.
(ix) Exemplary Embodiments.
1. An increased capture potential (IC) spatial array including
   a matrix substrate, wherein the matrix substrate includes an array, a thickness, and a plurality of discrete volumes, wherein a discrete volume occupies a distinct x and y position on the array and continues into the thickness of the matrix substrate in a z direction, and wherein the discrete volume includes a plurality of capture probes with barcodes unique to the discrete volume anchored to the matrix substrate.
2. The IC spatial array of embodiment 1, wherein a majority of the plurality of capture probes have a free end.
3. The IC spatial array of embodiment 2, wherein 90% of the plurality of capture probes have a free end.
4. The IC spatial array of embodiments 2 or 3, wherein the plurality of capture probes each have a free end.
5. The IC spatial array of any of embodiments 1-4, wherein the discrete volume is 1 μm to 30 μm in the x and/or y direction.
6. The IC spatial array of any of embodiments 1-5, wherein the discrete volume is 1 μm to 3 cm in the z direction
7. The IC spatial array of any of embodiments 1-6, wherein a majority of capture probes include a barcode and a capture sequence.
8. The IC spatial array of embodiment 7, wherein 90% of the capture probes include a barcode and a capture sequence.
9. The IC spatial array of embodiments 7 or 8, wherein each of the capture probes include a barcode and a capture sequence.
10. The IC spatial array of embodiments 8 or 9, wherein the capture sequence binds to a target sequence on an analyte.
11. The IC spatial array of embodiment 10, wherein the analyte is RNA, DNA, or a protein.
12. The IC spatial array of embodiments 10 or 11, wherein the target sequence includes a polyA sequence and the capture sequence includes a polyT sequence.
13. The IC spatial array of embodiment 12, wherein the polyT sequence includes a 10-50 nucleotide polyT sequence.
14. The IC spatial array of embodiment 13, wherein the polyT sequence includes the sequence TTTTTTTT TTTTTTTTT (SEQ ID NO: 2).
15. The IC spatial array of embodiments 10 or 11, wherein the target sequence includes a polyT sequence and the capture sequence includes a polyA sequence.
16. The IC spatial array of embodiment 15, wherein the polyA sequence includes a 10-50 nucleotide polyA sequence.
17. The IC spatial array of embodiments 15 or 16, wherein the polyA sequence includes the sequence AAAAAAAAAAAAAAAAAAAA (SEQ ID NO: 1).
18. The IC spatial array of embodiments 10 or 11, wherein the target sequence includes a polyA sequence and the capture sequence includes a polyU sequence.
19. The IC spatial array of embodiment 18, wherein the polyU sequence includes a 10-50 nucleotide polyU sequence.
20. The IC spatial array of embodiments 18 or 19, wherein the polyU sequence includes the sequence UUUUUUUUUUUUUUUUUUUU (SEQ ID NO: 3).
21. The IC spatial array of any of embodiments 10-20, wherein the capture sequence includes a sequence that hybridizes to the target sequence.
22. The IC spatial array of any of embodiments 1-21, wherein the capture probe includes a functional sequence.
23. The IC spatial array of embodiment 22, wherein the functional sequence includes a cleavage domain, unique molecular identifier, or spacer.
24. The IC spatial array of any of embodiments 1-23, wherein the matrix substrate includes a hydrogel.
25. The IC spatial array of embodiment 24, wherein the hydrogel includes a polyacrylamide gel.
26. The IC spatial array of any of embodiments 1-25, wherein the matrix substrate includes a solid state membrane.
27. The IC spatial array of embodiment 26, wherein the solid state membrane includes glass.
28. The IC spatial array of embodiment 27, wherein the solid state membrane including glass is rigid and porous.
29. The IC spatial array of any of embodiments 1-28, wherein the matrix substrate is suitable for electrophoresis.
30. The IC spatial array of any of embodiments 1-29, wherein the matrix substrate is 10 μm-2 mm thick.
31. The IC spatial array of any of embodiments 1-30, wherein discrete volumes within the matrix substrate include at least 500,000 capture probes.
32. The IC spatial array of any of embodiments 1-31, wherein the plurality of capture probes are linked to the matrix substrate with 5′ acrydite.
33. The IC spatial array of any of embodiments 1-32, wherein discrete volumes within the matrix substrate are separated by a physical structure.
34. The IC spatial array of any of embodiments 1-33, wherein the IC spatial array includes a capillary array.
35. A starting matrix substrate including primer docking elements (PDEs) and capture sequence primers, wherein the PDEs are anchored to the matrix substrate.
36. The starting matrix substrate of embodiment 35, wherein the capture sequence primers are anchored to the matrix substrate.
37. The starting matrix substrate of embodiments 35 or 36, wherein the capture sequence primers are not anchored to the matrix substrate.
38. The starting matrix substrate of any of embodiments 35-37, wherein the capture sequence primers include a polyT sequence.
39. The starting matrix substrate of embodiment 38, wherein the polyT sequence includes a 10-50 nucleotide polyT sequence.
40. The starting matrix substrate of embodiment 39, wherein the polyT sequence includes

(SEQ ID NO: 2)
TTTTTTTTTTTTTTTTTTTT.

41. The starting matrix substrate of any of embodiments 35-37, wherein the capture sequence primers include a polyA sequence.
42. The starting matrix substrate of embodiment 41, wherein the polyA sequence includes a 10-50 nucleotide polyA sequence.
43. The starting matrix substrate of embodiment 42, wherein the polyA sequence includes

(SEQ ID NO: 1)
AAAAAAAAAAAAAAAAAAAA.

44. The starting matrix substrate of any of embodiments 35-37, wherein the capture sequence primers include a polyU sequence.
45. The starting matrix substrate of embodiment 44, wherein the polyU sequence includes a 10-50 nucleotide polyU sequence.
46. The starting matrix substrate of embodiment 45, wherein the polyU sequence includes

(SEQ ID NO: 3)
UUUUUUUUUUUUUUUUUUUU.

47. The starting matrix substrate of any of embodiments 35-46, wherein the capture sequence primers include a recognition sequence.
48. The starting matrix substrate of any of embodiments 35-47, wherein the PDEs are not anchored to the matrix substrate.
49. The starting matrix substrate of any of embodiments 35-48, wherein the matrix substrate includes a hydrogel.
50. The starting matrix substrate of any of embodiments 35-49, wherein the matrix substrate includes a polyacrylamide gel.
51. The starting matrix substrate of any of embodiments 35-50, wherein the PDEs are anchored to the matrix substrate with 5′ acrydite.
52. The starting matrix of any of embodiments 35-51, wherein the capture sequence primer is anchored to the matrix substrate with 5′ acrydite.
53. The starting matrix substrate of any of embodiments 35-52, wherein the matrix substrate is isometrically expanded.
54. The starting matrix substrate of any of embodiments 35-53, wherein the matrix substrate is 50 μm-500 μm thick.
55. The starting matrix of any of embodiments 35-54, wherein the starting matrix includes a capillary array.
56. A kit including a template array and a starting matrix substrate of any of embodiments 35-55.
57. The kit of embodiment 56, wherein the template array includes a substrate including an array with a plurality of discrete areas.
58. The kit of embodiment 57, wherein the discrete areas include a plurality of template probes unique to the discrete area.
59. The kit of embodiments 57 or 58, wherein the discrete areas occupy discrete locations on the array.
60. The kit of any of embodiments 57-59, wherein discrete areas within the array are separated by a physical structure.
61. The kit of any of embodiments 57-60, wherein discrete areas are separated by a physical structure.
62. The kit of any of embodiments 58-61, wherein a majority of template probes have a free end.
63. The kit of embodiment 62, wherein 90% of the template probes have a free end.
64. The kit of any of embodiments 58-63, wherein each template probe has a free end.
65. The kit of any of embodiments 58-64, wherein the template probe includes a PDE, a barcode template, and a capture sequence template.
66. The kit of embodiment 65, wherein the barcode template includes a nucleic acid sequence unique to the discrete area.
67. The kit of any of embodiments 58-66, wherein the template probe includes a nucleic acid sequence.
68. The kit of any of embodiments 65-67, wherein the capture sequence template binds to a capture sequence primer in the starting matrix substrate.
69. The kit of embodiment 68, wherein the capture sequence template includes a polyT sequence and the capture sequence primer includes a polyA sequence.
70. The kit of embodiment 68, wherein the capture sequence template includes a polyA sequence and the capture sequence primer includes a polyT sequence.
71. The kit of embodiment 68, wherein the capture sequence template includes a polyA sequence and the capture sequence primer includes a polyU sequence.
72. The kit of any of embodiments 69-71, wherein the polyA sequence includes a 10-50 nucleotide polyA sequence.
73. The kit of any of embodiments 69 or 70, wherein the polyT sequence includes a 10-50 nucleotide polyT sequence.
74. The kit of embodiment 71, wherein the polyU sequence includes a 10-50 nucleotide polyUsequence.
75. The kit of any of any of embodiments 69-74, wherein the polyA sequence includes the sequence AAAAAAAAAAAAAAAAAAAA (SEQ ID NO: 1).
76. The kit of any of embodiments 69-74, wherein the polyT sequence includes the sequence

(SEQ ID NO: 2)
TTTTTTTTTTTTTTTTTTTT.

77. The kit of any of embodiments 71-74, wherein the polyU sequence includes the sequence

(SEQ ID NO: 3)
UUUUUUUUUUUUUUUUUUUU.

78. The kit of any of embodiments 68-77, wherein the capture sequence template includes a recognition sequence and the capture sequence primer includes a sequence that hybridizes to the recognition sequence.
79. The kit of any of embodiments 68-78, wherein the capture sequence primer includes a sequence that is that hybridizes to the capture sequence template.
80. The kit of any of embodiments 58-79, wherein the template probe includes a functional sequence.
81. The kit of embodiment 80, wherein the functional sequence includes a cleavage domain, unique molecular identifier, or spacer.
82. The kit of any of embodiments 56-81, further including PCR-enabling media.
83. The kit of embodiment 82, wherein the PCR-enabling media includes a polymerase and extension reagents.
84. The kit of embodiment 83, wherein the polymerase includes Taq DNA polymerase.
85. The kit of embodiments 83 or 84, wherein the extension reagents include PCR master mix.
86. A method of producing an increased capture potential (IC) spatial array including:
   (a) placing a matrix substrate in contact with a template array in a PCR-enabling media, wherein the matrix substrate includes (1) final primer docking elements (PDEs) anchored to the matrix substrate and (2) capture sequence primers (CSPs), and wherein the template array includes (1) a plurality of discrete area, wherein the discrete area contains a plurality of template probes, wherein the template probes include a PDE template, a barcode template, and a capture sequence template;
   (b) adjusting the environment of the PCR-enabling media such that the CSPs hybridize with the capture sequence templates, the CSPs extend through the template probes to create bridge probes, wherein the bridge probe includes a sequence that hybridizes to the template probe and includes a CSP, bridge barcode, and a bridge PDE; the bridge probes dissociate from the template probes; the bridge PDEs hybridize with the PDE of the matrix substrate; the PDE extends through the bridge probes to create capture probes, wherein the capture probe includes a sequence that hybridizes to the bridge probe and includes a capture sequence, a barcode, and a PDE; and the capture probes dissociate from the bridge probes, wherein the capture sequence and capture sequence template have the same sequence and are complements to the CSP, the barcode and barcode template have the same sequence and are complements to the bridge barcode, and the PDE and PDE template are the same sequence and are complements to the bridge PDE.
87. The method of embodiment 86, wherein the matrix substrate includes a hydrogel.
88. The method of embodiments 86 or 87, wherein the matrix substrate includes a polyacrylamide gel.
89. The method of any of embodiments 86-88, further including isometrically expanding the matrix substrate, wherein isometrically expanding the matrix substrate includes anchoring one or more components of a sample to a gel, followed by gel formation, proteolysis, and swelling.
90. The method of any of embodiments 86-89, further including shrinking the matrix substrate.
91. The method of embodiment 90, wherein shrinking the matrix substrate includes exposing the matrix substrate to a dehydrating solvent, a salt, heat, a vacuum, lyophilization, desiccation, filtration, or air-drying.
92. The method of any of embodiments 86-91, further including isometrically expanding the matrix substrate and then shrinking the expanded matrix substrate.
93. The method of any of embodiments 86-92, wherein the matrix substrate is isometrically expanded before producing the IC spatial array and then shrunk after producing the IC spatial array.
94. The method of any of embodiments 86-93, wherein the matrix substrate is 50 μm-500 μm thick.
95. The method of any of embodiments 86-94, further including anchoring the CSPs to the matrix substrate.
96. The method of any of embodiments 86-94, wherein the CSPs are not anchored to the matrix substrate.
97. The method of any of embodiments 86-96, further including anchoring the PDEs to the matrix substrate using 5′ acrydite.
98. The method of any of embodiments 86-97, further including anchoring the CSPs to the matrix substrate using 5′ acrydite.
99. The method of any of embodiments 86-98, wherein the capture sequence template includes a polyT sequence.
100. The method of embodiment 99, wherein the polyT sequence includes a 10-50 nucleotide polyT sequence.
101. The method of embodiment 100, wherein the polyT sequence includes

(SEQ ID NO: 2)
TTTTTTTTTTTTTTTTTTTT.

102. The method of any of embodiments 86-98, wherein the capture sequence template includes a polyA sequence.
103. The method of embodiment 102, wherein the polyA sequence includes a 10-50 nucleotide polyA sequence.
104. The method of embodiment 103, wherein the polyA sequence includes

(SEQ ID NO: 1)
AAAAAAAAAAAAAAAAAAAA.

105. The method of any of embodiments 86-98, wherein the capture sequence template includes a polyU sequence.
106. The method of embodiment 105, wherein the polyU sequence includes a 10-50 nucleotide polyU sequence.
107. The method of embodiment 106, wherein the polyU sequence includes

(SEQ ID NO: 3)
UUUUUUUUUUUUUUUUUUUU.

108. The method of any of embodiments 86-107, wherein the capture sequence template includes a recognition sequence.
109. The method of any of embodiments 86-108, wherein the discrete area occupies a discrete x and y location on the template array.
110. The method of any of embodiments 86-109, wherein template probes have barcode templates that are unique to the discrete area in which the template probe is located.
111. The method of any of embodiments 86-110, wherein the barcode template is unique to the position of the template probe on the template array.
112. The method of any of embodiments 86-111, wherein the template probe has a free end.
113. The method of any of embodiments 86-112, wherein the PCR-enabling media includes extension reagents and a polymerase.
114. The method of embodiment 113, wherein the extension reagents include PCR master mix.
115. The method of embodiments 113 or 114, wherein the polymerase includes DNA Polymerase.
116. The method of any of embodiments 86-115, wherein the adjusting the environment includes oscillating the temperature between an annealing temperature, an extension temperature, and a dissociation temperature.
117. The method of embodiment 116, wherein the annealing temperature is 3° C.-5° C. lower than the lowest Tₘ of either the capture sequence template or CSP.
118. The method of embodiments 116 or 117, wherein the annealing temperature is 50° C.-56° C.
119. The method of any of embodiments 116-118, wherein the extension temperature is 70° C.-75° C.
120. The method of any of embodiments 116-119, wherein the extension temperature is 72° C.
121. The method of any of embodiments 116-120, wherein the dissociation temperature is 94° C.-98° C.
122. The method of any of embodiments 86-121, including repeating step (b) until the IC spatial array is produced.
123. The method of any of embodiments 86-122, further including washing out the bridge probes.
124. A method of producing an increased capture potential (IC) spatial array including:
   (a) placing a matrix substrate in contact with a template array within a hybridization buffer, wherein the matrix substrate includes a primer docking element (PDE) anchored to the matrix substrate and the media includes capture strand segments, and wherein the template array includes (1) a plurality of discrete areas, wherein the discrete area contains a plurality of template strands, wherein the template strand includes a PDE template, template strand segments, and a capture sequence template (CST);
   (b) adjusting the environment of the hybridization buffer such that the PDE and capture strand segments hybridize to the template strand;
   (c) adding capture sequence primer (CSP) and DNA ligase to the matrix substrate and template array;
   (d) adjusting the temperature of the hybridization buffer to ligation temperature such that the DNA ligase catalyzes phosphodiester bonds between capture strand segments to form capture probe; wherein the capture probe includes a sequence that hybridizes to the template strand, wherein the capture probe includes a PDE, capture strand segments, and a capture sequence primer (CSP).
125. The method of embodiment 124, wherein the capture strand segments include a first portion of a barcode sequence flanked by nucleotides on at least one side of the first portion of the barcode and a second portion of a barcode sequence flanked by nucleotides on at least one side of the second portion of the barcode.
126. The method of embodiments 124 or 125, wherein the capture strand segments include a first portion of a barcode sequence flanked by at least 6 nucleotides on each side of the first portion of the barcode and a second portion of a barcode sequence flanked by at least 6 nucleotides on each side of the second portion of the barcode.
127. The method of any of embodiments 124-126, wherein the template strand segments include a sequence that hybridizes to the first portion of the barcode sequence flanked by nucleotides on at least one side of the first portion of the barcode and a sequence that hybridizes to the second portion of the barcode sequence flanked by nucleotides on at least one side of the second portion of the barcode.
128. The method of any of embodiments 124-127, wherein the template strand segments include a sequence that hybridizes to the first portion of the barcode sequence flanked by at least 6 nucleotides on each side of the first portion of the barcode and a sequence that hybridizes to the second portion of the barcode sequence flanked by at least 6 nucleotides on each side of the second portion of the barcode.
129. The method of any of embodiments 124-128, wherein the matrix substrate includes a hydrogel.
130. The method of any of embodiments 124-129, wherein the matrix substrate includes a polyacrylamide gel.
131. The method of any of embodiments 124-130, further including isometrically expanding the matrix substrate, wherein isometrically expanding the matrix substrate includes anchoring one or more components of a sample to a gel, followed by gel formation, proteolysis, and swelling.
132. The method of any of embodiments 124-131, further including shrinking the matrix substrate.
133. The method of embodiment 132, wherein shrinking the matrix substrate includes exposing the matrix substrate to a dehydrating solvent, a salt, heat, a vacuum, lyophilization, desiccation, filtration, or air-drying.
134. The method of any of embodiments 124-133, further including isometrically expanding the matrix substrate and shrinking the expanded matrix substrate.
135. The method of any of embodiments 124-134, wherein the matrix substrate is isometrically expanded before producing the IC spatial array and shrunk after producing the IC spatial array.
136. The method of any of embodiments 124-135, wherein the matrix substrate is 50 μm-500 μm thick.
137. The method of any of embodiments 124-136, further including anchoring the PDEs to the matrix substrate with 5′ acrydite.
138. The method of any of embodiments 124-137, wherein the CST includes a polyT sequence.
139. The method of embodiment 138, wherein the polyT sequence includes a 10-50 nucleotide polyT sequence.
140. The method of embodiment 139, wherein the polyT sequence includes

(SEQ ID NO: 2)
TTTTTTTTTTTTTTTTTTTT.

141. The method of any of embodiments 124-137, wherein the CST includes a polyA sequence.
142. The method of embodiment 141, wherein the polyA sequence includes a 10-50 nucleotide polyA sequence.
143. The method of embodiment 142, wherein the polyA sequence includes

(SEQ ID NO: 1)
AAAAAAAAAAAAAAAAAAAA.

144. The method of any of embodiments 124-137, wherein the CST includes a polyU sequence.
145. The method of embodiment 144, wherein the polyU sequence includes a 10-50 nucleotide polyU sequence.
146. The method of embodiment 145, wherein the polyU sequence includes

(SEQ ID NO: 3)
UUUUUUUUUUUUUUUUUUUU.

147. The method of any of embodiments 124-146, wherein the CST includes a recognition sequence.
148. The method of any of embodiments 124-147, wherein the discrete area occupies a discrete x and y location on the template array.
149. The method of any of embodiments 124-148, wherein the barcode template is unique to the position of the template probe on the template array.
150. The method of any of embodiments 124-149, wherein the adjusting the environment of the hybridization buffer includes adjusting the temperature of the hybridization buffer to 80° C. reducing the temperature 0.5° C. per second to an annealing temperature.
151. The method of embodiment 150, wherein the annealing temperature is 45° C. to 50° C.
152. The method of any of embodiments 124-151, wherein the ligation temperature includes room temperature.
153. The method of embodiment 152, wherein room temperature includes 18° C. to 24° C.
154. The method of any of embodiments 124-153, including repeating steps (b) through (d) until the IC spatial array is produced.
155. A method using the increased capture potential (IC) spatial array of any of embodiments 1-34 including:
   (a) situating the IC spatial array with a sample;
   (b) migrating analytes from the sample into the IC spatial array to bind with capture probes, wherein a capture probe includes:
      (1) a barcode that designates the location of the capture probe within the IC spatial array; and
      (2) a capture sequence that binds the analyte;
   (c) generating a product in the IC spatial array, the product including: a sequence or complement thereof of the analyte and a sequence or complement thereof of the barcode; and
   (d) analyzing the product, thereby identifying the location of the analyte within the IC spatial array.
156. The method of embodiment 155, wherein the situating includes contacting the IC spatial array with the sample.
157. The method of embodiment 155, wherein the situating includes contacting the IC spatial array with a depletion layer and contacting the depletion layer with the sample.
158. The method of any of embodiments 155-157, wherein the migrating includes applying an electric field to the sample and IC spatial array such that analytes migrate from the sample to the IC spatial array.
159. The method of embodiments 157 or 158, wherein the depletion layer is separated from the IC spatial array after migrating.
160. The method of any of embodiments 155-159, wherein the sample is permeabilized.
161. The method of any of embodiments 155-160, wherein the generating a product includes performing PCR.
162. The method of any of embodiments 155-160, wherein the generating a product includes isothermal nucleic acid amplification.
163. The method of any of embodiments 155-162, wherein the generating a product further includes amplifying the product.
164. The method of any of embodiments 155-163, wherein the product is cDNA.
165. The method of any of embodiments 155-164, wherein analyzing the product includes sequencing the product.
166. The method of embodiment 165, wherein sequencing the product includes next-generation sequencing, Sanger sequencing, DNA hybridization, or quantitative PCR.
167. The method of embodiment 166, wherein next-generation sequencing includes RNA sequencing.
(x) Example. Two-dimensional (2D) spatial genomic barcodes made of DNA oligonucleotides have been described previously. Such 2D spatial genomic barcodes have millions of spatially defined barcodes that also carry polyT sequences which can be used to capture mRNA (or any other special sequence designed to capture specific oligonucleotide(s)) from a tissue specimen or other biological sample. Usually, each spatial barcode is arrayed/patterned in a 1 μm to 20 μm diameter circle or 1 μm to 20 μm square area. The amount of oligonucleotides that are available to capture mRNA is limited by the surface area in a solid state substrate (glass, silicon, and so on).

Described herein is an improvement that is able to transfer 2D patterned barcodes from a 2D surface to a 3-dimensional (3D) polymeric matrix substrate, such as a hydrogel matrix substrate. The polymeric matrix substrate has more capture sites due to larger surface area provided by the thickness of the 3D matrix substrate. The transfer maintains the distinct and determinable location of each spatial barcode with similar resolution as that provided in a 2D plane.

Optionally, the spatial resolution can be increased (4× to 10×) by using an expanded gel layer during the barcode transfer step.

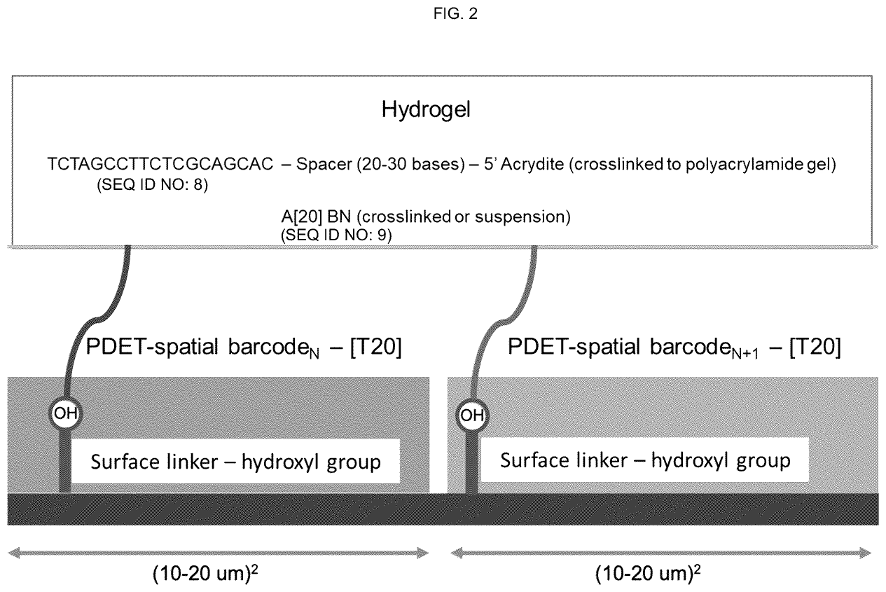

An exemplary transfer mechanism is shown in the FIG. 1A. A hydrogel (50-500 μm thick) with anchored primer docking elements (PDEs) and suspended A [20] oligos is casted or placed (if pre-cast) on top of a 2D spatial barcode array. Both hydrogel and the 2D spatial barcode array are immersed, in a well confined chamber, in standard PCR master mix (such as KAPA Hot Start Master Mix and Taq DNA Polymerase). Standard PCR amplification cycles are applied and the 2D barcodes are automatically transferred to the hydrogel. Since all the spatial barcodes share the same primer docking domains; competition between two adjacent spatial barcodes for amplification component's ensures the spatial resolution is not dispersed during PCR cycles, as shown in FIG. 2.

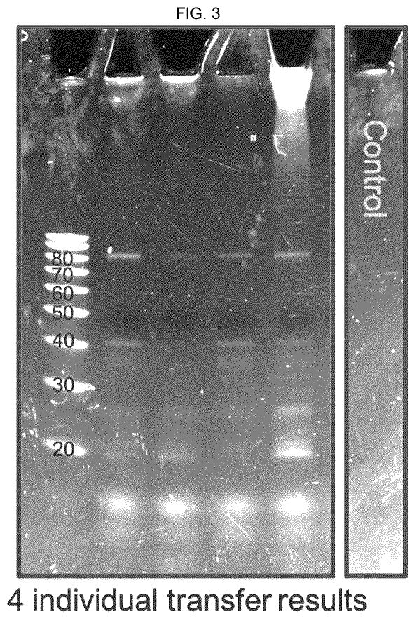

The transfer of a 2D spatial barcode array into a hydrogel matrix substrate has been demonstrated successfully, as illustrated in FIGS. 3-5. After PCR, the hydrogel was heated up to >95° C. to denature the double stranded DNA and elute the complementary strand from the DNAs anchored on the hydrogel matrix substrate. The eluted DNA were run through TB-Urea gel, and showed a correct 80 bases band (FIG. 3). Two oligonucleotides, PDET (complementary to PDE) labeled with Cy3 and A [20] labeled with Alexa-647, were then incubated 1-2 hours with the hydrogel. PDET-Cy3 and A [20]-Alexa-647 diffused in to the gel matrix substrate and hybridized with the anchored DNAs.

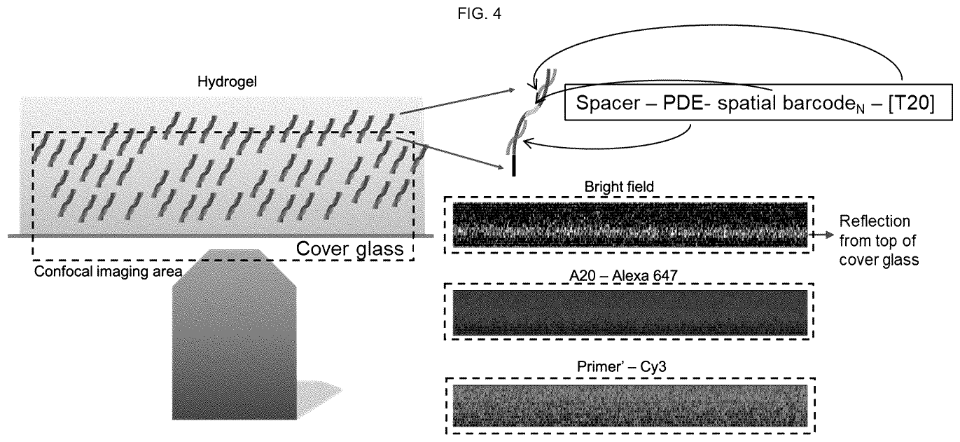

A z-stack confocal image is shown in FIG. 4. The bright field showed a strong reflection signal when the focal plane was set on top surface of the cover glass, both Alexa-647 and Cy3 showed stronger signal after the focal plane passed the reflection plane of the cover glass. This indicates both PDE and T [20] were present in the hydrogel matrix substrate.

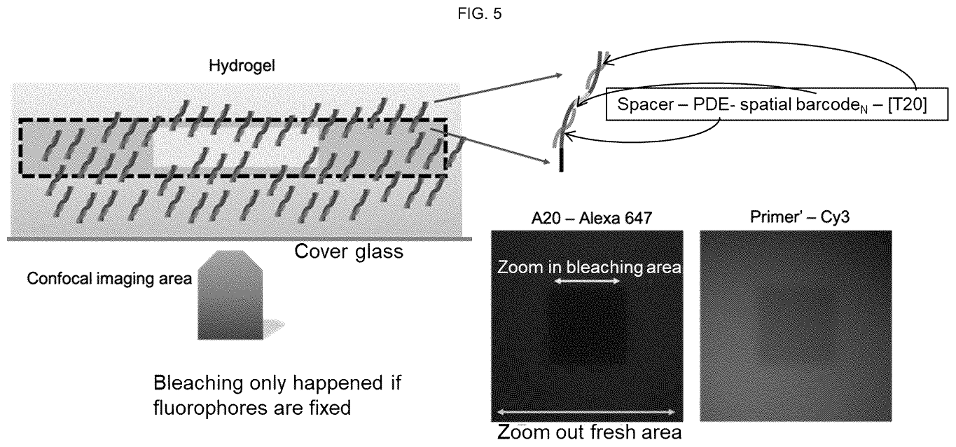

A further confirmation is shown in FIG. 5. When the focal plane was inside the hydrogel, the imaging area was set to a smaller field of view with much stronger intensity of excitation. This bleached the majority of fluorophore in that field of view. A follow-on image was taken after changing to a larger field of view; the clearly visible central dark area indicates anchored and non-suspended fluorophores were presented in the gel area. This bleaching effect will never present if there are only suspended fluorophores

In the FIG. 6A, a tissue section is placed on top of the hydrogel that contains a transferred barcode array within it. The tissue section and the hydrogel are sandwiched by two metal plates and two buffer reservoirs. The buffer reservoirs can be liquid or a soaked polymeric layer that contains high ion concentration for electrophoresis. The metal plate can be copper or platinum. 10-100V voltage bias was applied to the two metal plates for 10-120 seconds to finish the mRNA transfer from tissue section to the gel. All the mRNA driven by electrophoretic force will hybridized to the poly-T (T20) on the 3′ tailing of barcoded DNA. This custom setup can be replaced by a commercial western blot system, such as iBlot or iBlot2.

In addition to reducing lateral diffusion of analytes during transfer, an advantage of combining the provided hydrogel matrix substrate with electrophoretic transfer is the flexibility to add a depletion layer gel in between the tissue sample and the T [20] hydrogel, as shown in the FIG. 6B. Previously described 2D transcriptomic systems were designed to capture all mRNA from the sample, but most RNA signal in such a system will be overwhelmed by house-keeping or less-interesting or highly abundant sequences that limit the capability of studying whole genome expression. In the design shown in FIG. 6B, a hydrogel that is anchored with complementary strands of abundant, less-interesting RNA, such as GAPDH, is placed underneath the sample (between the sample and the spatially barcoded array in the hydrogel matrix substrate). This intervening layer will deplete those abundant genes from the migrating analyte field, and fewer will pass through to be captured by the T [20] oligonucleotides in the spatially barcoded hydrogel matrix substrate. More of T [20] oligonucleotides are then available to capture a larger proportion (or majority) of rarer RNAs among the analytes.
(xi) Closing Paragraphs. The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y., Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridize under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding domain (of, for example, a CAR binding domain or a nanoparticle selected cell targeting ligand) to its cognate binding molecule with an affinity or Kₐ (i.e., an equilibrium association constant of a particular binding interaction with units of 1/M) equal to or greater than 10⁵ M⁻¹, while not significantly associating with any other molecules or components in a relevant environment sample. Binding domains may be classified as “high affinity” or “low affinity”. In particular embodiments, “high affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Kₐ of up to 10⁷ M⁻¹, up to 10⁶ M⁻¹, up to 10⁵ M⁻¹. Alternatively, affinity may be defined as an equilibrium dissociation constant (Kₐ) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Kₐ (equilibrium association constant) for the cognate binding molecule that is higher than the reference binding domain or due to a K_{d} (dissociation constant) for the cognate binding molecule that is less than that of the reference binding domain, or due to an off-rate (K_{off}) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant decrease in analyte capture.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

1. An increased capture potential (IC) spatial array comprising: a matrix substrate, wherein the matrix substrate comprises an array, a thickness, and a plurality of discrete volumes, wherein a discrete volume occupies a distinct x and y position on the array and continues into the thickness of the matrix substrate in a z direction, and wherein the discrete volume comprises a plurality of capture probes comprising a barcode unique to the discrete volume anchored to the matrix substrate and a capture sequence including a 10-50 nucleotide sequence selected from the group of a polyT sequence, polyA sequence, and polyU sequence.

2. The IC spatial array of claim 1, wherein a majority of the plurality of capture probes have a free end.

3.-4. (canceled)

5. The IC spatial array of claim 1, wherein the discrete volume is 1 μm to 30 μm in the x and/or y direction, and/or 1 μm to 3 cm in the z direction.

6-9. (canceled)

10. The IC spatial array of claim 1, wherein the capture sequence binds to a target sequence on an analyte.

11. The IC spatial array of claim 10, wherein the target sequence comprises a sequence selected from the group of a polyA sequence and a polyT sequence, and the analyte is selected from the group of an RNA, a DNA, and a protein.

12-21. (canceled)

22. The IC spatial array of claim 1, wherein the capture probe comprises a functional sequence selected from the group of a cleavage domain, unique molecular identifier, and spacer.

23. (canceled)

24. The IC spatial array of claim 1, wherein the matrix substrate further comprises a composition selected from the group of a hydrogel and a solid state membrane.

25-29. (canceled)

30. The IC spatial array of claim 1, wherein the matrix substrate is 10 μm-2 mm thick.

31. (canceled)

32. The IC spatial array of claim 1, wherein each capture probe of the plurality of capture probes is linked to the matrix substrate with 5′ acrydite.

33.-34. (canceled)

35. A starting matrix substrate comprising primer docking elements (PDEs) and capture sequence primers, wherein the PDEs are anchored to the matrix substrate and the capture sequence primers comprise a 10-50 nucleotide sequence selected from the group of a polyT sequence, polyA sequence, and polyU sequence.

36. The starting matrix substrate of claim 35, wherein the capture sequence primers are or are not anchored to the matrix substrate.

37-50. (canceled)

51. The starting matrix substrate of claim 35, wherein the PDEs are anchored to the matrix substrate with 5′ acrydite.

52.-53. (canceled)

54. The starting matrix substrate of claim 35, wherein the matrix substrate is 50 μm-500 μm thick.

55. The starting matrix substrate of claim 35, wherein the matrix substrate comprises a capillary array.

56-85. (canceled)

86. A method of producing an increased capture potential (IC) spatial array comprising the steps of:
(a) placing a matrix substrate in contact with a template array in a PCR-enabling media, wherein the matrix substrate comprises (1) final primer docking elements (PDEs) anchored to the matrix substrate and (2) capture sequence primers (CSPs), and wherein the template array comprises (1) a plurality of discrete areas, wherein the discrete area contains a plurality of template probes, and wherein the template probes comprise a PDE template, a barcode template, and a capture sequence template;
(b) adjusting the environment of the PCR-enabling media such that:
(i) the CSPs hybridize with the capture sequence templates such that the CSPs hybridized to the capture sequence templates extend through the template probes to create bridge probes, wherein the bridge probe comprises a sequence that hybridizes to the template probe and comprises the CSP, a bridge barcode, and a bridge PDE;
(ii) the bridge probes dissociate from the template probes;
(iii) the bridge PDEs of the bridge probes hybridize with the PDEs of the matrix substrate; such that the PDEs of the matrix substrate hybridized to the bridge PDEs extend through the bridge probes to create capture probes, wherein the capture probe comprises a sequence that hybridizes to the bridge probe and comprises a capture sequence, a barcode, and the PDE of the matrix substrate; and
(iv) the capture probes dissociate from the bridge probes, wherein the capture sequence and capture sequence template have the same sequence and are complements to the CSP, the barcode and barcode template have the same sequence and are complements to the bridge barcode, and the PDE of the matrix substrate and PDE template are the same sequence and are complements to the bridge PDE.

87.-88. (canceled)

89. The method of claim 86, further comprising the step of isometrically expanding the matrix substrate, wherein isometrically expanding the matrix substrate comprises anchoring one or more components of a sample to a gel, followed by gel formation, proteolysis, and swelling.

90. (canceled)

91. The method of claim 86, further comprising the step of shrinking the matrix substrate, wherein shrinking the matrix substrate comprises exposing the matrix substrate to a dehydrating solvent, a salt, heat, a vacuum, lyophilization, desiccation, filtration, or air-drying.

92. (canceled)

93. The method of claim 86, wherein the matrix substrate is isometrically expanded before producing the IC spatial array and then shrunk after producing the IC spatial array.

94-98. (canceled)

99. The method of claim 86, wherein the capture sequence template comprises a 10-50 nucleotide sequence selected from the group of a polyT sequence, polyA sequence, and polyU sequence.

100-115. (canceled)

116. The method of claim 86, wherein the adjusting the environment comprises oscillating a temperature of the PCR-enabling media between an annealing temperature, an extension temperature, and a dissociation temperature, wherein the annealing temperature is 50° C.-56° C., the extension temperature is 70° C.-75° C., and the dissociation temperature is 94° C.-98° C.

117-154. (canceled)

155. A method of using the increased capture potential (IC) spatial array of claim 1 comprising the steps of:
(a) situating the IC spatial array with a sample;
(b) migrating analytes from the sample into the IC spatial array to bind with capture probes, wherein a capture probe comprises:
(1) a barcode that designates the location of the capture probe within the IC spatial array; and
(2) a capture sequence that binds the analyte;
(c) generating a product in the IC spatial array, the product comprising: a sequence or complement thereof of the analyte and a sequence or complement thereof of the barcode; and
(d) analyzing the product, thereby identifying the location of the analyte within the IC spatial array.

156-167. (canceled)


SEQUENCE LISTING (14 sequences)

SEQ ID NO: 1 - DNA, 20 bp, synthetic construct
  source 1..20: mol_type = other DNA
aaaaaaaaaaaaaaaaaaaa

SEQ ID NO: 2 - DNA, 20 bp, synthetic construct
  source 1..20: mol_type = other DNA
tttttttttttttttttttt

SEQ ID NO: 3 - RNA, 20 nt, synthetic construct
  source 1..20: mol_type = other RNA
tttttttttttttttttttt

SEQ ID NO: 4 - DNA, 16 bp, synthetic construct
  source 1..16: mol_type = other DNA
tcccgtacagaagacg

SEQ ID NO: 5 - DNA, 19 bp, synthetic construct
  source 1..19: mol_type = other DNA
cacgacgctcttccgatct

SEQ ID NO: 6 - DNA, 15 bp, synthetic construct
  source 1..15: mol_type = other DNA
gggcatgtcttctgc

SEQ ID NO: 7 - DNA, 19 bp, synthetic construct
  source 1..19: mol_type = other DNA
tctagccttctcgcagcac

SEQ ID NO: 8 - DNA, 19 bp, synthetic construct
  source 1..19: mol_type = other DNA
tctagccttctcgcagcac

SEQ ID NO: 9 - DNA, 22 bp, synthetic construct
  source 1..22: mol_type = other DNA
aaaaaaaaaaaaaaaaaaaabn

SEQ ID NO: 10 - DNA, 71 bp, synthetic construct
  source 1..71: mol_type = other DNA
cacgacgctcttccgatctcannnnnnnactctcacacaannnnnnncacatttttttttttttttttttt

SEQ ID NO: 11 - DNA, 15 bp, synthetic construct
  source 1..15: mol_type = other DNA
  misc_feature 15: note = phosphate group
gtgctgcgagaaggc

SEQ ID NO: 12 - DNA, 19 bp, synthetic construct
  source 1..19: mol_type = other DNA
  misc_feature 19: note = phosphate group
tagagtnnnnnnntgagag

SEQ ID NO: 13 - DNA, 19 bp, synthetic construct
  source 1..19: mol_type = other DNA
  misc_feature 19: note = phosphate group
tgtgttnnnnnnngtgtaa

SEQ ID NO: 14 - DNA, 18 bp, synthetic construct
  source 1..18: mol_type = other DNA
aaaaaaaaaaaaaaaaaa